(19) United States
(12) Patent Application Publication
(10) Pub. No.: US 2022/0106616 A1
(43) Pub. Date: Apr. 7, 2022

(54) PRODUCTION OF CANNABINOIDS IN FILAMENTOUS FUNGI

(71) Applicant: DYADIC INTERNATIONAL (USA), INC., Jupiter, FL (US)

(72) Inventors: Mark Aaron EMALFARB, Jupiter, FL (US); Ronen TCHELET, Budapest (HU); Gabor KERESZTES, Pilisborosjeno (HU); Marja Hannele ILMEN, Helsinki (FI); Outi Mirjami KOIVISTOINEN, Espoo (FI); Kari Tapio KOIVURANTA, Vantaa (FI); Paula JOUHTEN, Espoo (FI); Sandra CASTILLO, Espoo (FI)

(21) Appl. No.: 17/429,461

(22) PCT Filed: Feb. 10, 2020
(86) PCT No.: PCT/IB2020/051015

§ 371 Date: Aug. 9, 2021

Related U.S. Application Data
(60) Provisional application No. 62/803,498, filed on Feb. 10, 2019.
(60) Provisional application No. 62/906,133, filed on Sep. 26, 2019.

(51) Int. Cl. C12P 7/42; C12N 15/113

(52) U.S. Cl. CPC C12P 7/42; C12R 2001/645; C12N 15/113

(57) ABSTRACT

The present invention relates to genetically modified ascomycetous filamentous fungi, particularly of the species Thermothelomyces heterothallica capable of producing cannabinoids and precursors thereof, particularly of producing cannabigerolic acid (CBGA) and/or cannabigerovarinic acid (CBGVA) and products thereof, including tetrahydrocannabinolic acid (THCA), cannabidiolic acid (CBDA) and cannabidivarinic acid (CBDVA), and use thereof for producing said precursors and cannabinoids.

### FIELD OF THE INVENTION

The present invention relates to genetically modified ascomycetous filamentous fungi, particularly of the species Thermothelomyces heterothallica (formerly Mycehophthora thermophila) capable of producing cannabinoids and precursors thereof, particularly of producing cannabigerolic acid (CBGA) and/or cannabigerovarinic acid (CBGVA) and products thereof, including tetrahydrocannabinolic acid (THCA), cannabidiolic acid (CBDA), and cannabidivarinic acid (CBDVA), and uses thereof for producing said precursors and cannabinoids.

### BACKGROUND OF THE INVENTION

Plants from the genus Cannabis have been used by humans for their medicinal properties for thousands of years. The bioactive effects of Cannabis have been attributed to a class of compounds termed “cannabinoids,” of which there are hundreds of structural analogs including tetrahydrocannabinol (THC) and cannabidiol (CBD). Cannabinoid physiological effects are attributed to their interaction with cannabinoid receptors and other target molecules found in humans and other animals. Cannabinoid receptor type 1 (CB1) is common in the brain, the reproductive system, and the eye. Cannabinoid receptor type 2 (CB2) is common in the immune system and mediates therapeutic effects related to inflammation in animal models.

Cannabinoids and preparations of Cannabis material have recently found application as therapeutics for chronic pain, multiple sclerosis, cancer-associated nausea and vomiting, weight loss, appetite loss, spasticity, and other conditions.

Cannabinoids for pharmaceutical or nutraceutical use are currently produced by chemical synthesis or through the extraction of cannabinoids from plants that are producing these cannabinoids, typically from Cannabis sativa (C. sativa).

Use of plant-derived cannabinoids encounters several obstacles. Different cannabinoid profile will have different pharmaceutical effects. However, the amounts and profile of the cannabinoids produced by plants are variable, even within plants of single variety; the extraction method used further affect the cannabinoids profile within the extracted composition; and the cannabinoid profile includes compounds that do not have any therapeutic effects. Taken together, the crude nature of plant-derived cannabinoid extracts is an obstacle in their use as pharmaceutical drugs.

While synthetic cannabinoid compounds have been approved by the FDA, the chemical synthesis is a costly process, involves the use of chemicals that are not environmentally friendly, and, most importantly, various chemically synthesized cannabinoids have been classified as less pharmacologically active as those extracted from plants (particularly from C. sativa).

Attempts have been made to develop strategies for producing cannabinoids in microorganisms. For example, U.S. Pat. Nos. 9,611,460 and 10,059,971 disclose nucleic acid molecules encoding polypeptides having polyketide synthase activity. Expression or over-expression of the nucleic acids alters levels of cannabinoid compounds in organisms, particularly yeast and bacteria. The polypeptides may be used in vivo or in vitro to produce cannabinoid compounds.

U.S. Pat. Nos. 9,822,384 and 10,093,949 further disclose genetically engineered microorganisms, such as yeast or bacteria, to produce cannabinoids by inserting genes that produce the appropriate enzymes for the metabolic production of a desired compound.

International Application Publication No. WO 2011/017798 discloses nucleic acid molecules isolated from C. sativa encoding polypeptides having aromatic prenyltransferase activity. Specifically, the enzyme, C. sativa CBGAS PT1, is a geranyl pyrophosphate olivetolate geranyltransferase, active in the cannabinoid biosynthesis step of prenylation of olivetolic acid to form cannabigerolic acid (CBGA). Expression or over-expression of the nucleic acids alters levels of cannabinoid compounds.

International Application Publication No. WO/2017/139496 discloses genetically engineered microorganisms comprising one or more genetic modifications that increase expression of a Type I Fatty Acid Synthase alpha (FASa) and a Fatty Acid Synthase beta (FASP) relative to a microorganism of the same species without the one or more genetic modifications, wherein the genetically modified microorganism has increased production of hexanoic acid relative to an unmodified organism of the same species.

International Application Publication No. WO 2018/200888 discloses genetically modified host cells, that produce a cannabinoid, a cannabinoid derivative, a cannabinoid precursor, or a cannabinoid precursor derivative and methods of synthesizing same. Particularly, the genetically modified host cell comprises one or more heterologous nucleic acids encoding a geranyl pyrophosphate:olivetolic acid geranyltransferase (GOT) polypeptide, which catalyzes the production of cannabigerolic acid from geranyl pyrophosphate (GPP) and olivetolic acid in an amount higher than the amount produced by hitherto known enzyme. Fungal cells are proposed, inter alia, as host cells.

Wild type Thermothelomyces heterothallica (Th. heterothallica) C1 (recently renamed from Myceliophthora thermophila, which in term was renamed from Chrysosporium lucknowense) is a thermos-tolerant ascomycetous filamentous fungus producing high levels of cellulases, which made it attractive for production of these and other enzymes on a commercial scale.

For example, U.S. Pat. Nos. 8,268,585 and 8,871,493 to the Applicant of the present invention disclose a transformation system in the field of filamentous fungal hosts for expressing and secreting heterologous proteins or polypeptides. Also disclosed is a process for producing large amounts of polypeptide or protein in an economical manner. The system comprises a transformed or transfected fungal strain of the genus Chrysosporium, more particularly of Chrysosporium lucknowense and mutants or derivatives thereof. Also disclosed are transformants containing Chrysosporium coding sequences, as well expression-regulating sequences of Chrysosporium genes.

Wild type C1 was deposited in accordance with the Budapest Treaty with the number VKM F-3500 D, deposit date Aug. 29, 1996. High Cellulase (HC) and Low Cellulase (LC) strains have also been deposited, as described, for example, in U.S. Pat. No. 8,268,585.

There remains a need for a system for producing high amounts of pure cannabinoids for use in the pharmaceutical industry in an efficient and cost-effective way.

### SUMMARY OF THE INVENTION

The present invention provides genetically modified ascomycetous filamentous fungi, capable of producing cannabinoids, cannabinoid precursors and derivatives thereof. Particularly, the present invention provides Thermothelomyces heterothallica strain C1 as an exemplary ascomycetous filamentous fungus genetically modified to enable the production of cannabigerolic acid (CBGA) and/or cannabigerovarinic acid (CBGVA) and products thereof, including cannabidiolic acid (CDBA), Δ9-tetrahydrocannabinolic acid (THCA) and cannabidivarinic acid (CBDVA).

According to certain aspects the present invention provides production of cannabinoids, cannabinoid precursors and derivatives thereof by means of production by fermentation, where the said compounds are produced in vivo in the transgenic fungus during fermentation, and/or by production by bioconversion, where said compounds are produced from precursors in vitro using cell lysates, cell extracts or purified enzymes as biocatalysts produced by fermentation, particularly of/from the transgenic fungi of the invention, and/or by any combination of thereof, where a precursor is produced in vivo during fermentation, and that precursor is further modified in vitro using cell lysates, cell extracts or purified enzymes either produced by fermentation or otherwise, as a catalyst. The cannabinoids or cannabinoid precursors produced by the genetically modified fungi of the invention may form final products to be used, or may be amenable to further in vitro modifications to produce further products. For example, CBGA produced by the fungi of the invention can be used for in vitro production of any one of THC, CBD and derivatives thereof.

The yeast Saccharomyces cerevisiae (S. cerevisiae) is currently the major candidate for the production of cannabinoids in microorganisms. Surprisingly, the present invention shows that Th. heterothallica, exemplifying ascomycetous filamentous fungi, is capable of harnessing endogenous pathways naturally producing cannabinoid precursor molecules, for down-stream pathway steps catalyzed by the exogenous enzymes expressed in the transgenic fungi of the invention. Th. heterothallica C1 and other filamentous fungi encode in their genomes for example phosphoketolases that enhance the production of cytosolic acetyl-CoA. These phosphoketolases are not present in S. cerevisiae. Acetyl-CoA is a precursor for both Hexanoyl-CoA and Geranyl Pyrophosphate (GPP), which are the two essential precursor molecules in the pathway of cannabinoid production. Without wishing to be bound by any specific theory or mechanism of action, harnessing the endogenous fatty acids biosynthesis of fungi, and optionally optimizing specific steps in the pathway leading to the production of cannabinoid precursor, contribute to the advantage of filament fungi as a “factory” for cannabinoids.

The exemplary Th. heterothallica C1 system of the present invention shows high biomass production, and can secrete cellular-produced proteins and secondary metabolite at higher rate compared to yeast strains and also compared to other ascomycetous filamentous fungal strains when grown under suitable conditions. Without wishing to be bound by any specific theory or mechanism of action, diverting the resources of the fungus from the production of secreted proteins and/or biomass by methods of metabolic engineering to secondary metabolites further increases the potential of this strain to become a more efficient host compared to for example, S. cerevisiae.

Furthermore, several Th. heterothallica C1 strains developed by the Applicant of the present invention are less sensitive to feedback repression by glucose and other fermentable sugars present in the growth medium as carbon source than conventional yeast strains and also most other ascomycetous filamentous fungal hosts, and consequently can tolerate higher feeding rate of the carbon source, leading to high yields production by this fungus.

In addition, some of the Th. heterothallica C1 strains developed by the Applicant of the present invention can be grown in liquid cultures with significantly reduced medium viscosity in fermenters, compared to most other ascomycetous filamentous fungal species. The low viscosity cultures of Th. heterothallica C1 are comparable to that of S. cerevisiae and other yeast species. The low viscosity may be attributed to the morphological change of the strain from having long and highly interlaced hyphae in the parental strain(s) to short and less interlaced hyphae in the developed strain(s). Low medium viscosity is highly advantageous in large scale industrial production. For example, Th. heterothallica C1 strain UV18-25, deposit No. VKM F-3631 D, and its derivatives, which show reduced sensitivity to glucose repression, has been grown industrially to produce recombinant enzymes at volumes of more than 100,000 liters.

According to a first aspect, the present invention provides a genetically modified ascomycetous filamentous fungus for producing at least one cannabigerolic acid, at least one cannabigerolic acid precursor molecule and/or at least one cannabigerolic acid product, and derivatives thereof, wherein the genetically modified filamentous fungus comprises at least one cell comprising at least one of (i) at least one heterologous polynucleotide encoding olivetol synthase (OLS); (ii) at least one heterologous polynucleotide encoding olivetolic acid cyclase (OAC); (iii) at least one heterologous polynucleotide encoding prenyltransferase (PT) having cannabigerolic acid synthase (CBGAS) activity; (iv) at least one heterologous polynucleotide encoding cannabidiolic acid synthase (CBDAS); and (v) at least one heterologous polynucleotide encoding tetrahydrocannabinolic acid synthase (THCAS).

According to certain embodiments, the genetically modified ascomycetous filamentous fungus comprises at least one cell comprising (i) at least one heterologous polynucleotide encoding olivetol synthase (OLS) and (ii) at least one heterologous polynucleotide encoding olivetolic acid cyclase (OAC). According to certain exemplary embodiments, this genetically modified ascomycetous filamentous fungus is capable of producing olivetolic acid and/or divarinolic acid.

According to certain embodiments, the genetically modified ascomycetous filamentous fungus comprises at least one cell comprising (i) at least one heterologous polynucleotide encoding olivetol synthase (OLS); (ii) at least one heterologous polynucleotide encoding olivetolic acid cyclase (OAC); and (iii) at least one heterologous polynucleotide encoding prenyltransferase (PT) having cannabigerolic acid synthase (CBGAS) activity. According to certain exemplary embodiments, this genetically modified ascomycetous filamentous fungus is capable of producing cannabigerolic acid (CBGA) and/or cannabigerovarinic acid (CBGVA).

According to certain embodiments, the genetically modified ascomycetous filamentous fungus comprises at least one cell comprising (i) at least one heterologous polynucleotide encoding olivetol synthase (OLS); (ii) at least one heterologous polynucleotide encoding olivetolic acid cyclase (OAC); (iii) at least one heterologous polynucleotide encoding prenyltransferase (PT) having cannabigerolic acid synthase (CBGAS) activity; and (iv) at least one heterologous polynucleotide encoding cannabidiolic acid synthase (CBDAS). According to certain exemplary embodiments, this genetically modified ascomycetous filamentous fungus is capable of producing cannabidiolic acid (CBDA) and/or cannabigerovarinic acid (CBGVA).

According to certain embodiments, the genetically modified ascomycetous filamentous fungus comprises at least one cell comprising (i) at least one heterologous polynucleotide encoding olivetol synthase (OLS); (ii) at least one heterologous polynucleotide encoding olivetolic acid cyclase (OAC); (iii) at least one heterologous polynucleotide encoding prenyltransferase (PT) having cannabigerolic acid synthase (CBGAS) activity; and (iv) at least one heterologous polynucleotide encoding tetrahydrocannabinolic acid synthase (THCAS). According to certain exemplary embodiments, this genetically modified ascomycetous filamentous fungus is capable of producing tetrahydrocannabinolic acid (THCA).

According certain embodiments, the genetically modified fungus capable of producing cannabinoids and their precursors of the present invention is further modified to produce elevated amount of the cannabigerolic acid precursor molecule hexanoyl-CoA. According to certain embodiments, the fungus is modified to produce elevated amount of hexanoyl-CoA by modifying the endogenous fatty acid synthesis pathway. According to certain embodiments, the fungus is modified to produce elevated amount of hexanoyl-CoA by further transforming the at least one cell with at least one exogenous polynucleotide encoding hexanoate synthase, at least one exogenous polynucleotide encoding acyl-activating enzyme (AAE) or a combination thereof.

According certain embodiments, the genetically modified fungus capable of producing cannabinoids and their precursors of the present invention is further modified to produce elevated amount of the cannabigerolic acid precursor molecule Geranyl Pyrophosphate (GPP). According to certain embodiments, the fungus is modified to produce elevated amount of GPP by modifying the fungus endogenous GPP synthesis pathway. According to certain embodiments, the fungus is modified to produce elevated amount of GPP by further transforming the at least one cell with at least one endogenous or heterologous polynucleotide encoding GPP-synthetase enzyme (GPPS) and/or a 3-Hydroxy 3-methylglutaryl-CoA (HMG-CoA) reductase enzyme (HMGCR).

According certain embodiments, the genetically modified fungus capable of producing increased amounts of cannabinoids and their precursors of the present invention is further modified to produce elevated amount of the cannabigerolic acid precursor molecules hexanoyl-CoA and Geranyl Pyrophosphate (GPP) by means as described hereinabove.

According certain embodiments, the genetically modified fungus capable of producing increased amounts of cannabinoids and their precursors of the present invention is even further modified to produce elevated amount of cytoplasmic Acetyl-CoA levels. According to certain embodiments, the fungus is modified to produce elevated amount of cytoplasmic Acetyl-CoA by modifying the endogenous fatty acid synthesis pathway. According to certain embodiments, the fungus is modified to produce elevated amount of cytoplasmic Acetyl-CoA by further transforming the at least one cell with at least one endogenous or heterologous polynucleotide encoding phosphoketolase and/or acetylphosphatase.

According to certain embodiments, the various strains generated as described above are capable producing enzyme activities that as cell extracts, enzyme extracts or purified enzymes enable the production of cannabinoids and their derivatives in vitro.

According to certain embodiments, the OLS comprises an amino acid sequence at least 75% homologous to the amino acid sequence of C. sativa OLS. According to certain exemplary embodiments, the C. sativa OLS comprises the amino acid sequence set forth in SEQ ID NO:1.

According to certain embodiments, the OAC comprises an amino acid sequence at least 75% homologous to the amino acid sequence of C. sativa OAC. According to certain exemplary embodiments, the C. sativa OAC comprises the amino acid sequence set forth in SEQ ID NO:3.

According to certain embodiments, the prenyltransferase (PT) having CBGAS activity comprises an amino acid sequence at least 75% homologous to the amino acid sequence of any one of C. sativa PT4, C. sativa PT1 and Streptomyces sp. 190 NphB protein. According to certain exemplary embodiments, the C. sativa PT1 comprises the amino acid sequence set forth in SEQ ID NO:5. According to certain exemplary embodiments, the C. sativa PT4 comprises the amino acid sequence set forth in SEQ ID NO:7 or a part thereof. According to certain exemplary embodiments, the Streptomyces sp. 190 NphB comprises the amino acid sequence set forth in SEQ ID NO:9. According to certain currently exemplary embodiments, the prenyltransferase (PT) having CBGAS activity used according to the teachings of the present invention is C. sativa PT4 having the amino acid sequence set forth in SEQ ID NO:7 or a part thereof. According to certain additional or alternative embodiments, the PT4 is a mature protein lacking a signal peptide (PT4t) comprising the nucleic acid sequence set forth in SEQ ID NO:89.

According to certain embodiments, the CBDAS comprises an amino acid sequence at least 75% homologous to the amino acid sequence of C. sativa CBDAS. According to certain exemplary embodiments, the C. sativa CBDAS comprises the amino acid sequence set forth in SEQ ID NO:11 or a part thereof. According to certain embodiments, the C. sativa CBDAS is a mature protein lacking a signal peptide, said mature protein comprises the amino acid sequence set forth in SEQ ID NO:90.

According to certain embodiments, the THCAS comprises an amino acid sequence at least 75% homologous to the amino acid sequence of C. sativa THCAS. According to certain exemplary embodiments, the C. sativa THCAS comprises the amino acid sequence set forth in SEQ ID NO:13 or a part thereof. According to certain embodiments, the C. sativa THCAS is a mature protein lacking a signal peptide comprising amino acids 2-28 of SEQ ID NO:13.

According to certain embodiments, the filamentous fungus genus is selected from the group consisting of Thermothelomyces, Myceliophthora, Aspergillus, Penicillium, Trichoderma, Rasamsonia, Chrysosporium, Corynascus, Fusarium, Neurospora, Talaromyces and the like.

According to certain exemplary embodiments, the filamentous fungus is selected from the group consisting of Thermothelomyces thermophila (formerly M. thermophila), Thermothelomyces heterothallica (formerly M. thermophila and heterothallica), Myceliophthora lutea, Aspergillus nidulans, Penicillium chrysogenum, Trichoderma reesei, and Rasamsonia emersonii.

According to certain currently exemplary embodiments, the polynucleotides of the present invention are designed based on the amino acid sequence of the enzyme to be produced employing a codon usage of a filamentous fungus.

According to certain exemplary embodiments, the fungus is Th. heterothallica and the polynucleotide encoding the enzyme cascade of the invention are optimized for expression in this fungus. According to these embodiments, the polynucleotide encoding OLS comprises the nucleic acid sequence set forth in SEQ ID NO:2; the polynucleotide encoding OAC comprises the nucleic acid sequence set forth in SEQ ID NO:4; the polynucleotide encoding C. sativa PT1 comprises the nucleic acid sequence set forth in SEQ ID NO:6; the polynucleotide encoding C. sativa PT4 comprises the nucleic acid sequence set forth in SEQ ID NO:8 and the polynucleotide encoding mature C. sativa PT4 without signal peptide comprises the nucleic acid sequence set forth in SEQ ID NO:88; the polynucleotide encoding Streptomyces sp. 190 NphB protein comprises the nucleic acid sequence set forth in SEQ ID NO:10; and the polynucleotide encoding C. sativa CBDAS comprises the nucleic acid sequence set forth in SEQ ID NO:12 and the polynucleotide encoding the mature protein without signal peptide comprises the nucleic acid sequence set forth in SEQ ID NO:91.

The polynucleotides encoding each of the enzymes may form part of one or more DNA constructs and/or expression vectors. According to certain embodiments, each of the polynucleotide forms part of a separate expression DNA construct/vector. According to other embodiments, part or all the polynucleotides are present within the same DNA construct/expression vector.

According to certain embodiments, culturing of the genetically modified fungus in a suitable medium provides for synthesis of the cannabigerolic acid, cannabigerolic acid precursor and/or cannabigerolic acid product, and/or derivatives thereof in an increased amount compared to the amount produced in a corresponding unmodified fungus cultured under similar conditions.

According to certain embodiments, the corresponding unmodified fungus is of the same species of the genetically modified fungus. According to some embodiments, the corresponding fungus is isogenic to the genetically modified fungus.

According to certain embodiments, the cannabigerolic acid precursor is selected from the group consisting of hexanoic acid, olivetolic acid, GPP, derivatives thereof and any combination thereof. Each possibility represents a separate embodiment of the present invention.

Cannabigerolic acid is the precursor of a large number of cannabinoids. The genetically modified fungi of the present invention can thus be used for the production of all such cannabinoids and derivatives thereof.

According to certain exemplary embodiments, the present invention provides a genetically modified ascomycetous filamentous fungus producing cannabigerolic acid and derivatives thereof. According to certain embodiments, the genetically modified ascomycetous filamentous fungus is Th. heterothallica. According to certain currently exemplary embodiments, the genetically modified ascomycetous filamentous fungus is Th. heterothallica C1. According to these embodiments, the genetically modified C1 fungus comprises at least one cell comprising (i) at least one heterologous polynucleotide encoding olivetol synthase (OLS); (ii) at least one heterologous polynucleotide encoding olivetolic acid cyclase (OAC); and (iii) at least one heterologous polynucleotide encoding prenyltransferase (PT) having cannabigerolic acid synthase (CBGAS) activity.

According to certain additional or alternative exemplary embodiments, the present invention provides a genetically modified ascomycetous filamentous fungus producing cannabidiolic acid and/or derivatives thereof, cannabidiolic acid products and/or derivatives thereof, and any combination thereof. According to certain embodiments, the genetically modified ascomycetous filamentous fungus is Th. heterothallica. According to certain currently exemplary embodiments, the genetically modified ascomycetous filamentous fungus is Th. heterothallica C1. According to these embodiments, the genetically modified C1 fungus comprises at least one cell comprising (i) at least one heterologous polynucleotide encoding olivetol synthase (OLS); (ii) at least one heterologous polynucleotide encoding olivetolic acid cyclase (OAC); (iii) at least one heterologous polynucleotide encoding prenyltransferase (PT) having cannabigerolic acid synthase (CBGAS) activity; and (iv) at least one heterologous polynucleotide encoding cannabidiolic acid synthase (CBDAS).

According to certain additional or alternative exemplary embodiments, the present invention provides a genetically modified ascomycetous filamentous fungus producing tetrahydrocannabinolic acid. According to certain embodiments, the genetically modified ascomycetous filamentous fungus is Th. heterothallica. According to certain currently exemplary embodiments, the genetically modified ascomycetous filamentous fungus is Th. heterothallica C1. According to these embodiments, the genetically modified C1 fungus comprises at least one cell comprising (i) at least one heterologous polynucleotide encoding olivetol synthase (OLS); (ii) at least one heterologous polynucleotide encoding olivetolic acid cyclase (OAC); (iii) at least one heterologous polynucleotide encoding prenyltransferase (PT) having cannabigerolic acid synthase (CBGAS) activity; and (iv) at least one heterologous polynucleotide encoding tetrahydrocannabinolic acid synthase (THCAS).

It is to be understood explicitly that the scope of the present invention encompasses homologs, analogs, variants and derivatives, including shorter and longer polypeptides, proteins and polynucleotides, as well as polypeptide, protein and polynucleotide analogs with one or more amino acid or nucleic acid substitution, as well as amino acid or nucleic acid derivatives, non-natural amino or nucleic acids and synthetic amino or nucleic acids as are known in the art, with the stipulation that these variants and modifications must preserve the activity of enzymes described herein. Specifically, any active fragments of the active polypeptide or protein as well as extensions, conjugates and mixtures are disclosed according to the principles of the present invention.

It is to be understood that any combination of each of the aspects and the embodiments disclosed herein is explicitly encompassed within the disclosure of the present invention.

Other objects, features and advantages of the present invention will become clear from the following description and drawings.

### BRIEF DESCRIPTION OF THE FIGURES

FIG. 1 demonstrates production of olivetolic acid by Th. heterothallica transformed with OLS and OAC encoding polynucleotides.

FIG. 2 shows that synthesis of olivetolic acid may be increased by further transforming the fungus with AAE1 encoding polynucleotides as in strain S3594.

FIG. 3 demonstrates that expression of AAE1 results in higher OA synthesis compared to expression of AAE3, when each of the enzyme is expressed together with OLS and OAC (Strain M3275 and S3277, respectively).

FIG. 4 shows that strains comprising mature PT4 peptide without the signal peptide (PT4t) are suitable for the production of CBGA.

### DETAILED DESCRIPTION OF THE INVENTION

The present invention provides alternative, highly efficient system for producing pure cannabinoid products, particularly cannabidiolic acid (CBDA) and cannabidiol (CBD) as well as tetrahydrocannabinolic acid (THCA), tetrahydrocannabinol (THC) and derivatives thereof. The system of the invention is based in part on the filamentous fungus Thermothelomyces heterothallica C1 and particular strains thereof, which have been previously developed as a natural biological factory for protein production. These strains show high growth rate while keeping low culture viscosity, and are thus highly suitable for continuous growth in fermentation cultures at volumes as high as 100,000-150,000 liters or greater.

### Definitions

Ascomycetous filamentous fungi as defined herein refer to any fungal strain belonging to the group Pezizomycotina. The Pezizomycotina comprises, but is not limited to the following groups:

Sordariales, including genera:
Thermothelomyces (including species: heterothallica and thermophila),
Myceliophthora (including the species lutea and unnamed species),
Corynascus (including the species fumimontanus),
Neurospora (including the species crassa);

Hypocreales, including genera:
Fusarium (including the species graminearum and venenatum),
Trichoderma (including the species reesei, harzianum, longibrachiatum and viride);

Onygenales, including genera:
Chrysosporium (including the species lucknowense);

Eurotiales, including genera:
Rasamsonia (including the species emersonii),
Penicillium (including the species verrucosum),
Aspergillus (including the species funiculosus, nidulans, niger and oryzae)
Talaromyces (including the species piniphilus (formerly Penicillium funiculosum);

It is to be understood that the above list is not conclusive, and is meant to provide an incomplete list of industrially relevant filamentous ascomycetous fungal species.

While there may be filamentous ascomycetous species outside Pezizomycotina, that group does not contain Saccharomycotina, which contains most commonly known non-filamentous industrially relevant genera, such as Saccharomyces, Komagataella (including formerly Pichia pastoris), Kluyveromyces or Taphrinomycotina, which contains some other commonly known non-filamentous industrially relevant genera, such as Schizosaccharomyces.

All taxonomical categories above are defined according to the NCBI Taxonomy browser (ncbi.nlm.nih.gov/taxonomy) as of the date of the patent application.

It must be appreciated that fungal taxonomy is in constant move, and the naming and the hierarchical position of taxa may change in the future. However, a skilled person in the art will be able to unambiguously determine if a particular fungal strain belongs to the group as defined above.

According to certain embodiments, the filamentous fungus genus is selected from the group consisting of Thermothelomyces, Myceliophthora, Aspergillus, Penicillium, Trichoderma, Rasamsonia, Chrysosporium, Corynascus, Fusarium, Neurospora, Talaromyces and the like. According to some embodiments, the fungus is selected from the group consisting of Thermothelomyces thermophila (formerly M. thermophila), Thermothelomyces heterothallica (formerly M. thermophila and heterothallica), Myceliophthora lutea, Aspergillus nidulans, Aspergillus funiculosus Aspergillus niger, Aspergillus oryzae, Penicillium chrysogenum, Penicillium verrucosum, Trichoderma reesei, Trichoderma harzianum, Trichoderma longibrachiatum, Trichoderma viride, Chrysosporium lucknowense, Rasamsonia emersonii, Sporotrichum thermophile, Corynascus fumimontanus, Corynascus thermophilus, Fusarium graminearum, Fusarium venenatum, Neurospora crassa, and Talaromyces piniphilus.

Particularly, the present invention provides Th. heterothallica strain C1 as model for an ascomycetous filamentous fungus, capable of producing cannabinoids, cannabinoid precursors and derivatives thereof.

The terms “Thermothelomyces” and its species “Thermothelomyces heterothallica and thermophila” are used herein in the broadest scope as is known in the art. Description of the genus and its species can be found, for example, in Marin-Felix Y (2015. Mycologica 107(3): 619-632 doi.org/10.3852/14-228) and van den Brink J et al. (2012, Fungal Diversity 52(1):197-207). As used herein “C1” or “Thermothelomyces heterothallica C1” or Th. heterothallica C1, or C1 all refer to Thermothelomyces heterothallica strain C1.

It is noted that the above authors (Marin-Felix et al., 2015) proposed splitting of the genus Myceliophthora based on differences in optimal growth temperature, morphology of the conidiospore, and details of the sexual reproduction cycle. According to the proposed criteria C1 clearly belongs to the newly established genus Thermothelomyces, which contain former thermotolerant Myceliophthora species rather than to the genus Myceliophthora, which remains to include the non-thermotolerant species. As C1 can form ascospores with some other Thermothelomyces (formerly Myceliophthora) strains with opposite mating type, C1 is best classified as Th. heterothallica strain C1, rather than Th. thermophila C1.

It must also be appreciated that the fungal taxonomy was also in constant move in the past, so the current names listed above may be preceded by a variety of older names beyond Myceliophthora thermophila (van Oorschot, 1977. Persoonia 9(3):403), which are now considered synonyms. For example, Thermothelomyces heterothallica (Marin-Felix et al., 2015. Mycologica, 3:619-63), is synonymized with Corynascus heterotchallicus, Thielavia heterothallica (von Klopotek, 1976. Archives of Microbiology 107(2), 223-224), Chrysosporium lucknowense and thermophile (von Klopotek, 1974. Archives of Microbiology 98(1), 365-369) as well as Sporotrichium thermophile (Alpinis 1963. Nova Hedwigia 5:74).

It is further to be explicitly understood that the present invention encompasses any strain containing a ribosomal DNA (rDNA) sequence that shows 99% homology or more to SEQ ID NO:39, and all those strains are considered to be conspecific with Thermothelomyces heterothallica.

Th. heterothallica strain C1 (as Chrysosporium lucknowense strain C1) and mutants derived therefrom were deposited in accordance with the Budapest Treaty with the number VKM F-3500 D, deposit date Aug. 29, 1996.

Particularly, the term Th. heterothallica strain C1 encompass genetically modified sub-strains derived from the wild type strain, which have been mutated, using random or directed approaches, for example, using UV mutagenesis, or by deleting one or more endogenous genes. For example, the C1 strain may refer to a wild type strain modified to delete one or more genes encoding an endogenous protease and/or one or more genes encoding an endogenous chitinase. For example, C1 strains which are encompassed by the present invention include strain UV18-25, deposit No. VKM F-3631 D; strain NG7C-19, deposit No. VKM F-3633 D; and strain UV13-6, deposit No. VKM F-3632 D. Further C1 strain that may be used according to the teachings of the present invention include HC strain UV18-100f deposit No. CBS141147; HC strain UV18-100f deposit No. CBS141143; LC strain W1L#100I deposit No. CBS141153; and LC strain W1L#100I deposit No. CBS141149 and derivatives thereof.

It is to be explicitly understood that the teachings of the present invention encompass mutants, derivatives, progeny, and clones of the Th. heterothallica C1 strains, as long as these derivatives, progeny, and clones, when genetically modified according to the teachings of the present invention are capable of producing at least one of cannabigerolic acid, at least one cannabigerolic acid precursor and/or at least one cannabigerolic acid product according to the teachings of the invention.

It is to be explicitly understood that the term “derivative” with reference to fungal line encompasses any fungal parent line with modifications positively affecting product yield, efficiency, or efficacy, or affecting any trait improving the fungal derivative as a tool to produce at least one of cannabigerolic acid, at least one cannabigerolic acid precursor and/or at least one cannabigerolic acid product. As used herein, the term “progeny” refers to an unmodified descendant from the parent fungal line, such as cell from cell.

Computational models of metabolic networks have been shown to be an effective tool in studying and engineering microbial metabolism of valuable chemicals production. Due to the fast and ongoing development of the computational tools, the accuracy of such models is increased. The inventors of the present invention have used proprietary data of biochemical reactions existing in various species of ascomycetous filamentous fungi to predict the similarity between the exemplified Th. heterothallica of the present invention and these other fungal species with regard to the capability to produce cannabigerolic acids, cannabigerolic acid precursors and products thereof once engineered according to the teachings of the invention. Using these data, five alternative models predicting which biochemical reactions can take a place in cells of a particular species, including validity scores of such prediction have been generated. These models have been further used to assess the degree of similarity between reaction pathways relevant for CBD production. Model simulations (solving linear optimization problems, minimizing and maximizing each flux variables value when CBD yield is maximized), showed which reactions are essential for reaching maximum theoretical yield of CBD. If the range from minimum to maximum flux value does not include zero, the reaction has to carry flux in order to reach the maximum theoretical yield of CBD and is therefore essential for optimal CBD production. As exemplified hereinbelow, the fungal species examined showed highly similar metabolic pathways for producing precursors for CBDA production. These results also support the working assumption of the present invention that a vast variety of filamentous fungi can be equivalently used according to the teachings of the present invention.

The term “cannabinoid” is used herein in its broadest scope and refers to one of a class of diverse chemical compounds that act on a cannabinoid receptor in cells that repress neurotransmitter release in the brain. In particular, the term refers to phytocannabinoids found in Cannabis and some other plants, particularly to phytocannabinoids found in C. sativa and any derivative thereof.

According to certain embodiments, the cannabinoid or derivative thereof is selected from the group consisting of CBDA (cannabidiolic acid), CBD (cannabidiol), CBD-C4 (cannabidiol-C4), CBDP (cannabidiphorol), CBC (cannabichromene, cannabichromenic acid), CBCA (cannabichromenic acid), CBCN (cannabichromanon), CBCT (cannabicitran), CBCTA (cannabicitranic acid), CBCV (cannabichromevarin), CBCVA (cannabichromevarinic acid), CBDM (cannabidiol monomethylether), CBDV (cannabidivarin), CBDVA (cannabidivarinic acid), CBE (cannabielsoin), CBEA-A (cannabielsoic acid A), CBEA-B (cannabielsoic acid B), CBF (cannabifuran), CBG (cannabigerol), cannabigerolic acid, CBGA (cannabigerolic acid), CBGAM (monomethylether), CBGM (cannabigerol monomethyl ether), CBGV (cannabigerovarin), CBGVA (cannabigerovarinic acid), CBL (cannabicyclol), CBLA (cannabicyclolic acid), CBLV (cannabicyclovarin), CBN (cannabinol), CBNA (cannabinolic acid), CBN-C1 (cannabiorcol), CBN-C4 (cannabinol-C4), CBND (cannabinodiol), CBND (cannabinodiol), CBNM (cannabinol methylether), CBR (cannabiripsol), CBT (cannabicitran), CBT (cannabitriol), CBTVE (cannabitriolvarin), CBV (cannabivarin), cis-THC (delta-9-cis-tetrahydrocannabinol), CNB-C2 (cannabinol-C2), DCBF (dehydrocannabifuran), OH-iso-HHCV (3,4,5,6-tetrahydro-7-hydroxy-alpha-alpha-2-trimethyl-9-n-propyl-2,6-methano-2H-1-benzoxocin-5-methanol), OTHC (10-oxo-delta-6a-tetrahydrocannabinol), triOH-THC (trihydroxy-delta-9-tetrahydrocannabinol), Tetrahydrocannabivarin (THCVA), Δ7-cis-iso-tetrahydrocannabivarin, Δ8 -THC (Δ8-trans-tetrahydrocannabinol), tetrahydrocannabinol (THC), Δ8-THCA (Δ8-tetrahydrocannabinolic acid), Δ9-THCA-C1 (Δ9-tetrahydrocannabiorcolic acid), Δ9-tetrahydrocannabinol-C4 (THC-C4), Δ9-THC (Δ9-trans-tetrahydrocannabinol), Δ9-THCA (Δ9-tetrahydrocannabinolic acid), Δ9-THC-C1 (Δ9-tetrahydrocannabiorcol), Δ9-THCV (Δ9-tetrahydrocannabivarin), Δ9-THCVA (Δ9-tetrahydrocannabivarin acid) and tetrahydrocannabiphorol” (THCP).

The terms “olivetolic acid” and “OA” are used herein interchangeably. OA is a member of the class of benzoic acids (2,4-Dihydroxy-6-pentylbenzoic acid) also referred to as olivetolate, olivetolcarboxylic acid, and allazetolcarboxylic acid.

The terms “Olivetol synthase” and “OLS” are used herein interchangeably and refer to 3,5,7-trioxododecanoyl-CoA synthase (EC 2.3.1.206), catalyzing the reaction:

3 malonyl-CoA+hexanoyl-CoA<=>3 CoA+3,5,7-trioxododecanoyl-CoA+3 CO₂.

It is a polyketide synthase catalyzing the first committed step in the cannabinoid biosynthetic pathway of the plant C. sativa.

The terms “olivetolic acid cyclase” and (OAC) are used herein interchangeably and refer to 3,5,7-trioxododecanoyl-CoA<=>CoA+2,4-dihydroxy-6-pentylbenzoate (EC 4.4.1.26) catalyzing the reaction:

3,5,7-trioxododecanoyl-CoA<=>CoA+2,4-dihydroxy-6-pentylbenzoate.

The terms “prenyltransferase”, “aromatic prenyltransferase”, “PT” with reference to enzymes having cannabigerolic acid synthase activity are used herein interchangeably and refer to enzymes capable of prenylation of OA with the monoterpene geranyl pyrophosphate (GPP) to form cannabigerolic acid (CBGA).

The terms “cannabidiolic acid synthase” and “CBDAS” are used herein interchangeably and refer to an enzyme (EC 1.21.3.8) catalyzing the reaction:

Cannabigerolic acid+O₂<=>cannabidiolic acid+H₂O₂.

The enzyme can also convert cannabinerolate to cannabidiolic acid with lower efficiency.

The term “heterologous” as used herein refers to polynucleotide or polypeptide which is not naturally present and/or naturally expressed within a fungus, particularly in Th. heterothallica.

The term “exogenous” as used herein refers to a polynucleotide which is not naturally expressed within the fungus (e.g., heterologous polynucleotide from a different species) or to an endogenous nucleic acid of which overexpression in the fungus is desired. The exogenous polynucleotide may be introduced into the fungus in a stable or transient manner, so as to produce a ribonucleic acid (RNA) molecule and/or a polypeptide molecule. The term “endogenous” as used herein refers to a polynucleotide or polypeptide which is naturally present and/or naturally expressed within a fungus, particularly Th. heterothallica.

The term “overexpression” as used herein refers to an elevated level of gene product (whether nucleic acid or protein), or any metabolite produced as a result of the catalytic activity of a certain overexpressed gene product or a combination of gene products as compared with the expression of the same in the parental strain.

The terms “DNA construct”, expression vector”, “expression construct” and “expression cassette” are used to refer to an artificially assembled or isolated nucleic acid molecule which includes a nucleic acid sequence encoding a protein of interest and which is assembled such that the protein of interest is functionally expressed in a target host cell. An expression vector typically comprises appropriate regulatory sequences operably linked to the nucleic acid sequence encoding the protein of interest. An expression vector may further include a nucleic acid sequence encoding a selection marker.

The terms “polynucleotide”, “nucleic acid sequence”, and “nucleotide sequence” are used herein to refer to polymers of deoxyribonucleotides (DNA), ribonucleotides (RNA), and modified forms thereof in the form of a separate fragment or as a component of a larger construct. A nucleic acid sequence may be a coding sequence, i.e., a sequence that encodes for an end product in the cell, such as a protein. According to certain embodiments of the invention, the protein is an enzyme. According to certain exemplary embodiments, the encoded enzymes include, but are not limited to, OLS, OAC, CBGAS, PT and CBDAS. A nucleic acid sequence may also be a regulatory sequence, such as, for example, a promoter, or a terminator.

The terms “peptide”, “polypeptide” and “protein” are used herein to refer to a polymer of amino acid residues. The term “peptide” typically indicates an amino acid sequence consisting of 2 to 50 amino acids, while “protein” indicates an amino acid sequence consisting of more than 50 amino acid residues.

A sequence (such as, nucleic acid sequence and amino acid sequence) that is “homologous” to a reference sequence refers herein to percent identity between the sequences, where the percent identity is at least 70%, at least 75%, preferably at least 80%, at least 85%, at least 90%, at least 95%, at least 98% at least 99% or at least 99.5%. Each possibility represents a separate embodiment of the present invention. Homologous nucleic acid sequences include variations related to codon usage and degeneration of the genetic code.

Nucleic acid sequences encoding the polypeptides of the present invention may be optimized for expression. Examples of such sequence modifications include, but are not limited to, an altered G/C content to more closely approach that typically found in filamentous fungi, Th. heterothallica being an exemplary species, and the removal of codons atypically found in Th. heterothallica and other fungi commonly referred to as codon optimization.

The phrase “codon optimization” refers to the selection of appropriate DNA nucleotides for use within a structural gene or fragment thereof that approaches codon usage within the organism of interest, and/or to a process of modifying a nucleic acid sequence for enhanced expression in the host cells of interest by replacing at least one codon (e.g., one or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Therefore, an optimized gene or nucleic acid sequence refers to a gene in which the nucleotide sequence of a native or naturally occurring gene has been modified in order to utilize statistically-preferred or statistically-favored codons within the organism. The present invention explicitly encompasses polynucleotides encoding the enzyme of interest as disclosed herein which are codon optimized for expression in Th. heterothallica and other ascomycetes filamentous fungi.

Sequence identity may be determined using a nucleotide/amino acid sequence comparison algorithm, as known in the art.

The term “coding sequence” is used herein to refer to a sequence of nucleotide starting with a start codon (ATG) containing any number of codons excluding stop codons, and a stop codon (TAA, TGA, TAA), which code for a functional polypeptide.

Any coding sequence, or amino acid sequence listed herein also encompasses truncated sequences, which are missing 1, 2, 3, 4, 5, 10, 15, 20, 25, 50, or more codons or amino acids from any part of the sequence. Truncated versions of coding sequences or amino sequences can be identified using nucleotide/amino acid sequence comparison algorithm, as known in the art.

Any coding sequence, or amino acid sequence listed herein also encompasses fused sequences, which contain besides the coding sequence provided herein, or a truncation of that sequence as defined above, other sequences. The fused sequences can be sequences as disclosed herein and other sequences. Fused coding sequences or amino sequences can be identified using nucleotide/amino acid sequence comparison algorithm, as known in the art.

The terms “mature protein” or “a protein lacking a signal peptide” are used herein interchangeably to refer to a version of a protein, where the signal sequence, used by the cell to direct the protein of interest to membrane organelles such as endoplasmic reticulum (ER), Golgi, vacuoles or alike, are replaced with a single methionine enabling translation initiation. Thus, the resulting peptide will localize into the cytoplasm, and will exert its enzymatic activity in that cellular compartment. Signal peptides can be recognized using signal peptide prediction algorithms as known by the art. For example, the various versions of the SignalP service at www.cbs.dtu.dk can be used to identify such sequences. A skilled artisan thus can generate a mature protein version lacking a signal peptide, or a coding sequence encoding such mature protein by any method as is known in the art.

The term “regulatory sequences” refer to DNA sequences which control the expression (transcription) of coding sequences, such as promoters, enhancers and terminators.

The term “promoter” is directed to a regulatory DNA sequence which controls or directs the transcription of another DNA sequence in vivo or in vitro. Usually, the promoter is located in the 5′ region (that is, precedes, located upstream) of the transcribed sequence. Promoters may be derived in their entirety from a native source, or be composed of different elements derived from different promoters found in nature, or even comprise synthetic nucleotide segments. Promoters can be constitutive (i.e. promoter activation is not regulated by an inducing agent and hence rate of transcription is constant), or inducible (i.e., promoter activation is regulated by an inducing agent or environmental condition). Promoters may also restrict transcription to a certain developmental stage or to a certain morphologically distinct part of the organism. In most cases the exact boundaries of regulatory sequences have not been completely defined, and in some cases, cannot be completely defined, and thus DNA sequences of some variation may have identical promoter activity.

The term “terminator” is directed to another regulatory DNA sequence which regulates transcription termination. A terminator sequence is operably linked to the 3′ terminus of the nucleic acid sequence to be transcribed.

The terms “C1 promoter” and “C1 terminator” indicate promoter and terminator sequences suitable for use in C1, i.e., capable of directing gene expression in C1. The practical method for definition of these regulatory sequences is described under the examples.

Suitable homogenous or heterogeneous promoters and terminators are listed under the examples. However, as known to the skilled artisan, the choice of promoters and terminators may not be critical, and similar results can be obtained with a variety of promoters and terminators providing similar or identical gene expression.

The term “operably linked” means that a selected nucleic acid sequence is in proximity with a regulatory element (promoter, enhancer and/or terminator) to allow the regulatory element to regulate expression of the selected nucleic acid sequence.

The present invention discloses the production of substantially pure cannabigerolic acid (CBGA), products and derivatives thereof, using genetically modified strains of Th. heterothallica C1. As described hereinabove, filamentous fungi of other species sharing endogenous similar pathways of precursor production can be also used.

In the plant C. sativa production of CBGA is an initial step in the production of many cannabinoids. Once CBGA is produced, a single additional enzymatic step is required to turn CBGA into many other cannabinoids (CBDA, THCA, CBCA, etc.). The present invention is aimed, according to certain embodiments, at producing cannabidiolic acid (CBDA), from which cannabidiol (CBD) is produced through non-enzymatic decarboxylation, and/or at producing tetrahydrocannabinolic acid (THCA), from which tetrahydrocannabinol (THC) is produced through non-enzymatic decarboxylation, and derivatives thereof. The resulting CBD and or THC are highly pure and can be used in the pharmaceutical/nutraceutical industry to treat a wide range of health issues. Furthermore, the produced cannabinoids can be used for the production of any derivative as is currently known and as will be known in the Art.

The present invention discloses the production of substantially pure cannabigerolic acid (CBGA), derivatives and products thereof using genetically modified strains of Th. heterothallica C1 and similar fungi, particularly the production of CBDA, CBD, THCA, THC and derivatives thereof.

An advantage of using the filamentous fungi, particularly Th. heterothallica for the production of cannabinoids is the natural capability of these fungi to produce elevated levels of precursor molecules geranyl pyrophosphate (GPP), and hexanoyl-CoA as compared with yeasts, hitherto known to be used in fermentation systems for production of cannabinoids. Specifically, Th. heterothallica C 1 encodes within its genome a phosphoketolase gene not present in S. cerevisiae.

The following reactions can be naturally (i.e. without the need to transform heterologous genes) carried out in Th. heterothallica C1, as well as all investigated ascomycetous filamentous fungi, such as Aspergillus nidulans, Trichoderma reesei, Rasamsonia emersonii and several Penicillium species, but not in S. cerevisiae:

The reaction carried out by fructose-6-phosphate phosphoketolase (EC4.1.2.22).

D-Xylulose 5-phosphate+orthophosphate<=>Acetyl orthophosphate+D-glyceraldehyde 3-phosphate+H₂O.

This reaction is carried out by acylphosphatase (EC:3.6.1.7).

Acetyl orthophosphate+H₂O<=>acetate+orthophosphate

The presence of the said enzymes offers increased cytoplasmic Acetyl-CoA productions, which leads to increased geranyl pyrophosphate (GPP) production, which is a direct precursor in the cannabinoid production pathway, and therefore leads to higher production of CBGA and products thereof.

Th. heterothallica also comprise biosynthetic pathway(s) for synthesizing Hexanoyl-CoA from Hexanoic acid (a simple fatty acid). GPP and Hexanoyl-CoA are necessary precursor compounds in the production of CBGA.

Th. heterothallica naturally produces butyryl-CoA as a degradation product of β-oxidation, intermediate of fatty acid synthesis or produced via following enzyme reactions: 2 acetyl-CoA to acetoacetyl-CoA+CoA with acetoacetyl-CoA thiolase followed by acetoacetyl-CoA to 3-hydroxybutyryl-CoA with 3-hydroxybutyryl-CoA dehydrogenase followed by 3-hydroxybutyryl-CoA to crotonyl-CoA with 3-hydroxybutyryl-CoA dehydratase followed by crotonyl-CoA to butyryl-CoA with butyryl-CoA dehydrogenase.

Butyryl-CoA forms part of Th. heterothallica and other filamentous fungi fatty acids biosynthesis pathway in parallel to the production of hexanoyl CoA. Butyryl-CoA can be used for the production of divarinolic acid by the same enzymes converting hexanoyl CoA to olivetolic acid, i.e. OLS and OAC. Thereafter, divarinolic acid can be used for the synthesis of cannabigerovarinic acid (CBGVA), again by the same prenyltransferase (PT) enzyme that coverts olivetolic acid to cannabigerolic acid (CBGA). CBGVA is the precursor for the production of cannabidivarinic acid (CBDVA) by the cannabidiolic acid synthase enzyme. CBDVA, like CBDA, can further be converted to cannabidivarin (CBDV) by chemical decarboxylation. The synthesis of CBGVA can be performed in vivo within the filamentous fungi or in vitro.

The present invention thus explicitly encompasses transgenic filamentous fungi producing CBGVA and/or CBDVA. Thus, according to certain embodiments, the production of CBGA, CBGVA or CBDVA or CBDA in this fungus requires only the following biosynthetic steps: Conversion of CoA ester with C4 to C8 aliphatic side chains, e.g. hexanoyl-CoA to olivetolic acid (OA) or butyryl CoA to divarinolic acid. Polyketides are formed in two-steps reaction by the polyketide synthase olivetol synthase and further cyclization by olivetolic acid cyclase to form OA or divarinoic acid, respectively. Thereafter, OA or divarinolic acid is prenylated with the monoterpene geranyl pyrophosphate (GPP) to cannabigerolic acid (CBGA) or cannabigerovarinic acid (CBGVA) by an aromatic prenyltransferase (PT).

For the formation of cannabidiol (CBD) or cannabidivarin (CBDV), the fungus further comprises CBDA synthase (CBDAS), cyclizing cannabigerolic acid or cannabigerovarinic acid to CBDA or CBDVA, respectively. The last step from cannabidiolic acid or cannabidivarinic acid to cannabidiol or cannabidivarin is carried out with non-enzymatic decarboxylation (Zirpel et. al. 2017. J Biotech. 259:204-212).

For the formation of tetrahydrocannabinol (THC) or tetrahydrocannabivarin (THCV), the fungus further comprises tetrahydrocannabinolic acid (THCA) synthase catalyzing the formation of THCA or THCVA from cannabigerolic acid (CBGA) or cannabigerovarinic acid (CBGVA). Non-enzymatic decarboxylation of THCA or THCVA forms THC or THCV, respectively.

According to certain currently exemplary embodiments, the polynucleotides of the present invention are designed based on the amino acid sequence of the enzyme to be produced employing a codon usage of a filamentous fungus. According to certain embodiments, the filamentous fungus belongs to the group Pezizomycotina. According to some embodiments, the filamentous fungus belongs to a group selected from the group consisting of Sordariales, Hypocreales Onygenales, and Eurotiales including genera and species as described in the “definition” section hereinabove.

According to certain exemplary embodiments, the fungus is Th. heterothallica. According to certain currently exemplary embodiments, the fungus is Th. heterothallica C1. According to these embodiments, the polynucleotides encoding enzymes according to the teachings of the present invention are optimized for expression in this fungus.

According to certain exemplary embodiments, the Th. heterothallica C1 strain is a derivative of strain UV18-25.

According to certain embodiments, the exogenous polynucleotide is endogenous to the fungus, particularly to Th. heterothallica C1. According to certain embodiments, the exogenous polynucleotide is heterologous to the fungus, particularly to Th. heterothallica C1.

According to certain embodiments, the OLS comprises an amino acid sequence at least 75% homologous to the amino acid sequence of C. sativa OLS. According to certain exemplary embodiments, the C. sativa OLS comprises the amino acid sequence set forth in SEQ ID NO:1. According to certain embodiments, the coding sequence of OLS is codon optimized to be used in Th. heterothallica C1, said coding sequence comprising the nucleic acid sequence set forth in SEQ ID NO:2.

According to certain embodiments, the OAC comprises an amino acid sequence at least 75% homologous to the amino acid sequence of C. sativa OAC. According to certain exemplary embodiments, the C. sativa OAC comprises the amino acid sequence set forth in SEQ ID NO:3. According to certain embodiments, the coding sequence of OAC is codon optimized to be used in Th. heterothallica C1, said coding sequence comprising the nucleic acid sequence set forth in SEQ ID NO:4.

According to certain embodiments, the Prenyltransferase (PT) comprises an amino acid sequence at least 75% homologous to the amino acid sequence of C. sativa CBGAS PT1 or CBGAS PT4 or Streptomyces sp. CL190 NphB. According to certain exemplary embodiments, the C. sativa CBGAS PT1 comprises the amino acid sequence set forth in SEQ ID NO:5. According to certain embodiments, the coding sequence of C. sativa CBGAS PT1 is codon optimized to be used in Th. heterothallica C1, said coding sequence comprising the nucleic acid sequence set forth in SEQ ID NO:6. According to certain exemplary embodiments, the C. sativa CBGAS PT4 comprises the amino acid sequence set forth in SEQ ID NO:7. According to certain embodiments, the coding sequence of C. sativa CBGAS PT4 is codon optimized to be used in Th. heterothallica C1, said coding sequence comprising the nucleic acid sequence set forth in SEQ ID NO:8. According to certain currently exemplary embodiments, the codon-usage optimized polynucleotide encodes a mature protein without a signal peptide (PT4t), said polynucleotide comprises the nucleic acid sequence set forth in SEQ ID NO:88. According to certain exemplary embodiments, the Streptomyces sp. CL190 NphB comprises the amino acid sequence set forth in SEQ ID NO:9. According to certain embodiments, the coding sequence of Streptomyces sp. CL190 NphB is codon optimized to be used in Th. heterothallica C1, said coding sequence comprising the nucleic acid sequence set forth in SEQ ID NO:10.

According to certain exemplary embodiments, the Prenyltransferase is PT4. According these embodiments, the PT4 is encoded by a polynucleotide comprising the nucleic acid sequence set forth in SEQ ID NO:8 or a part thereof. According to some embodiments, the PT4 is a mature protein lacking a signal peptide (PT4t), said PT4t comprises the amino acid sequence set forth in SEQ ID NO:89 and is encoded by the nucleic acid sequence set forth in SEQ ID NO:88.

According to certain embodiments, the CBDAS comprises an amino acid sequence at least 75% homologous to the amino acid sequence of C. sativa CBDAS. According to certain exemplary embodiments, the C. sativa CBDAS either possessing or lacking a signal peptide. According to certain embodiments, the C. sativa CBDAS comprises the amino acid sequence set forth in SEQ ID NO:11, said protein comprises a signal peptide. According to certain currently exemplary embodiments, the CBDAS is a mature protein lacking a signal peptide, said mature protein comprises the amino acid sequence set forth in SEQ ID NO:90. According to certain embodiments, the coding sequence of C. sativa CBDAS is codon optimized to be used in Th. heterothallica C1, said coding sequence comprising the nucleic acid sequence set forth in SEQ ID NO:12. According to certain currently exemplary embodiments, the codon optimized polynucleotide encoding C. sativa CBDAS is lacking the nucleic acid sequence encoding the signal peptide, said polynucleotide having the nucleic acid sequence set forth in Seq ID NO:91.

According to certain embodiments, the THCAS comprises an amino acid sequence at least 75% homologous to the amino acid sequence of C. sativa THCAS. According to certain exemplary embodiments, the C. sativa THCAS protein either possessing or lacking a signal peptide. According to certain exemplary embodiments, the C. sativa THCAS mature protein comprises the amino acid sequence set forth in SEQ ID NO:13, said protein comprises a signal peptide having amino acids 1-28 of SEQ ID NO:13.

According to certain embodiments, the hexanoate synthase is homologous to hexanoate synthase of Aspergillus parasiticus strain SU-1. According to certain embodiments, the hexanoate synthase comprises one unit at least 75% homologous to the amino acid sequence of A. parasiticus strain SU-1 hexanoate synthase alpha subunit (HexA) and another unit at least 75% homologous to the amino acid sequence of A. parasiticus strain SU-1 hexanoate synthase beta subunit (HexB). According to certain exemplary embodiments, the HexA subunit comprises the amino acid sequence set forth in SEQ ID NO:15. According to certain embodiments, the coding sequence of HexA is codon optimized to be used in Th. heterothallica C1, said coding sequence comprising the nucleic acid sequence set forth in SEQ ID NO:16. According to certain exemplary embodiments, the HexB subunit comprises the amino acid sequence set forth in SEQ ID NO:17. According to certain embodiments, the coding sequence of HexA is codon optimized to be used in Th. heterothallica C1, said coding sequence comprising the nucleic acid sequence set forth in SEQ ID NO:18.

According to certain exemplary embodiments, the acyl-activating enzyme comprises an amino acid sequence at least 75% homologous the amino acid sequence of any one of C. sativa acyl-activating enzyme 1 (AAE1) and C. sativa acyl-activating enzyme 3 (AAE3). Each possibility represents a separate embodiment of the present invention. According to certain exemplary embodiments, the AEE1 comprises the amino acid sequence set forth in SEQ ID NO:19 and the AEE3 comprises the amino acid sequence set forth in SEQ ID NO:21. According to certain embodiments, the coding sequence of AAE1 is codon optimized to be used in Th. heterothallica C1, said coding sequence comprising the nucleic acid sequence set forth in SEQ ID NO:20. According to certain embodiments, the coding sequence of AAE1 is codon optimized to be used in Th. heterothallica C1, said coding sequence comprising the nucleic acid sequence set forth in SEQ ID NO:22. According to currently exemplary embodiments, the acyl-activating enzyme comprises the amino acid sequence of C. sativa acyl-activating enzyme 1 (AAE1).

According to certain exemplary embodiments, the GPP synthetase enzyme comprises an amino acid sequence at least 75% homologous to the amino acid sequence of any one of Th. heterothallica GPPS, S. cerevisiae ERG20 (K197E) FPPS or S. cerevisiae ERG20 (F96W-N127W) FPPS. Each possibility represents a separate embodiment of the present invention. According to certain exemplary embodiments, the Th. heterothallica GPPS comprises the amino acid sequence set forth in SEQ ID NO:23, S. cerevisiae ERG20 (K197E) FPPS comprises the amino acid sequence set forth in SEQ ID NO:25, S. cerevisiae ERG20 (F96W-N127W) FPPS comprises the amino acid sequence set forth in SEQ ID NO:27. According to certain embodiments, the coding sequence of Th. heterothallica GPPS is the native Th. heterothallica sequence set forth in SEQ ID NO:24, the coding sequence of S. cerevisiae ERG20 (K197E) FPPS is codon optimized to be used in Th. heterothallica C1, said coding sequence comprising the nucleic acid sequence set forth in SEQ ID NO:26, and the coding sequence of S. cerevisiae ERG20 (F96W-N127W) FPPS is codon optimized to be used in Th. heterothallica C1, said coding sequence comprising the nucleic acid sequence set forth in SEQ ID NO:28. Each possibility represents a separate embodiment of the present invention.

According to certain exemplary embodiments, the HMG CoA reductase enzyme comprises an amino acid sequence at least 75% homologous the amino acid sequence of S. cerevisiae truncated HMG1. According to certain exemplary embodiments, the S. cerevisiae truncated HMG1 comprises the amino acid sequence set forth in SEQ ID NO:29. According to certain embodiments, the coding sequence of S. cerevisiae truncated HMG1 is codon optimized to be used in Th. heterothallica C1, said coding sequence comprising the nucleic acid sequence set forth in SEQ ID NO:30.

According to certain exemplary embodiments the Fructose-6-phosphate phosphoketolase is Th. heterothallica Fructose-6-phosphate phosphoketolase 1, or Th. heterothallica Fructose-6-phosphate phosphoketolase 2. According to certain exemplary embodiments, the Fructose-6-phosphate phosphoketolase enzyme comprises an amino acid sequence at least 75% homologous the amino acid sequence of Th. heterothallica C1 Fructose-6-phosphate phosphoketolase 1 set forth in SEQ ID NO:31. According to certain exemplary embodiments, the Fructose-6-phosphate phosphoketolase enzyme comprises an amino acid sequence at least 75% homologous the amino acid sequence of Th. heterothallica C1 Fructose-6-phosphate phosphoketolase 2 set forth in SEQ ID NO:33. According to certain embodiments, the coding sequence of Th. heterothallica C1 Fructose-6-phosphate phosphoketolase 2 is the native Th. heterothallica coding sequence set forth in SEQ ID NO:32. According to certain embodiments, the coding sequence of Th. heterothallica C1 Fructose-6-phosphate phosphoketolase 2 is the native Th. heterothallica coding sequence set forth in SEQ ID NO:34.

According to certain exemplary embodiments the acyl phosphatase is Th. heterothallica acyl phosphatase. According to certain exemplary embodiments, the acyl phosphatase enzyme comprises an amino acid sequence at least 75% homologous the amino acid sequence of Th. heterothallica acyl phosphatase set forth in SEQ ID NO:35. According to certain embodiments, the coding sequence of Th. heterothallica C1 acyl phosphatase is the native Th. heterothallica coding sequence set forth in SEQ ID NO:36.

The polynucleotides encoding each of the enzymes may form part of one or more DNA constructs and/or expression vectors. According to certain embodiments, each of the polynucleotide forms part of a separate DNA construct/vector. According to other embodiments, part or all the polynucleotides are present within the same DNA construct/expression vector. This means that genes may be introduced one by one, or several of them may also be introduced to the transformed fungi at one time.

The DNA constructs or expression vector or plurality of same each comprises regulatory elements controlling the transcription of the polynucleotides within the at least one fungus cell. The regulatory element can be a regulatory element endogenous to the fungus, particularly to Th. heterothallica C1 or exogenous to the fungus.

According to certain embodiments, the regulatory element is selected from the group consisting of a 5′ regulatory element (collectively referred to as promoter), and 3′ regulatory element (collectively referred to as terminator), even though these nucleotide sequences may contain additional regulatory elements not classified as promoter or terminator sequences in the strict sense.

According to certain embodiments, the DNA construct or expression vector comprises at least one promoter operably linked to at least one polynucleotide containing a coding sequence, operably linked to at least one terminator. According to certain embodiments, the promoter is endogenous promoter of the fungus, particularly to Th. heterothallica. According to additional or alternative embodiments, the promoter is heterologous to the fungus, particularly to Th. heterothallica. According to certain embodiments, the terminator is endogenous terminator of the fungus, particularly to Th. heterothallica. According to additional or alternative embodiments, the terminator is heterologous to the fungus, particularly to Th. heterothallica.

According to certain exemplary embodiments, the DNA constructs contain synthetic regulatory elements called as “synthetic expression system” (SES) essentially as described in International (PCT) Application Publication No. WO 2017/144777.

According to certain embodiments, the one or more polynucleotides is stably integrated into at least one chromosomal locus of the at least one cell of the genetically modified fungus. According to certain embodiments, the one or more polynucleotides is/are stably integrated into one or more defined sites on the fungal chromosomes. According to certain embodiments, the one or more polynucleotides is/are stably integrated into random sites of the chromosome. According to certain embodiments, the polynucleotides may be incorporated in targeted or random fashion as 1, 2, or more copies to 1, 2 or more chromosomal loci.

According to certain alternative embodiments, the one or more polynucleotides is transiently expressed using extrachromosomal expression vectors as is known to a person skilled in the art.

According to certain exemplary embodiments the Th. heterothallica ku70 homologous gene set forth in SEQ ID NO:37 is knocked out by preferentially eliminating the full coding sequence of the ku70 gene as known in the art. The inactivation of the ku70 gene enhances the percentage of targeted transformations as known in the art.

According to certain exemplary embodiments the Th. heterothallica ant1 gene set forth in SEQ ID NO:38 is knocked out by preferentially eliminating the full coding sequence of the ant1 gene as known in the art. The inactivation of the ant1 gene eliminates a metabolic pathway that acts against the accumulation of cannabinoid precursors. According to certain additional embodiments the same strategy can be used to inactivate other metabolic pathways that interfere with the accumulation of cannabinoid precursors, or otherwise interfere with the accumulation of the desired product or products.

According to certain exemplary embodiments the genes encoding the at least one enzyme required for cannabinoid production (selected from the group consisting of SEQ ID NOs:1, 3, 5, 7, 9, 11, and 13) are targeted to the ant1 locus. According to additional embodiments the at least one gene required for cannabinoid production is targeted to hot spots of the genome, different from the ant1 locus allowing high expression as is known in the art.

According to certain exemplary embodiments the at least one gene encoding an enzyme required for enhancing cannabinoid production (selected from the group consisting of SEQ ID NOs:15 and 17, 19, 21, 23, 25, 27, 29, 31, 33, and 35) is targeted to the ant1 locus. According to additional embodiments the at least one gene required for enhancing cannabinoid production are targeted to hot spots of the genome, different from the ant1 locus allowing high expression as is known in the art.

According to certain embodiments, culturing of the genetically modified fungus in a suitable medium provides for synthesis of the cannabigerolic acid, cannabigerolic acid precursor and/or cannabigerolic acid product, and/or derivatives thereof in an increased amount compared to the amount produced in a corresponding unmodified fungus cultured under similar conditions.

According to certain embodiments, culturing of the genetically modified fungus in a suitable medium provides for a source of cell extract, enzyme extract or purified enzyme, which enables bioconversion of cannabigerolic acid, cannabigerolic acid precursor and/or cannabigerolic acid product, and/or derivatives thereof in an increased amount compared to the amount produced similarly in a corresponding unmodified fungus cultured under similar conditions.

According to certain embodiments, the corresponding unmodified fungus is of the same species of the genetically modified fungus. According to some embodiments, the corresponding fungus is isogenic to the genetically modified fungus.

According to certain embodiments, the cannabigerolic acid precursor is selected from the group consisting of hexanoic acid, olivetolic acid, GPP, derivatives thereof and any combination thereof. Each possibility represents a separate embodiment of the present invention.

Cannabigerolic acid is the precursor of a large number of cannabinoids. The genetically modified fungi of the present invention can thus be used for the production of all such cannabinoids and derivatives thereof.

According to certain exemplary embodiments, the present invention provides a genetically modified Th. heterothallica C1 fungus that enables producing cannabigerolic acid and derivatives thereof. According to these embodiments, such genetically modified Th. heterothallica C1 fungus comprises at least one cell comprising (i) at least one heterologous polynucleotide encoding olivetol synthase (OLS); (ii) at least one heterologous polynucleotide encoding olivetolic acid cyclase (OAC); and (iii) at least one heterologous polynucleotide encoding prenyltransferase (PT) having cannabigerolic acid synthase (CBGAS) activity.

According to certain exemplary embodiments the present invention provides a genetically modified Th. heterothallica C1 fungus that enables producing CBDA and CBD and derivatives thereof. According to these embodiments, such genetically modified Th. heterothallica C1 fungus comprises at least one cell comprising (i) at least one heterologous polynucleotide encoding olivetol synthase (OLS); (ii) at least one heterologous polynucleotide encoding olivetolic acid cyclase (OAC); and (iii) at least one heterologous polynucleotide encoding cannabigerolic acid synthase (CBGAS) and/or prenyltransferase (PT); (iv) at least one heterologous polynucleotide encoding cannabidiolic acid synthase (CBDAS).

According to certain exemplary embodiments the present invention provides a genetically modified Th. heterothallica C1 fungus that enables producing THCA and THC and derivatives thereof. According to certain exemplary embodiments, the tetrahydrocannabinolic acid product is tetrahydrocannabinol (THC) and derivatives thereof. According to some embodiments, the THC is selected from the group consisting of Δ9-trans-tetrahydrocannabinolic acid (Δ9-THC), Δ8-trans-tetrahydrocannabinol (Δ8-THC), derivatives thereof and any combination thereof. According to these embodiments, such genetically modified Th. heterothallica C1 fungus comprises at least one cell comprising (i) at least one heterologous polynucleotide encoding olivetol synthase (OLS); (ii) at least one heterologous polynucleotide encoding olivetolic acid cyclase (OAC); and (iii) at least one heterologous polynucleotide encoding prenyltransferase (PT) having cannabigerolic acid synthase (CBGAS) activity; and (iv) at least one heterologous polynucleotide encoding tetrahydrocannabinolic acid synthase (THCAS).

According to certain exemplary embodiments the present invention provides a genetically modified Th. heterothallica C1 fungus that enables producing commercially relevant amounts of CBDA and CBD and derivatives thereof, or THCA and THC and derivatives thereof. According to these embodiments, such genetically modified Th. heterothallica C1 fungus comprises at least one cell comprising in addition to (i) at least one heterologous polynucleotide encoding olivetol synthase (OLS); (ii) at least one heterologous polynucleotide encoding olivetolic acid cyclase (OAC); (iii) at least one heterologous polynucleotide encoding prenyltransferase (PT) having cannabigerolic acid synthase (CBGAS) activity; at least one of (a) at least one heterologous polynucleotide encoding cannabidiolic acid synthase (CBDAS) and (b) at least one heterologous polynucleotide encoding tetrahydrocannabinolic acid synthase (THCAS).

According to certain exemplary embodiments, the above-described C1 fungus further comprises at least one heterologous polynucleotide encoding HexA/HexB, and/or AAE1, and/or AAE3, and/or GPPS and or FPPS (K197E) and/or FPPS (F96W-N127W) and/or Fructose-6-phosphate phosphoketolase 1 and/or Fructose-6-phosphate phosphoketolase 2 and/or acylphosphatase, as defined hereinabove.

According to certain embodiments, a suitable medium for culturing the genetically modified fungi comprises a carbon source selected from the group consisting of glucose, sucrose, xylose, arabinose, galactose, fructose, lactose, cellobiose, and glycerol. According to some embodiments, the carbon source is provided from waste of ethanol production or other bioproduction from starch, sugar beet and sugar cane such as molasses comprising fermentable sugars, starch, lignocellulosic biomass comprising polymeric carbohydrates such as cellulose and hemicellulose.

According to certain currently exemplary embodiments, the fungus is Th. heterothallica C1. According to certain embodiments, the strain of Th. heterothallica C1 is selected from the group consisting of strain UV18-25, deposit No. VKM F-3631 D; strain NG7C-19, deposit No. VKM F-3633 D; and strain UV13-6, deposit no. VKM F-3632 D. Additional strains that may be used are HC strain UV18-100f deposit No. CBS141147; HC strain UV18-100f deposit No. CBS141143; LC strain W1L#100I deposit No. CBS141153; and LC strain W1L#100I deposit No. CBS141149 and derivatives thereof. Each possibility represents a separate embodiment of the present invention.

According to another aspect, the present invention provides a method for producing a fungus capable of producing cannabigerolic acid and/or cannabigerovarinic acid, at least one cannabigerolic acid and/or cannabigerovarinic acid precursor and/or at least one cannabigerolic acid and/or cannabigerovarinic acid product, and derivatives thereof, the method comprising transforming at least one cell of the fungus with at least one of (i) at least one heterologous polynucleotides encoding olivetol synthase (OLS); (ii) at least one heterologous polynucleotides encoding olivetolic acid cyclase (OAC); (iii) at least one heterologous polynucleotides encoding prenyltransferase (PT)) having cannabigerolic acid synthase (CBGAS) activity; (iv) at least one heterologous polynucleotides encoding cannabidiolic acid synthase (CBDAS); and (v) at least one heterologous polynucleotide encoding tetrahydrocannabinolic acid synthase (THCAS) to produce genetically modified fungus capable of producing cannabigerolic acid, cannabigerolic acid precursors, products thereof and derivatives thereof.

According to certain embodiments, the method further comprises transforming the at least one cell with at least one polynucleotide encoding hexanoate synthase and at least one polynucleotide encoding acyl-activating enzyme.

According to certain additional or alternative embodiments, the method further comprises modulating the expression and/or activity of at least one endogenous enzyme of the fungus fatty acid pathway.

According to yet additional embodiments, the method further comprises transforming the at least one cell with at least one polynucleotide encoding geranyl-pyrophosphate synthase (GPPS).

According to certain additional or alternative embodiments, the method further comprises transforming the at least one cell with at least one polynucleotide encoding a modified farnesyl pyrophosphate synthase (FPPS) having GPPS activity.

According to certain additional or alternative embodiments, the method further comprises overexpressing at least one endogenous polynucleotide selected from the group consisting of a polynucleotide encoding fructose-6-phosphate phosphoketolase; a polynucleotide encoding acylphosphatase; and a combination thereof

According to certain exemplary embodiments, the fructose-6-phosphate phosphoketolase comprises an amino acid sequence at least 75% homologous to the amino acid sequence set forth in any one of SEQ ID NO:31, and SEQ ID NO:33. According to further certain exemplary embodiments, the acylphosphatase comprises an amino acid sequence at least 75% homologous to the amino acids sequence as set forth SEQ ID NO:35.

According to certain embodiments, the genetically modified fungus produces cannabigerolic acid or cannabigerolic acid derivatives, cannabigerolic acid precursors or cannabigerolic acid precursor derivatives; and/or cannabigerolic acid products or cannabigerolic acid product derivatives in an elevated amount compared to the amount produced by a corresponding fungus not transformed with the polynucleotides.

According to certain embodiments, the genetically modified fungus produces cannabigerovarinic acid or cannabigerovarinic acid derivatives, cannabigerovarinic acid precursors or cannabigerovarinic acid precursor derivatives; and/or cannabigerovarinic acid products or cannabigerovarinic acid product derivatives in an elevated amount compared to the amount produced by a corresponding fungus not transformed with the polynucleotides.

According to certain embodiments, the cannabigerolic acid precursor is selected from the group consisting of hexanoic acid, olivetolic acid, GPP, and a combination thereof. Each possibility represents a separate embodiment of the present invention.

According to certain embodiments, the cannabigerolic acid product is selected from the group consisting of cannabidiolic acid (CBDA), cannabidiol (CBD), tetrahydrocannabinolic acid (THCA), tetrahydrocannabinol (THC), derivatives thereof, and any combination thereof.

According to certain embodiments, the cannabigerovarinic acid product is selected from the group consisting cannabidivarinic acid (CBDVA), cannabidivarin (CBDV), derivatives thereof, and any combination thereof.

Any method as is known in the art for transforming filamentous fungi with at least one polynucleotide can be used according to the teachings of the present invention.

The fungus and the polynucleotides are as described hereinabove.

According to yet another aspect, the present invention provides a method of producing at least one of cannabigerolic acid, cannabigerolic acid precursors, cannabigerolic acid products, derivative thereof, and any combination thereof, the method comprising culturing the genetically modified fungus, particularly Th. heterothallica C1 fungi of the present invention in a suitable medium; and recovering the produced products.

According to certain embodiments, the medium comprises a carbon source selected from the group consisting of glucose, sucrose, xylose, arabinose, galactose, fructose, lactose, cellobiose, and glycerol. According to certain embodiments the carbon source is waste obtained from ethanol production or other bioproduction from starch, sugar beet and sugar cane such as molasses comprising fermentable sugars, starch, lignocellulosic biomass comprising polymeric carbohydrates such as cellulose and hemicellulose.

According to certain embodiments, the cannabigerolic acid, cannabigerolic acid precursors, cannabigerolic acid products and/or derivatives thereof are extracted from the fungal mass. Any method as is known in the art for extracting cannabinoids from vegetative tissues can be used. According to additional or alternative embodiments, the cannabigerolic acid, precursors, products and/or derivatives thereof are recovered from the fungi growth medium.

According to certain embodiments, the cannabigerolic acid product is selected from the group consisting of cannabidiolic acid (CBDA), cannabidiol (CBD). tetrahydrocannabinolic acid (THCA), tetrahydrocannabinol (THC), derivatives thereof, and any combination thereof. According to certain exemplary embodiments, the cannabigerolic acid product is CBD. According to some embodiments, the CBD is a pharmaceutical grade CBD.

According to certain exemplary embodiments, the cannabigerolic acid product is THC. According to some embodiments, the THC is a pharmaceutical grade THC.

According to a further aspect, the present invention provides cannabigerolic acid, cannabigerolic acid precursor, cannabigerolic acid product, and/or derivatives thereof produced by the genetically modified fungus, particularly the genetically modified Th. heterothallica C1 of the present invention.

According to certain embodiments, the cannabigerolic acid product is cannabidiol (CBD).

According to certain embodiments, the cannabigerolic acid product is tetrahydrocannabinol (THC).

According to certain embodiments, the cannabigerolic acid, cannabigerolic acid precursor, and/or cannabigerolic acid product is of a pharmaceutical grade.

According to certain embodiments, the cannabigerolic acid product is a pharmaceutical grade cannabidiol (CBD).

According to certain embodiments, the cannabigerolic acid product is a pharmaceutical grade tetrahydrocannabinol (THC).

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Methods

### Cultivation Conditions

Th. heterothallica was cultivated in complete medium that contains 35 mM (NH₄)₂SO₄, 7 mM NaCl, 55 mM KH₂PO₄, 0.5% Yeast extract, 0.1% Casamino acids (BD Bacto™ Casamino Acids), 10 mM Uracil, 1% glucose, 2-mM MgSO₄, 10 mM uridine, 174 μM EDTA, 76 μM ZnSO₄.7H₂O, 178 μM H₃BO₃, 25-μM MnSO₄.H₂O, 18 μM FeSO₄.7H₂O, 7.1 mM CoCl₂.6H₂O, 6.4 μM CuSO₄.5H₂O, 6.2 μM Na₂MoO₄.2H₂O, pH 6.5. For small scale, cultivation was performed in 3.5 ml volume in 24-well plates sealed with an adhesive breathable rayon film, in a humidified shaker at 35° C. with 800 rpm shaking.

Metabolite Extraction from Th. Heterothallica Cultures

Two alternative methods, cold methanol and ethyl acetate extraction, were used to extract metabolites from Th. heterothallica.

Methanol extraction was carried out as follows: 1 ml sample containing mycelia and liquid culture medium was added into 4 ml −80° C. cold methanol:H₂O 2.5:1.5 containing internal standards (final methanol concentration 50%), mixed by vortexing and incubated in −80° C. for at least 1 h. Samples were mixed by vortexing and centrifuged at 7800 rpm at 4° C. for 10-15 min. The supernatants were collected for analysis.

Ethyl acetate extraction was carried as follows: 500 μl ethyl acetate, internal standard, and zirconium balls were added into 1 ml samples and homogenized by using zirconium grinding balls with a Retsch mixer mill MM400 for 2 min at 20 Hz at room temperature. Ethyl acetate layer was separated and collected, and the sample was extracted again with 500 μl ethyl acetate. The ethyl acetate layers were combined, evaporated to dryness under a gentle stream of nitrogen and dissolved in 50% methanol.

### Detection of Produced Metabolites

Samples may be separated to biomass and supernatant or the entire biomass and growth medium are subjects to extraction. In the experiments described below, the entire cultivation solution (growth medium and biomass) was extracted. Cannabinoids and their precursors were extracted were extracted as described hereinabove.

All extracellular samples are reconstituted in 50% mobile phase B (0.1% Ammonium hydroxide in Acetonitrile/Methanol (75/25)) before analysis. Intracellular samples are analyzed directly after extraction. Appropriate dilutions of the samples are done when necessary.

The following describes the method developed for analysis of cannabinoids produced by the transgenic fungi of the invention using standard cannabinoid compounds. Cannabinoids and their precursors were analyzed using a quantitative UPLC-MS/MS procedure. Analysis was performed on an Acquity UHPLC system, Waters (Milford, Mass., USA) and Waters Xevo TQ-S MS (Manchester, UK) using an ACQUITY UPLC BEH C18 Column, 1.7 μm, 2.1 mm×100 mm (Waters), kept at 30° C. Injection volume was 2 μl. Separation was performed using gradient elution with 10 mM Ammonium Bicarbonate with 0.1% ammonium hydroxide in water, pH 9.7 (A) and 0.1% ammonium hydroxide in Acetonitrile/Methanol (75/25, v/v) (B) at a flow rate of 0.25 ml/min. Gradient program was as follows: 0 min 90% A, 2.0 min 50% A, 3.0 min 35% A, 3.5 min 90% A, 5.0-7.0 min 5% A and equilibrium time between runs was 2.5 min.

Mass spectrometry was carried out using electrospray ionization in positive polarity (ESI+) (capillary voltage of 1.3 kV) and in negative polarity (ESI−) (capillary voltage 1.5 kV). Desolvation temperature was set to 500° C., and source temperature was set to 150° C. The cone gas flow was 150 l/h (nitrogen), desolvation gas was 1000 l/h (nitrogen), and collision gas was 0.15 ml/min. Analytes were detected using multiple reaction monitoring (MRM) using auto dwell time function. Analytes were quantified by internal standard method. Cannabidiol-D3 (Sigma-Aldrich), (±)-11-nor-9-Carboxy-Δ9-THC-D3 (Sigma-Aldrich) and (±)-Mevalonolactone (Qmx Laboratories) were used as internal standards.

Table 1 summarizes the list of analytes related to cannabinoids, and their precursors, together with the predicted mass of the precursor and product ions, as well as the retention times as determined by the compounds and the methods.

TABLE 1
Precursor and product ions used for MRM, retention times, cone voltage and collision
energy used for the analyzed compounds and the internal standards.

| | | | Precursor | | |
|---|---|---|---|---|---|
| Analyte name | Abbreviation | Polarity | ion | Product ion | RT, min |
| Cannabidiol | CBD | Pos | 315.3 | 193.1 | 6.67 |
| Cannabidiolic acid | CBDA | Neg | 357.3 | 245.1 | 5.73 |
| Cannabigerolic acid | CBGA | Neg | 359.3 | 191.2 | 5.81 |
| Olivetolic acid | OLA | Neg | 225.1 | 189.1 | 2.73 |
| Tetrahydrocannabinol | THC | Pos | 315.1 | 193.1 | 7.27 |
| Δ9-Tetrahydrocannabinolic acid A | THCA | Pos | 359.3 | 219.1 | 6.12 |
| Geranyl pyrophosphate | GPP | Neg | 313.1 | 79.0 | 2.11 |
| Isopentyl pyrophosphate | IPP | Neg | 245.0 | 79.0 | 0.88 |
| Farnesyl pyrophosphate | FPP | Neg | 381.0 | 79.0 | 3.14 |
| Hexanoic acid | HexA | Neg | 161.0 | 57.1 | 0.87 |
| Mevalonic acid | MVA | Neg | 147.1 | 59.1 | 0.96 |
| Mevalonic acid 5-phosphate | MVAP | Neg | 227.1 | 97.0 | 0.84 |
| Mevalonic acid di-phosphate | MVAPP | Neg | 306.9 | 79.0 | 0.80 |
| Cannabidiol-D3 (Istd) | CBD-D3 | Pos | 318.1 | 196.1 | 6.66 |
| (±)-11-nor-9-Carboxy-Δ9-THC-D3 (IStd) | THCA-D3 | Pos | 348.3 | 196.1 | 5.44 |
| Mevalonolactone-d4 (Istd) | MVAL-D4 | Pos | 135.1 | 73.0 | 1.20 |

Linearity, limit of detection (LOD) and limit of quantitation (LOQ) were determined. The calibration curves showed good linearity in the studied range from 0.5 ng/ml to 20,000 ng/ml with correlation coefficient R² greater than 0.99. Limit of detection (LOD) of the method was determined as lowest concentration of the spiked components that could be reliably differentiated from the background level (S/N>3), the limits of quantitation (LOQ) were determined as ratio S/N>10. All results are summarized in Table 2.

TABLE 2
Linearity, limit of detection and limit of quantitation of the method.

| | | Linearity | | | |
|---|---|---|---|---|---|
| | | range, | | LOD | LOQ |
| Analyte | Abbreviation | ng/ml | r\hat{ }2 | ng/ml | ng/ml |
| Cannabidiol | CBD | 0.5-100 | 0.998 | 0.5 | 2.0 |
| Cannabidiolic acid | CBDA | 0.5-100 | 0.998 | 0.5 | 1.0 |
| Cannabigerolic acid | CBGA | 1.0-100 | 0.998 | 0.5 | 1.0 |
| Olivetolic acid | OLA | 0.5-100 | 0.999 | 0.5 | 0.5 |
| Tetrahydrocannabinol | THC | 2.0-100 | 0.999 | 2.0 | 2.0 |
| Δ9- | THCA | 2.0-10000 | 0.999 | 1.0 | 2.0 |
| Tetrahydrocannabinolic | | | | | |
| acid A | | | | | |
| Geranyl pyrophosphate | GPP | 1.0-2000 | 0.999 | 0.5 | 1.0 |
| Isopentyl pyrophosphate | IPP | 50-10000 | 0.999 | 10.0 | 50.0 |
| Farnesyl pyrophosphate | FPP | 2.0-2000 | 0.999 | 1.0 | 2.0 |
| Hexanoic acid | HexA | 200-2000 | 0.999 | nd | nd |
| Mevalonic acid | MVA | 10-20000 | 0.998 | 1.0 | 10.0 |
| Mevalonic acid | MVAP | 5.0-20000 | 0.999 | 5.0 | 20.0 |
| 5-phosphate | | | | | |
| Mevalonic acid | MVAPP | 2.0-20000 | 0.998 | 2.0 | 10.0 |
| diphosphate | | | | | |

### Extraction and Analysis of Hexanoic Acid

The entire cultivation solution (growth medium and biomass) was extracted. The samples were thoroughly vortexed and 1 mL aliquots were taken for the extraction process. The samples were spiked with 10 μL (˜28 μg) of internal standard heptanoic acid (C7) and acidified with 6 M hydrochloric acid (100 μL). The samples were homogenized by using zirconium grinding balls with a Retsch mixer mill MM400 homogenizer at 20 Hz for 5 min. Diethyl ether (500 μL) was used for extraction. The samples were mixed, the phases were allowed to separate and the organic phase was transferred into a GC vial.

A five-point calibration curve was prepared for hexanoic acid (5-50 μg/sample). The samples (1 μL) were run in splitless mode by Agilent GC-MS equipped with a FFAP capillary column (25 m, ID 200 μm, film thickness 0.30 μm; Agilent 19091F-102). The oven temperature program was from 40° C. (1.5 min) to 160° C. at a rate of 10° C./min and then to 240° C. at a rate of 25° C./min. The total run time was 20 min. The MS source and quadrupole temperatures were 230 and 150° C., respectively, and the data were collected from m/z 30 to 600.

### Example 1: Expression Vectors and Construction of Same-General Considerations

DNA sequences are amplified by PCR using appropriate primers and templates, cut by restriction endonucleases from existing constructs or synthesized by DNA synthesis service providers as known in the art.

DNA sequences obtained as above include 5′ regulatory regions (promoters) as are known in the art and described hereinbelow, coding sequences, as described hereinabove, 3′ regulatory regions (terminators) as are known in the art and described hereinbelow, and various targeting sequences.

DNA sequences are assembled to expression cassettes, selection cassettes and further to DNA constructs and/or expression vectors by conventional molecular biological approaches utilizing restriction endonucleases and ligases, Gibson assembly or yeast recombination. Also, the above can be synthesized by DNA synthesis service providers. As known in the art, several different techniques can achieve the same result.

DNA sequences are assembled to expression cassettes joining a 5′ regulatory regions (promoters), a coding sequence and a 3′ regulatory regions (terminators) as described hereinbelow and as are known in the art. Any combination of these three sequences can form a functional expression cassette.

5′ regulatory regions (promoters) known to drive expression of coding sequences in Th. heterothallica at different strength include promoters of Th. heterothallica genes encoding for uncharacterized protein G2QF75 (XP_003664349); polyubiquitin homologue (G2QHM8, XP_003664133); uncharacterized protein (G2QIA5, XP_003664731); beta-glucosidase (G2QD93, XP_003662704); elongation factor 1-alpha (G2Q129, XP_003660173); phosphoglycerate kinase (PGK) (Uniprot G2QLD8), glyceraldehyde 3-phosphate dehydrogenase (GPD) (G2QPQ8), phosphofructokinase (PFK) (G2Q605); or triose phosphate isomerase (TPI) (G2QBRO); actin (ACT) (G2Q7Q5); cbh1 (GenBank AX284115) or β-glucosidase 1 bgl1 (XM_003662656). Exogenous promoters include the promoter of Aspergillus nidulans gpdA. In addition, synthetic promoters which are active in the presence of appropriate exogenous transcription factors are described in Rantasalo et al. (2018 NAR 46(18):e111), which provide very high transcription rates. For example, a synthetic promoter comprising sequences from Th. heterothallica prom1 (G2QF75, XP_003664349) and 8 binding sites of a synthetic transcription factor (sTF) may be used.

The list of coding sequences according to the teachings of the present invention includes C. sativa OLS, C. sativa OAC, C. sativa CBGAS PT1 and PT4, Streptomyces sp. CL190 NphB prenyltransferase, C. sativa CBDAS, C. sativa THCAS, Aspergillus parasiticus strain SU-1 HexA, Aspergillus parasiticus strain SU-1 HexB, C. sativa AAE1, C. sativa AAE3, Th. heterothallica GPPS, various forms of S. cerevisiae ERG20 FPPS, (K197E, F96W-N127W) S. cerevisiae HMG Co-A Reductase (HMG1), two different isoenzymes of Th. heterothallica Fructose-6-phosphate phosphoketolase as described hereinabove and Th. heterothallica acetylphosphatase, as well as any coding sequence that show at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity at the amino acid level, to the polypeptides of the invention as described herein. Any truncations or fusion products as are known in the art and as defined herein are also encompassed in the present invention. The coding sequences are typically codon optimized to be expressed more efficiently in C1.

The list of terminators includes, but are not limited to that of Th. heterothallica genes encoding for uncharacterized protein G2QF75 (XP_003664349); polyubiquitin homologue (G2QHM8, XP_003664133); uncharacterized protein (G2QIA5, XP_003664731); beta-glucosidase (G2QD93, XP_003662704); elongation factor 1-alpha (G2Q129, XP_003660173); chitinase (G2QDD4, XP_003663544) phosphoglycerate kinase (PGK) (Uniprot G2QLD8), glyceraldehyde 3-phosphate dehydrogenase (GPD) (G2QPQ8), phosphofructokinase (PFK) (G2Q605); or triose phosphate isomerase (TPI) (G2QBRO); actin (ACT) (G2Q7Q5); cbh1 (GenBank AX284115) or β-glucosidase 1 bgl1 (XM_003662656). Exogenous terminators include that of Aspergillus nidulans gpdA terminator.

5′ regulatory regions (promoters) are practically defined as a stretch of up to 2000 base pairs preceding the start codon of the coding sequence of the gene they regulate, provided that the preceding region is non-coding.

3′ regulatory regions (terminators) are practically defined as a stretch of up to 300 base pairs downstream from the end codon of the coding sequence of the gene, provided that the subsequent region is non-coding.

DNA sequences are also assembled to selection marker cassettes, which are expression cassettes where the coding sequence codes for a gene that provides a selective advantage when present in a transformed strain. Such advantage can be utilization of a new carbon or nitrogen source, a resistance to a toxic substance etc. More specifically, the selection marker used in the expression cassette of the present invention is amdS, which confers to the transformed fungi the ability to use acetamide as sole nitrogen source, where an Aspergillus nidulans gpdA promoter drives an Aspergillus nidulans amdS gene, and the transcription of which is terminated by its natural Aspergillus nidulans amdS terminator. Hygromycin resistance gene is also used as a selection marker.

DNA constructs used for non-targeted transformation are composed of (a) a suitable vector that allows the maintenance of the DNA construct in a particular host (typically Escherichia coli and/or S. cerevisiae), (b) one or more expression cassettes in any direction and (c) a selection marker cassette in any direction.

DNA constructs used for targeted transformation are composed of (a) a suitable vector that allows the maintenance of the DNA construct in a particular host (typically Escherichia coli and/or S. cerevisiae), (b) zero, one or more expression cassettes in any direction, (c) a selection marker cassette in any direction and (d) sequences that are identical to select stretches of the target genomic DNA (also called as targeting arms). These components are placed so, that the two targeting arms encompass any expression cassettes and the selection marker cassette, so that when homologous recombination happens between the targeting arms and the two identical regions in the genomic DNA, the sequence between the targeting arms of the DNA constructs gets inserted into the chromosome, and replaces the sequence originally present on the chromosome. Using this principle, genes can be knocked out from, or inserted into the genome. By placing a sequence downstream of the selection marker cassette, which is identical to the sequence just upstream of the selection marker cassette, it is possible to recycle the marker as known in the art.

### Example 2: Generation of a Th. Heterothallica Strain Capable of Producing Cannabinoids

Th. heterothallica strain M1889 was used as the host for transformation of cannabinoid pathway genes. M1889 is a ku70-homologue deleted strain. Knocking out the ku70-homologue gene increases the percentage of integration of the transformed DNA through homologous recombination and decrease the percentage of random integration of the transformed DNA.

M1889 was initially transformed simultaneously with two plasmids for the expression of AAE1, AAE3, OLS, OAC, PT4, PT4t, PT1, NphB and CBDAS in various combinations. The genes were introduced to the genome to a suitable locus as detailed in Table 3 hereinbelow. When more than two plasmids are transformed (see Table 3 listing the plasmids according to the transformation order), after the initial transformation of the two plasmids the amdS marker was removed. The resulting marker-deficient isolate was then transformed with the next two plasmids when 4 plasmids are transformed (Table 3). The marker deficient isolate was transformed with one plasmid pCBD0081 when three plasmids are transformed (to create M3808 and M3813).

Th. heterothallica and other filamentous fungi genome is known to comprise genes encoding metabolic enzymes required to produce the precursors for olivetolic acid, including GPP and hexanoyl-CoA. In addition, when using the ant1 locus as the target position, ant1 is disrupted. Loss of the ant1 gene product decreases degradation of short and medium chain fatty acids, including hexanoic acid, and thereby contributing to the increase of availability of cannabinoid precursors.

The plasmids, except for pCBD0081, were designed to have a split marker system, so that a functional marker gene is created only when the two plasmids are joined in a homologous recombination event. Plasmids were digested with MssI, except for pCBD0060, pCBD0068, pCBD0086, pCBD0069, and pCBD0070 that were digested with MssI and SpeI prior to transformation.

The following promoters and terminator were used: prom1 and term 1—the promoter and terminator of a gene coding for a uncharacterized protein G2QF75 [XP_003664349], respectively; prom8 and term8—the promoter and terminator of a gene coding for a polyubiquitin homologue (G2QHM8), respectively [XP_003664133]; prom9 is the promoter of a gene coding for an uncharacterized protein (G2QIA5) [XP_003664731]; bgl8 prom and bgl8 term are the promoter and terminator of a gene coding for a beta-glucosidase (G2QD93) [XP_003662704], and tef1 Aprom is the promoter of the gene coding for elongation factor 1-alpha (G2Q129, XP_003660173), and chi term is the terminator of the gene coding for a chitinase (G2QDD4, XP_003663544). Transformation was performed as described in Example 2 hereinbelow.

Table 3 hereinbelow describes the plasmids used and the composition of the genes introduced.

The selection of transformants was based on acetamidase, encoded by the amdS gene, which enables growth on acetamide plates, resulting in isolation of Th. heterothallica transformants 3-1, M3275, M3277, M3671, M3673, M3593, M3594, M3590 and M3591 (Table 3). The transformants were tested using colony PCR for the presence of the transformed genes and for the absence of the ant1 gene. The oligonucleotide primer pairs used in colony PCR and the size of the expected amplification product are listed in Table 2. The amdS gene in the integrated constructs is flanked by direct repeat sequences, which enabled marker excision upon counter selection on fluoroacetamide (FAA) containing agar plates. amdS resistant strains M3593 and 3-1 were spread onto FAA-plates and the corresponding marker-deficient strains M3713 and M3274, respectively, were isolated.

To increase GPP supply, tHMG1 and a mutated ERG20 gene were transformed into Th. heterothallica. The plasmids were targeted to the bgl8 locus, and a split amdS marker system was used. Strain M3713 was transformed simultaneously with MssI digested plasmids pCBD0114 and pCBD0117, resulting in the isolation of M3806 and with MssI digested plasmids pCBD0115 and pCD0117 resulting in the isolation of strains M3807, and pCBD0081 resulting in the isolation of strains M3808 and M3813. Strain M3274 was transformed simultaneously with MssI digested plasmids pCBD0114 and pCBD0117, resulting in isolation of strains M3837, and with MssI digested plasmids pCBD0115 and pCBD0117 resulting in isolation of strains M3838.

Strain M3714 is transformed simultaneously with different combinations of two MssI digested plasmids, pCBD0114 and pCBD0121 (SEQ ID NO:86), pCBD0114 and pCBD0122 (SEQ ID NO:87), pCBD0115 and pCBD0121, and/or pCBD0115 and pCBD0122.

Strains M3275, M3277, M3274, M3714, M3807, M3837 and M3838 are transformed simultaneously with MssI digested plasmids pCBD0031 (SEQ ID NO:53) and pCBD0032 (SEQ ID NO:53) for hexanoate synthase expression to enhance hexanoic acid biosynthesis in Th. heterothallica. The plasmids are targeted to the cbh1 locus, and a split HygR marker system is used. The selection of transformants is based on hygromycin resistance. To increase GPP supply, tHMG1 and ERG20 derivatives are transformed into hexanoate synthase expressing transformants originating from M3274, M3275, and/or M3714. To this end, M3274-derived hexanoate synthase expressing isolate is transformed simultaneously with MssI digested plasmids pCBD0114 and pCBD0117, and/or with MssI digested plasmids pCBD0115 and pCD0117. The plasmids are targeted to the bgl8 locus, and a split amdS marker system is used. A M32714-derived hexanoate synthase expressing isolate is transformed simultaneously with different combinations of two MssI digested plasmids, pCBD0114 and pCBD0121, pCBD0114 and pCBD0122, pCBD0115 and pCBD0121, and/or pCBD0115 and pCBD0122.

TABLE 3
Transformed strains of Th. heterothallica

| | | Locus of integration:Promoter- | | |
|---|---|---|---|---|
| | Plasmids | GENE-terminator combinations | | |
| | transformed | for the expression of | Genes | |
| Strain | Name/SEQ ID | heterologous genes | deleted | Marker |
| M3275 | pCBD0060/SEQ | ant1Δ:prom1-AAE1-term1, | ku70, | amdS |
| | ID NO: 43; | prom8-OLS-term8, amdS, | ant1 | |
| | pCBD0048/SEQ | prom9-OAC-bgl8 term | | |
| | ID NO: 41 | | | |
| M3277 | pCBD0068/SEQ | ant1Δ:prom1-AAE3-term1, | ku70, | amdS |
| | ID NO: 44; | prom8-OLS-term8, amdS, | ant1 | |
| | pCBD0049/SEQ | prom9-OAC-bgl8 term, bgl8 | | |
| | ID NO: 42 | prom-PT4-bgl8 term | | |
| M1889 | — | — | ku70 | — |
| 3-1 | pCBD0068/SEQ | ant1Δ:prom8-OLS-term8, | ku70, | amdS |
| | ID NO: 44; | prom9-OAC-bgl8 term, amdS, | ant1 | |
| | pCBD0039/SEQ | bgl8 prom-PT4-bgl8 term, | | |
| | ID NO: 40 | prom1-CBDAS-term1 | | |
| M3274 | pCBD0068/SEQ | ant1Δ:prom8-OLS-term8, | ku70, | — |
| | ID NO: 44; | prom9-OCA-bgl8 term, | ant1 | |
| | pCBD0039/SEQ | bgl8 prom-PT4-bgl8 term, | | |
| | ID NO: 40 | prom1-CBDAS-term1 | | |
| M3671 | pCBD0086/SEQ | ant1Δ:prom8-OLS-term8, | ku70, | amdS |
| | ID NO: 48; | prom9-OAC-bgl8 term, amdS, | ant1 | |
| | pCBD0039/SEQ | bgl8 prom-PT4t-bgl8 term, | | |
| | ID NO: 40 | prom1-CBDAS-term1 | | |
| M3673 | pCBD0086/SEQ | ant1Δ:prom1-AAE1-term1, | ku70, | amdS |
| | ID NO: 48; | prom8-OLS-term8, amdS, | ant1 | |
| | pCBD0048/SEQ | prom9-OAC-bgl8 term, bgl8 | | |
| | ID NO: 41 | prom-PT4t-bgl8 term | | |
| M3593 | pCBD0086/SEQ | ant1Δ:prom8-OLS-term8, | ku70, | amdS |
| | ID NO: 48; | prom9-OAC-bgl8 term, amdS, | ant1 | |
| | pCBD0039/SEQ | bgl8 prom-PT4t-bgl8 term, | | |
| | ID NO: 40 | prom1-CBDAS-term1 | | |
| M3594 | pCBD0086/SEQ | ant1Δ:prom1-AAE1-term1, | ku70, | amdS |
| | ID NO: 48; | prom8-OLS-term8, amdS, | ant1 | |
| | pCBD0048/SEQ | prom9-OAC-bgl8 term, bgl8 | | |
| | ID NO: 41 | prom-PT4t-bgl8 term | | |
| M3713 | pCBD0086/SEQ | ant1Δ:prom8-OLS-term8, | ku70, | — |
| | ID NO: 48; | prom9-OAC-bgl8 term, | ant1 | |
| | pCBD0039/SEQ | bgl8 prom-PT4t-bgl8 term, | | |
| | ID NO: 40 | prom1-CBDAS-term1 | | |
| M3714 | pCBD0086/SEQ | ant1Δ:prom1-AAE1-term1, | ku70, | — |
| | ID NO: 48; | prom8-OLS-term8, | ant1 | |
| | pCBD0048/SEQ | prom9-OAC-bgl8 term, bgl8 | | |
| | ID NO: 41 | prom-PT4t-bgl8 term | | |
| M3806 | pCBD0086/SEQ | ant1Δ:prom8-OLS-term8, | ku70, | amdS |
| | ID NO: 48; | prom9-OAC-bgl8 term, bgl8 | ant1, | |
| | pCBD0039/SEQ | prom-PT4t-bgl8 term, | bgl8 | |
| | ID NO: 40; | prom1-CBDAS-term1, | | |
| | pCBD0114/SEQ | bgl8Δ:prom1-tHMG1-term1, | | |
| | ID NO: 49; | prom8-ERG20-K197E-term8, | | |
| | pCBD0117/SEQ | amdS, tef1Aprom-AAE1-term1, | | |
| | ID NO: 51 | | | |
| M3807 | pCBD0086/SEQ | ant1Δ:prom8-OLS-term8, | ku70, | amdS |
| | ID NO: 48; | prom9-OAC-bgl8 term, | ant1, | |
| | pCBD0039/SEQ | bgl8 prom-PT4t-bgl8 term, | bgl8 | |
| | ID NO: 40; | prom1-CBDAS-term1, | | |
| | pCBD0115/SEQ | bgl8Δ:prom1-tHMG1-term1, | | |
| | ID NO: 50; | prom8-ERG20-F96W-N127W-term8, | | |
| | pCBD0117/SEQ | amdS, tef1A prom-AAE1-term1, | | |
| | ID NO: 51 | | | |
| M3812 | pCBD0086/SEQ | ant1Δ:prom8-OLS-term8, | ku70, | amdS |
| | ID NO: 48; | prom9-OAC-bgl8 term, | ant1, | |
| | pCBD0039/SEQ | bgl8 prom-PT4t-bgl8 term, | bgl8 | |
| | ID NO: 40; | prom1-CBDAS-term1, | | |
| | pCBD0115/SEQ | bgl8Δ:prom1-tHMG1-term1, | | |
| | ID NO: 50; | prom8-ERG20-F96W-N127W-term8, | | |
| | pCBD0117/SEQ | amdS, tef1A prom-AAE1-term1 | | |
| | ID NO: 51 | | | |
| M3808 | pCBD0086/SEQ | ant1Δ:prom8-OLS-term8, | ku70, | amdS |
| | ID NO: 48; | prom9-OAC-bgl8 term, | ant1 | |
| | pCBD0039/SEQ | bgl8 prom-PT4t-bgl8 | | |
| | ID NO: 40; | term, prom1-CBDAS-term1, | | |
| | pCBD0081/SEQ | ku70 Δ:prom1-AAE1-term1, amdS | | |
| | ID NO: 47 | | | |
| M3813 | pCBD0086/SEQ | ant1Δ:prom8-OLS-term8, | ku70, | amdS |
| | ID NO: 48; | prom9-OAC-bgl8 term, bgl8 | ant1 | |
| | pCBD0039/SEQ | prom-PT4t-bgl8 term, | | |
| | ID NO: 40; | prom1-CBDAS-term1 | | |
| | pCBD0081/SEQ | ku70 Δ:prom1-AAE1-term1, amdS | | |
| | ID NO: 47 | | | |
| M3837 | pCBD0068/SEQ | ant1Δ:prom8-OLS-term8, | ku70, | amdS |
| | ID NO: 44; | prom9-OAC-bgl8 term, bgl8 | ant1, | |
| | pCBD0039/SEQ | prom-PT4-bgl8 term, | bgl8 | |
| | ID NO: 40; | prom1-CBDAS-term1, | | |
| | pCBD0114/SEQ | bgl8Δ:prom1- tHMG1-term1, | | |
| | ID NO: 49; | prom8-ERG20-K197E-term8, | | |
| | pCBD0117/SEQ | amdS, tef1Aprom-AAE1-term1 | | |
| | ID NO: 51 | | | |
| M3838 | pCBD0068/SEQ | ant1Δ:prom8-OLS-term8, | ku70, | amdS |
| | ID NO: 44; | prom9-OAC-bgl8 term, bgl8 | ant1, | |
| | pCBD0039/SEQ | prom-PT4-bgl8 term, | bgl8 | |
| | ID NO: 40; | prom1-CBDAS-term1, | | |
| | pCBD0115/SEQ | bgl8d:prom1- tHMG1-term1, | | |
| | ID NO: 50; | prom8-ERG20-F96W-N127W-term8, | | |
| | pCBD0117/SEQ | amdS, tef1A prom-AAE1-term1 | | |
| | ID NO: 51 | | | |
| M3590 | pCBD0069/SEQ | ant1Δ:prom8-OLS-term8, | ku70, | amdS |
| | ID NO: 45; | prom9-OAC-bgl8 term, amdS, | ant1 | |
| | pCBD0039/SEQ | bgl8 prom-PT1-bgl8 term, | | |
| | ID NO: 40 | prom1-CBDAS-term1 | | |
| M3591 | pCBD0070/SEQ | ant1Δ:prom8-OLS-term8, | ku70, | amdS |
| | ID NO: 46; | prom9-OAC-bgl8 term, amdS, | ant1 | |
| | pCBD0039/SEQ | bgl8 prom-NphB-bgl8 term, | | |
| | ID NO: 40 | prom1-CBDAS-term1 | | |

### Example 3: Transformation of Th. Heterothallica C1 Cells

Th. heterothallica was cultivated as described hereinabove.

A derivative of Th. heterothallica strains UV18-25, deposit No. VKM F-3631 D, designated herein M1889 was transformed using a conventional PEG mediated protoplast transformation method. Briefly, mycelia were collected by filtration, washed and suspended in protoplasting enzyme mix containing, lysing enzymes from Trichoderma harzianum (Sigma-Aldrich) and optionally Driselase (Sigma-Aldrich). The formation of protoplasts was followed under the microscope. Protoplasts were collected by centrifugation and resuspended in a solution containing sorbitol as the osmotic stabilizer. The transforming DNA optionally linearized by restriction endonucleases and PEG were added into the protoplast suspension and incubated at room temperature for 20-30 min. The protoplasts were again collected by centrifugation and plated onto selection medium.

As a method for selection the amdS selection marker cassette was used, as this allows both positive and negative selection. Briefly, when amdS incorporates to the genome, the expression of the said gene allows the strain to utilize acetamide as a nitrogen source, which is not readily utilized by wildtype C1. The marker can be recycled by culturing the amdS positive cells in the presence of fluoroacetamide. Fluoroacetamide is metabolized by the amdS gene product, which converts fluoroacetamide to fluoroacetate, a metabolic toxin that kills the cells. If the selection marker cassette is flanked by identical sequences, under the selection pressure in a small fraction of the cells the marker cassette is looped out. This way, the amdS selection marker can again be utilized.

The (positive) selection medium for amdS transformants comprises 1.6% Agar noble, 670 mM Sucrose, 7 mM KCl, 11 mM KH₂PO₄, 1% Glucose, 2 mM MgSO₄, 15 mM CsCl, 10 mM acetamide, 1× trace element solution, pH 6.

The (negative) selection medium for amdS marker recycling consists of 2% Agar granulated, 7 mM KCl, 11 mM KH₂PO₄, 100 mM sodium acetate, 0.1% Glucose, 2 mM MgSO₄, 1× trace elements solution, 5 mM Urea, 65 mM Fluoroacetamide, pH 6.

The (positive) selection medium for HygR marker consists of 1.6% Agar noble, 670 mM Sucrose, 35 mM (NH4)₂SO₄, 7 mM KCl, 11 mM KH₂PO₄, 1% Glucose, 10 mM uracil, 2 mM MgSO₄, 15 mM CsCl, 10 mM uridin, 1× trace element solution, 150 μg/ml hygromycin B, pH 6.5.

1000× trace element solution contains 174 mM EDTA, 76 mM ZnSO₄.7H₂O, 178 mM H₃BO₃, 25 mM MnSO₄.H₂O, 18 mM FeSO₄.7H₂O, 7.1 mM CoCl₂.6H₂O, 6.4 mM CuSO₄.5H₂O, 6.2 mM Na₂MoO₄.2H₂O.

As is known to a skilled artisan, other selection markers, or combination of other selection markers can likewise be used to transform and select filamentous fungi.

As known in the art, there are several ways to genotype a strain. For example, the presence of the transforming DNA sequences, the correct integration into a specific locus, and marker excision are verified by colony PCR and/or whole genome sequencing. The oligonucleotides used for detecting the presence of the listed genes in Th. heterothallica transformants using colony PCR are described in Example 4 hereinbelow.

TABLE 4
Oligonucleotides for the detection of
the presence of the listed genes in
Th. heterothallica transformants
using colony PCR

| | | SEQ | PCR |
|---|---|---|---|
| | Oligonucleotides | ID | Product |
| Gene | used | NO | (Bp) |
| OLS | TGCGA | 54 | 670 |
| | CAAGA | | |
| | GCATG | | |
| | ATCCG | | |
| | GGCAC | 55 | |
| | TTCTC | | |
| | GATGT | | |
| | TGTTC | | |
| | G | | |
| OAC | Catac | 56 | 300 |
| | tccaa | | |
| | ctcct | | |
| | gcctg | | |
| | cctta | | |
| | attaa | | |
| | TTAC | | |
| | TTGCG | | |
| | CGGGG | | |
| | TGTAG | | |
| | ctagt | 57 | |
| | ccctc | | |
| | acacc | | |
| | ATGGC | | |
| | CGTCA | | |
| | AGCAC | | |
| | CTC | | |
| AAE1 | ATCAC | 58 | 480 |
| | CTCGG | | |
| | AGGTC | | |
| | GCCGA | | |
| | GACG | | |
| | ATCAC | 59 | |
| | CTCGG | | |
| | AGGTC | | |
| | GCCGA | | |
| | GACG | | |
| AAE3 | ACAAC | 60 | 580 |
| | CTCTC | | |
| | GATGG | | |
| | TCAGC | | |
| | TTCC | | |
| | ACAAC | 61 | |
| | CTCTC | | |
| | GATGG | | |
| | TCAGC | | |
| | TTCC | | |
| CBDAS | TGGTC | 62 | 590 |
| | AAGCT | | |
| | CGTCA | | |
| | ACAAG | | |
| | TGGC | | |
| | GTTGC | 63 | |
| | GGATC | | |
| | CAGTT | | |
| | CAGGT | | |
| | GCTT | | |
| PT4/PT4t | GCTGG | 64 | 400 |
| | AAGCA | | |
| | GTACC | | |
| | CGTTC | | |
| | ACCA | | |
| | TCGCG | 65 | |
| | GGTCT | | |
| | GGAAG | | |
| | ATGAG | | |
| | GCAG | | |
| PT1 | TGCAC | 66 | 1250 |
| | CTTCT | | |
| | CGTTC | | |
| | CAGAC | | |
| | GATGA | 67 | |
| | TCAGG | | |
| | CCGAA | | |
| | GAGGG | | |
| NphB | CCGAG | 68 | 750 |
| | CTCGA | | |
| | CTTCT | | |
| | CCATC | | |
| | TAGTC | 69 | |
| | CTCGA | | |
| | GCGAG | | |
| | TCGAA | | |
| ERG20 | ACCTA | 70 | 390 |
| | CGCCA | | |
| | TCCTG | | |
| | TCCAA | | |
| | AAGCT | 71 | |
| | GTGCT | | |
| | TCTTG | | |
| | AGCGA | | |
| tHMG1 | ACCTC | 72 | 450 |
| | GTACC | | |
| | ACATC | | |
| | CCCAT | | |
| | GAGAC | 73 | |
| | GTCCG | | |
| | ACTTG | | |
| | AGGAC | | |
| hexA | CCTTC | 74 | 560 |
| | AAGGT | | |
| | CTTCC | | |
| | TCAAC | | |
| | CG | | |
| | GTTGT | 75 | |
| | CGTAC | | |
| | ATCTG | | |
| | CTGGA | | |
| | AGTA | | |
| hexB | AGTTG | 76 | 410 |
| | ATGTT | | |
| | GTAGT | | |
| | TGACG | | |
| | ACCT | | |
| | GACCT | 77 | |
| | CCTAC | | |
| | ACCTT | | |
| | CAGCT | | |
| | ACTC | | |
| ant1-3′ | AACCC | 78 | 1100 |
| | TTCCC | | |
| | GACAA | | |
| | CCGCT | | |
| | CCAC | | |
| | GCTGT | 79 | |
| | CTCGG | | |
| | ATCTG | | |
| | GACCA | | |
| | AGTG | | |
| ant1 | TTACC | 80 | 350 |
| | TTACA | | |
| | AGAGC | | |
| | TCGAT | | |
| | CTGC | | |
| | AAGT | 81 | |
| | CACG | | |
| | CTCG | | |
| | ACGTA | | |
| | CAGAT | | |
| | CG | | |
| bgl8 | AACCT | 82 | 1300 |
| | CGAGA | | |
| | CGCTC | | |
| | TTCTA | | |
| | ATCCA | 83 | |
| | CTTGC | | |
| | TTCAC | | |
| | GCT | | |
| bgl8-3’ | GACGC | 84 | 1200 |
| | CCAGC | | |
| | ATTTC | | |
| | ATC | | |
| | AGCGT | 85 | |
| | GACCC | | |
| | ACTCA | | |
| | GGTAA | | |

### Example 5: Th. Heterothallica Suitability for Cannabinoid Production

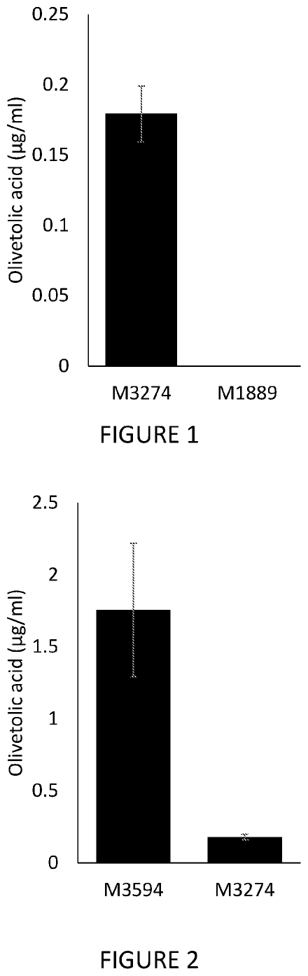

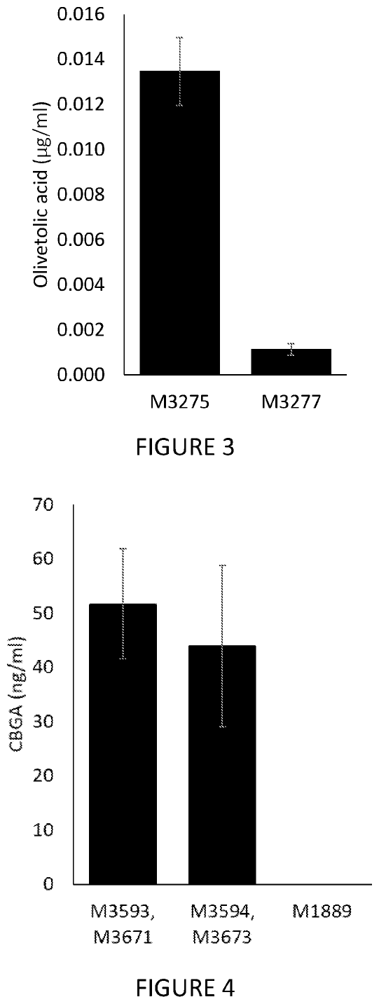

Th. heterothallica strains M3594 (comprising AAE1, OLS, OAC and PT4t), M3274 (lacking heterologous AAE1 and comprising OLS, OAC, and PT4), and the wild type M1889 (Table 3) were grown in complete medium supplemented with 1 mM hexanoic acid (HEX) for 72 h and samples were prepared for metabolite analysis using ethyl acetate extraction as described hereinabove. FIG. 1A shows that strain M3274 produced olivetolic acid (OA) without the presence of a heterologous AAE enzyme. These data support the presence of an endogenous enzyme within Th. heterothallica that is capable of converting hexanoic acid to the precursor hexanoyl-CoA, which was further converted to olivetolic acid by OLS and OAC. The olivetolic acid production may be however increased by further expressing heterologous acyl-activating enzyme (AAE) as in strain M3594 (FIG. 1B). It has been thus further examined which of the potential AAE enzymes may provide for better production of OA. To this end, Th. heterothallica transformed strains M3275 and M3277 (Table 3) expressing C. sativa OLS, OAC, and either AAE1 or AAE3, respectively, were cultivated together with the parent strain M1889, in 24-well plates in 3.5 ml complete medium supplemented with 1 mM hexanoic acid at 35° C. with 800 rpm shaking. Cultures were sampled at 72 h and 1 ml samples containing mycelia and culture medium were prepared using cold methanol extraction. The supernatants were analyzed for the presence of OA. FIG. 1C shows that strain M3275, expressing AAE1, produced more OA than strain M3277, expressing AAE3.

### Example 6: Production of CBGA by Th. Heterothallica

Expression of CBGA was examined in four Th. heterothallica transformed strains: M3593 and its equivalent M3671, expressing C. sativa OLS, OAC, PT4t and CBDAS; and M3594 and it equivalent M3673, expressing C. sativa AAE1, OLS, OAC, PT4t and CBDAS. The strains were cultivated in complete medium supplemented with 1 mM olivetolic acid for 72 h and prepared for analysis using cold methanol extraction. Cannabigerolic acid (CBGA) was produced by the transformants but not the parent strain M1889 (FIG. 2). These data show that PT4t, a mature PT4 protein without a signal peptide was functionally expressed and enabled production of CBGA in Th. heterothallica.

### Example 7: Production of CBDA by Th. Heterothallica

Strains M3837, M3838, M3274, and M1889 are cultivated in complete medium and hexanoic acid is added to final concentration of 0.5 mM at 48 h. Samples are prepared for metabolite analysis using cold methanol extraction at 72 h. Analysis for the presence of CBDA is performed.

T. heterothallica strain M3837 is cultivated along with the parent strain M1889 in complete medium supplemented with 0.5 mM hexanoic acid, at 24 h, at 35° C. with 800 rpm shaking. Hexanoic acid to a final concentration of 1 mM is added at 24 h. Samples are prepared for metabolite analysis using cold methanol extraction at 48 h. The supernatants are analyzed for the presence of CBDA.

### Example 8: Production of Cannabinoids, Cannabinoid Precursors and Derivatives Thereof by Filamentous Fungi

While Th. heterothallica serves in the present invention as an example, other ascomycetous filamentous fungi can be used according to the teachings of the present invention. As described hereinabove, the advantage of using Th. heterothallica for producing cannabinoids and cannabinoid precursors resides, inter alia, in intrinsic biosynthesis pathways providing the initial precursors for olivetolic acid and for CBGA production. To support the hypothesis that ascomycetous filamentous fungi other than Th. heterothallica can be used, the metabolic pathways of several fungi, including Aspergillus nidulans, Penicillium chrysogenum, Rasamsonia emersonii, and Trichoderma reesei were compared. Five alternative genome-scale metabolic models were reconstructed for each species (Castillo et al. unpublished data), and maximum theoretical yields of CBD attainable were simulated using flux balance analysis (FBA) (Varma and Palsson, 1994. Appl Environ Microbiol. 60:3724-31). The maximum theoretical yields of CBD attainable by A. nidulans, P. chrysogenum, R. emersonii, and T. reesei are equal to the maximum theoretical yield attainable by Th. heterothallica (Table 5).

Further, flux variability analysis (FVA) (Mahadevan and Schilling, 2003. Metab Eng. 5:264-76) simulations were performed for identifying the reactions essential for optimally producing CBD. Reactions essential for optimally producing CBD were considered those carrying essentially non-zero fluxes (i.e. range from minimum to maximum flux not including zero) when glucose was converted to CBD at maximum theoretical yield. The set of reactions essential for optimal CBD production was further filtered for reactions heterologous to Th. heterothallica for CBD production, and all transport reactions. When the essential reactions for optimal CBD production of A. nidulans, P. chrysogenum, R. emersonii, and T. reesei were compared to the essential reactions for optimal CBD production by Th. heterothallica, the minimum proportion of shared reactions was at least 85% for all the species (Table 5). Thus, the native metabolism of A. nidulans, P. chrysogenum, R. emersonii, and T. reesei is highly similar for precursor synthesis for CBD production, and those and other equivalent fungi may be used according to the teachings of the invention.

TABLE 5
Maximum theoretical yields of CBD and the minimum proportions of reactions
essential for CBD production shared with Th. heterothallica

| Species | Th. heterothallica | A. nidulans | P. chrysogenum | R. emersonii | T. reesei |
|---|---|---|---|---|---|
| Maximum theoretical | 0.33 | 0.33 | 0.33 | 0.33 | 0.33 |
| yield g CBD/g Glucose | | | | | |
| Minimum proportion of | 1 | 0.93 | 0.90 | 0.85 | 0.88 |
| essential reactions for | | | | | |
| CBD production shared | | | | | |
| with T. heterothallica | | | | | |

### Example 9: Fermentation of the Transformed Strains

For qualification of the generated strains, the strains are fermented in shake flask or in stirred-tank fermenters.

Batch fermentations are conducted in shake flasks in 20 ml batch fermentation medium supplemented with up to about 200 g/l sucrose in 200 ml flat bottomed non-baffled shake flasks overnight at 35° C. with shaking in humidified shakers for 72 to 96 hours.

For 1-liter fed-batch stirred-tank fermentations the seed culture is grown in batch fermentation medium to 100 ml in 1000 ml flat bottomed non-baffled shake flasks as above. The seed culture is then transferred into stirred-tank fermenter containing batch fermentation medium set to pH=6.8. The 1-liter seed culture is further expanded in the fermenter for 24 hours at an aeration rate of 0.6 slpm (standard liter per minute) to increase the biomass at 38° C. pH is maintained at pH 6.8 with addition of 12.5% NH₄OH through a feed line.

After 24 h or as needed feeding is initiated. The feeding rate is set 1-5 g/h. pH is maintained at pH 6.8 with automatic addition of 12.5% NH₄OH. Foaming is controlled as needed. Stirred-tank fermentation is run for 5-7 days. The cultivation is sampled daily or as needed.

Batch fermentation medium contains 10 g/l glucose, 6.26 g/l (NH₄)₂SO₄, 0.47 g/l KH₂PO₄, 0.09 g/l MgSO₄.7H₂O, 1× Trace element solution, 0.03 mg/l biotin and 0.25 mg/l thiamine.

Feed fermentation medium contain 500 g/l glucose, 12.5 g/l (NH₄)₂SO₄, 3.75 g/l KH₂PO₄, 0.75 g/l MgSO₄.7H₂O, 10× Trace element solution, 0.25 mg/l biotin and 2 mg/l thiamine.

It is known in the art that both the media composition and the fermentation process may be modified to optimize the production of cannabinoids, particularly on a commercial scale.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-56. (canceled)

57. A genetically modified ascomycetous filamentous fungus for producing at least one cannabinoid or a precursor thereof selected from the group consisting of cannabigerolic acid, cannabigerolic acid precursor molecule, cannabigerolic acid product, derivatives of same and any combination thereof, wherein the genetically modified ascomycetous filamentous fungus comprises at least one cell comprising at least one of (i) at least one heterologous polynucleotide encoding olivetol synthase (OLS); (ii) at least one heterologous polynucleotide encoding olivetolic acid cyclase (OAC); (iii) at least one heterologous polynucleotide encoding prenyltransferase (PT) having cannabigerolic acid synthase (CBGAS) activity; (iv) at least one heterologous polynucleotide encoding cannabidiolic acid synthase (CBDAS); (v) at least one heterologous polynucleotide encoding tetrahydrocannabinolic acid synthase (THCAS); and a combination thereof.

58. The genetically modified filamentous fungus of claim 57, wherein:
a. the OLS comprises an amino acid sequence having at least 75%, or at least 85%, or at least 90%, or at least 95%, or at least 99%, or 100% identity to the amino acid sequence of C. sativa OLS, wherein the C. sativa OLS comprises the amino acid sequence set forth in SEQ ID NO:1;
b. the OAC comprises an amino acid sequence having at least 75%, or at least 85%, or at least 90%, or at least 95%, or at least 99%, or100% identity to the amino acid sequence of C. sativa OAC, wherein the C. sativa OAC comprises the amino acid sequence set forth in SEQ ID NO:3;
c. the PT comprises an amino acid sequence having at least 75%, or at least 85%, or at least 90%, or at least 95%, or at least 99%, or 100% identity to the amino acid sequence of any one of C. sativa PT4, C. sativa PT1, and Streptomyces sp. CL190 NphB protein, wherein the C. sativa PT4 comprises the amino acid sequence set forth in SEQ ID NO:7, the C. sativa PT1 comprises the amino acid sequence set forth in SEQ ID NO:5, and the Streptomyces sp. CL190 NphB protein comprises the amino acid sequence set forth in SEQ ID NO:9;
d. the CBDAS comprises an amino acid sequence having at least 75%, or at least 85%, or at least 90%, or at least 95%, or at least 99%, or 100% identity to the amino acid sequence of C. sativa CBDAS, wherein the C. sativa CBDAS comprises the amino acid sequence set forth in SEQ ID NO:11;
e. the THCAS comprises an amino acid sequence having at least 75%, or at least 85%, or at least 90%, or at least 95%, or at least 99%, or 100% identity to the amino acid sequence of C. sativa THCAS, wherein the C. sativa THCAS comprises the amino acid sequence set forth in SEQ ID NO:13.

59. The genetically modified ascomycetous filamentous fungus of claim 57, said genetically modified ascomycetous filamentous fungus is further modified to at least one of (i) producing elevated amount of hexanoyl-CoA; (ii) producing elevated amount of geranyl pyrophosphate (GPP); and (iii) overexpressing at least one of said filamentous fungi endogenous enzymes fructose-6-phosphate phosphoketolase and acylphosphatase.

60. The genetically modified ascomycetous filamentous fungus of claim 57, wherein the ascomycetous filamentous fungus is of a genus within Pezizomycotina.

61. The genetically modified ascomycetous filamentous fungus of claim 60 said ascomycetous filamentous fungus is of a genus selected from the group consisting of Thermothelomyces, Myceliophthora, Trichoderma, Aspergillus, Penicillium, Rasamsonia, Chrysosporium, Corynascus, Fusarium, Neurospora, and Talaromyces.

62. The genetically modified ascomycetous filamentous fungus of claim 61, said ascomycetous filamentous fungus is a Thermothelomyces heterothallica or Thermothelomyces thermophila strain comprising rDNA sequence having at least 95%, or at least 96%, or at least 97%, or at least 98%, or at least 99% or 100% identity to the nucleic acid sequence set forth in SEQ ID NO:39.

63. The genetically modified ascomycetous filamentous fungus of claim 62, wherein the at least one heterologous polynucleotide is optimized for expression in Th. heterothallica.

64. The genetically modified ascomycetous filamentous fungus of claim 63, wherein the optimized polynucleotide is selected from the group consisting of a polynucleotide encoding OLS comprising the nucleic acid sequence set forth in SEQ ID NO:2 or an active part thereof; a polynucleotide encoding OAC comprising the nucleic acid sequence set forth in SEQ ID NO:4 or an active part thereof; a polynucleotide encoding C. sativa PT4 comprising the nucleic acid sequence set forth in SEQ ID NO:8 or an active part thereof; a polynucleotide encoding C. sativa PT1 comprising the nucleic acid sequence set forth in SEQ ID NO:6 or an active part thereof; a polynucleotide encoding Streptomyces sp. CL190 NphB protein comprising the nucleic acid sequence set forth in SEQ ID NO:10 or an active part thereof; and a polynucleotide encoding CBDAS comprising the nucleic acid sequence set forth in SEQ ID NO:12 or an active part thereof.

65. The genetically modified ascomycetous filamentous fungus of claim 57, said genetically modified ascomycetous filamentous fungus comprises at least one cell comprising (i) at least one heterologous polynucleotide encoding olivetol synthase (OLS); (ii) at least one heterologous polynucleotide encoding olivetolic acid cyclase (OAC); and (iii) at least one heterologous polynucleotide encoding prenyltransferase (PT) having cannabigerolic acid synthase (CBGAS) activity.

66. The genetically modified ascomycetous filamentous fungus of claim 57, wherein said genetically modified ascomycetous filamentous fungus produces the cannabigerolic acid, at least one cannabigerolic acid precursor and/or at least one cannabigerolic acid product in an increased amount compared to the amount produced in a corresponding unmodified ascomycetous filamentous fungus cultured under similar conditions.

67. The genetically modified ascomycetous filamentous fungus of claim 57, wherein said genetically modified ascomycetous filamentous fungus produces divarinolic acid, products thereof and derivative thereof.

68. A method for producing a fungus capable of producing cannabigerolic acid or cannabigerovarinic acid, at least one cannabigerolic acid or cannabigerovarinic acid precursor, at least one cannabigerolic acid or cannabigerovarinic acid product and/or derivatives of same, the method comprising transforming at least one cell of the fungus with at least one of (i) at least one heterologous polynucleotides encoding olivetol synthase (OLS); (ii) at least one heterologous polynucleotides encoding olivetolic acid cyclase (OAC); (iii) at least one heterologous polynucleotides encoding prenyltransferase (PT) having cannabigerolic acid synthase (CBGAS) activity; (iv) at least one heterologous polynucleotides encoding cannabidiolic acid synthase (CBDAS); and (v) at least one heterologous polynucleotide encoding tetrahydrocannabinolic acid synthase (THCAS) to produce genetically modified fungus capable of producing cannabigerolic acid, at least one cannabigerolic acid precursor, at least one cannabigerolic acid product and/or derivatives of same.

69. The method of claim 68, said method further comprises transforming the at least one cell with at least one polynucleotide selected from the group consisting of a polynucleotide encoding hexanoate synthase; a polynucleotide encoding acyl-activating enzyme; a polynucleotide encoding geranyl-pyrophosphate synthase (GPPS); and a polynucleotide encoding a modified farnesyl pyrophosphate synthase (FPPS) having GPPS activity.

70. The method of claims 68, said method further comprises modulating the expression and/or activity of at least one endogenous enzyme of the fungus fatty acid pathway.

71. The method of claim 68, said method further comprising overexpressing in the at least one cell at least one enzyme selected from the group consisting of fructose-6-phosphate phosphoketolase, acylphosphatase and a combination thereof.

72. The method of claim 68, wherein the genetically modified fungus produces the cannabigerolic acid or cannabigerovarinic acid, the at least one cannabigerolic acid or cannabigerovarinic acid precursor and/or the at least one cannabigerolic acid or cannabigerovarinic acid product in an elevated amount compared to the amount produced by a corresponding unmodified fungus not transformed with the polynucleotides.

73. The method of claim 68, wherein the ascomycetous filamentous fungus is of a genus within Pezizomycotina.

74. The method of claim 73, wherein the ascomycetous filamentous fungus is a Thermothelomyces heterothallica or Thermothelomyces thermophila strain comprising rDNA sequence having at least 95%, or at least 96%, or at least 97%, or at least 98%, or at least 99% or 100% identity to the nucleic acid sequence set forth in SEQ ID NO:39.

75. A method of producing at least one of cannabigerolic acid or cannabigerovarinic acid, at least one cannabidiolic acid or cannabigerovarinic acid precursor, at least one cannabidiolic acid or cannabigerovarinic acid product and/or derivatives of same, the method comprising culturing the genetically modified fungus of claim 57 in a suitable medium; and recovering the produced at least one of cannabigerolic acid or cannabigerovarinic acid, at least one cannabigerolic acid or cannabigerovarinic acid precursor at least one cannabigerolic acid or cannabigerovarinic acid product and/or derivatives of same.

76. A cannabigerolic acid or cannabigerovarinic acid, cannabigerolic acid or cannabigerovarinic acid precursor, cannabidiolic acid or cannabigerovarinic acid product and/or a derivative of same produced by the method of claim 75.


SEQUENCE LISTING (91 sequences)

SEQ ID NO: 1 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 2 - DNA, 1158 bp, Artificial Sequence
  Synthetic Polynucleotide
atgaaccacctgcgcgccgagggcccggcctcggtcctcgccatcggcaccgccaaccccgagaacatcctcctgcaggacgagttcccggactactacttccgcgtcaccaagtccgagcacatgacccagctgaaggagaagttccgcaagatctgcgacaagagcatgatccgcaagcgcaactgcttcctcaacgaggagcacctgaagcagaacccgcgcctcgtcgagcacgagatgcagaccctggacgcccgccaggacatgctggtcgtcgaggtccccaagctgggcaaggacgcctgcgccaaggccatcaaggagtggggccagccgaagtcgaagatcacccacctgatcttcacctcggcctccaccaccgacatgccgggcgccgactaccactgcgccaagctgctgggcctctccccctcggtcaagcgcgtcatgatgtaccagctgggctgctacggtggcggcaccgtcctccgcatcgccaaggacatcgccgagaacaacaagggcgcccgcgtcctggccgtctgctgcgacatcatggcctgcctgttccgcggcccctccgagtcggacctggagctcctggtcggccaggccatcttcggcgacggcgccgccgccgtcatcgtcggcgccgagcccgacgagtcggtcggcgagcgcccgatcttcgagctggtcagcaccggccagaccatcctgcccaactcggagggcaccatcggcggccacatccgcgaggccggcctcatcttcgacctgcacaaggacgtcccgatgctgatctcgaacaacatcgagaagtgcctcatcgaggccttcacccccatcggcatcagcgactggaactcgatcttctggatcacccaccctggcggcaaggccatcctcgacaaggtcgaggagaagctccacctgaagtccgacaagttcgtcgactcccgccacgtcctgtcggagcacggcaacatgagctcgtccaccgtcctcttcgtcatggacgagctccgcaagcgctcgctggaggaaggcaagtcgaccaccggcgacggcttcgagtggggcgtcctgttcggcttcggcccgggcctcaccgtcgagcgcgtcgtcgtccgcagcgtcccgatcaagtactaa

SEQ ID NO: 3 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 4 - DNA, 306 bp, Artificial Sequence
  Synthetic Polynucleotide
atggccgtcaagcacctcatcgtcctgaagttcaaggacgagatcaccgaggcccagaaggaagagttcttcaagacctacgtcaacctcgtcaacatcatccccgccatgaaggacgtctactggggcaaggacgtcacccagaagaacaaggaagagggctacacccacatcgtcgaggtcaccttcgagagcgtcgagacgatccaggactacatcatccacccggcccacgtcggcttcggcgacgtctaccgctcgttctgggagaagctcctgatcttcgactacaccccgcgcaagtaa

SEQ ID NO: 5 - protein, 395 aa, Cannabis sativa
MGLSSVCTFSFQTNYHTLLNPHNNNPKTSLLCYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 6 - DNA, 1188 bp, Artificial Sequence
  Synthetic Polynucleotide
atgggcctcagctcggtctgcaccttctcgttccagaccaactaccacaccctcctgaacccccacaacaacaacccgaagacctccctcctgtgctaccgccaccccaagaccccgatcaagtacagctacaacaacttcccctcgaagcactgctcgaccaagtccttccacctccagaacaagtgctccgagagcctgtcgatcgccaagaactcgatccgcgccgccaccaccaaccagaccgagcctcccgagtccgacaaccacagcgtcgccaccaagatcctcaacttcggcaaggcctgctggaagctgcagcgcccgtacaccatcatcgccttcacctcctgcgcctgcggcctcttcggcaaggagctcctgcacaacaccaacctcatctcctggagcctgatgttcaaggccttcttcttcctcgtcgccatcctgtgcatcgcctcgttcaccacgaccatcaaccagatctacgacctccacatcgaccgcatcaacaagccggacctccccctggcctccggcgagatctccgtcaacaccgcctggatcatgtccatcatcgtcgccctcttcggcctgatcatcaccatcaagatgaagggcggccccctctacatcttcggctactgcttcggcatcttcggtggcatcgtctacagcgtcccgcccttccgctggaagcagaacccgtcgaccgccttcctcctgaacttcctcgcccacatcatcaccaacttcaccttctactacgcctcccgcgccgccctgggcctgcccttcgagctgcgcccgagcttcaccttcctcctggccttcatgaagagcatgggctccgccctggccctgatcaaggacgccagcgacgtcgagggcgacaccaagttcggcatcagcaccctcgcctcgaagtacggctcccgcaacctcaccctgttctgctccggcatcgtcctgctcagctacgtcgccgccatcctggccggcatcatctggccccaggccttcaactcgaacgtcatgctcctgtcccacgccatcctcgccttctggctcatcctgcagacccgcgacttcgccctgaccaactacgaccccgaggccggccgcaggttctacgagttcatgtggaagctctactacgccgagtacctggtctacgtcttcatctaa

SEQ ID NO: 7 - protein, 398 aa, Cannabis sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 8 - DNA, 1197 bp, Artificial Sequence
  Synthetic Polynucleotide
atgggcctcagcctcgtctgcaccttcagcttccagaccaactaccacacgctcctcaacccgcacaacaagaaccccaagaacagcctcctgtcctaccagcaccccaagaccccgatcatcaagagctcgtacgacaacttcccctccaagtactgcctgaccaagaacttccacctcctgggcctcaacagccacaaccgcatctcctcgcagagccgctccatccgcgccggctcggaccagatcgagggctcgccccaccacgagagcgacaactcgatcgccaccaagatcctgaacttcggccacacctgctggaagctccagcgcccgtacgtcgtcaagggcatgatctccatcgcctgcggcctgttcggccgcgagctcttcaacaaccgccacctgttctcgtggggcctcatgtggaaggccttcttcgccctggtcccgatcctctccttcaacttcttcgccgccatcatgaaccagatctacgacgtcgacatcgaccgcatcaacaagccggacctgccgctcgtctcgggcgagatgtccatcgagacggcctggatcctcagcatcatcgtcgccctgaccggcctcatcgtcaccatcaagctgaagtcggccccgctcttcgtcttcatctacatcttcggcatcttcgccggcttcgcctacagcgtcccgcccatccgctggaagcagtacccgttcaccaacttcctgatcaccatctcgtcccacgtcggcctcgccttcacctcctactcggccaccaccagcgccctgggcctccccttcgtctggcgcccggccttctcgttcatcatcgccttcatgaccgtcatgggcatgaccatcgccttcgccaaggacatctcggacatcgagggcgacgccaagtacggcgtctccaccgtcgccaccaagctgggcgcccgcaacatgaccttcgtcgtcagcggcgtcctcctgctcaactacctcgtctcgatctccatcggcatcatctggccccaggtcttcaagtccaacatcatgatcctcagccacgccatcctggccttctgcctcatcttccagacccgcgagctggccctcgccaactacgcctccgccccgagccgccagttcttcgagttcatctggctcctctactacgccgagtacttcgtctacgtcttcatctga

SEQ ID NO: 9 - protein, 307 aa, Streptomyces sp. (strain CL190) [sequence omitted]

SEQ ID NO: 10 - DNA, 924 bp, Artificial Sequence
  Synthetic Polynucleotide
atgtccgaggccgccgacgtcgagcgcgtctacgccgccatggaggaagccgccggcctcctcggcgtcgcctgcgcccgcgacaagatctaccccctcctgagcaccttccaggacaccctggtcgagggcggctccgtcgtcgtcttcagcatggcctccggccgccactccaccgagctcgacttctccatctcggtccccaccagccacggcgacccgtacgccaccgtcgtcgagaagggcctgttcccggccaccggccaccccgtcgacgacctcctggccgacacccagaagcacctccccgtcagcatgttcgccatcgacggcgaggtcaccggcggcttcaagaagacctacgccttcttcccgaccgacaacatgcccggcgtcgccgagctctccgccatcccctccatgcctcccgccgtcgccgagaacgccgagctgttcgcccgctacggcctcgacaaggtccagatgacctcgatggactacaagaagcgccaggtcaacctctacttctcggagctgtcggcccagaccctggaggccgagtcggtcctggccctggtccgcgagctgggcctgcacgtccccaacgagctcggcctgaagttctgcaagcgctcgttctccgtctacccgaccctgaactgggagacgggcaagatcgaccgcctctgcttcgccgtcatctccaacgacccgaccctggtccccagctccgacgagggcgacatcgagaagttccacaactacgccaccaaggccccctacgcctacgtcggcgagaagcgcaccctggtctacggcctcaccctgagcccgaaggaagagtactacaagctcggcgcctactaccacatcaccgacgtccagcgcggcctcctgaaggccttcgactcgctcgaggactaa

SEQ ID NO: 11 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 12 - DNA, 1635 bp, Artificial Sequence
  Synthetic Polynucleotide
atgaagtgctcaacattctccttttggtttgtttgcaagataatatttttctttttctcattcaatatccaaacttccattgctaacccccgcgagaacttcctcaagtgcttctcgcagtacatcccgaacaacgccaccaacctgaagctcgtctacacccagaacaaccccctgtacatgtccgtcctcaacagcaccatccacaacctgcgcttcaccagcgacaccacccccaagccgctcgtcatcgtcaccccgtcgcacgtctcccacatccagggcaccatcctgtgctcgaagaaggtcggcctccagatccgcacccgcagcggcggccacgactcggagggcatgagctacatctcgcaggtccccttcgtcatcgtcgacctgcgcaacatgcgctccatcaagatcgacgtccacagccagaccgcctgggtcgaggccggcgccaccctcggcgaggtctactactgggtcaacgagaagaacgagaacctgtccctggccgccggctactgccccaccgtctgcgctggcggccacttcggtggcggcggctacggccccctgatgcgcaactacggcctcgccgccgacaacatcatcgacgcccacctggtcaacgtccacggcaaggtcctcgaccgcaagtccatgggcgaggacctgttctgggccctcaggggcggcggcgccgagagcttcggcatcatcgtcgcctggaagatccgcctggtcgccgtccccaagtcgaccatgttctccgtcaagaagatcatggagatccacgagctggtcaagctcgtcaacaagtggcagaacatcgcctacaagtacgacaaggacctcctgctcatgacccacttcatcacccgcaacatcaccgacaaccagggcaagaacaagaccgccatccacacctacttctcgtccgtcttcctcggcggcgtcgactccctggtcgacctcatgaacaagtccttcccggagctgggcatcaagaagaccgactgccgccagctcagctggatcgacaccatcatcttctactcgggcgtcgtcaactacgacaccgacaacttcaacaaggagatcctgctggaccgctccgccggccagaacggcgccttcaagatcaagctggactacgtcaagaagcccatcccggagtccgtcttcgtccagatcctggagaagctctacgaggaagacatcggcgccggcatgtacgccctctacccgtacggtggcatcatggacgagatctccgagtcggccatccccttcccccaccgcgccggcatcctgtacgagctctggtacatctgctcctgggagaagcaggaagacaacgagaagcacctgaactggatccgcaacatctacaacttcatgaccccctacgtcagcaagaacccgcgcctggcctacctcaactaccgcgacctcgacatcggcatcaacgaccccaagaacccgaacaactacacccaggcccgcatctggggcgagaagtacttcggcaagaacttcgaccgcctggtcaaggtcaagaccctcgtcgaccccaacaacttcttccgcaacgagcagagcatcccgcccctcccgcgccaccgccactaa

SEQ ID NO: 13 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 14 - DNA, 1638 bp, Cannabis sativa
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 15 - protein, 1671 aa, Aspergillus parasiticus
MVIQGKRLAASSIQLLASSLDAKKLCYEYDERQAPGVTQITEEAPTEQPPLSTPPSLPQTPNISPISASKIVIDDVALSRVQIVQALVARKLKTAIAQLPTSKSIKELSGGRSSLQNELVGDIHNEFSSIPDAPEQILLRDFGDANPTVQLGKTSSAAVAKLISSKMPSDFNANAIRAHLANKWGLGPLRQTAVLLYAIASEPPSRLASSSAAEEYWDNVSSMYAESCGITLRPRQDTMNEDAMASSAIDPAVVAEFSKGHRRLGVQQFQALAEYLQIDLSGSQASQSDALVAELQQKVDLWTAEMTPEFLAGISPMLDVKKSRRYGSWWNMARQDVLAFYRRPSYSEFVDDALAFKVFLNRLCNRADEALLNMVRSLSCDAYFKQGSLPGYHAASRLLEQAITSTVADCPKARLILPAVGPHTTITKDGTIEYAEAPRQGVSGPTAYIQSLRQGASFIGLKSADVDTQSNLTDALLDAMCLALHNGISFVGKTFLVTGAGQGSIGAGVVRLLLEGGARVLVTTSREPATTSRYFQQMYDNHGAKFSELRVVPCNLASAQDCEGLIRHVYDPRGLNWDLDAILPFAAASDYSTEMHDIRGQSELGHRLMLVNVFRVLGHIVHCKRDAGVDCHPTQVLLPLSPNHGIFGGDGMYPESKLALESLFHRIRSESWSDQLSICGVRIGWTRSTGLMTAHDIIAETVEEHGIRTFSVAEMALNIAMLLTPDFVAHCEDGPLDADFTGSLGTLGSIPGFLAQLHQKVQLAAEVIRAVQAEDEHERFLSPGTKPTLQAPVAPMHPRSSLRVGYPRLPDYEQEIRPLSPRLERLQDPANAVVVVGYSELGPWGSARLRWEIESQGQWTSAGYVELAWLMNLIRHVNDESYVGWVDTQTGKPVRDGEIQALYGDHIDNHTGIRPIQSTSYNPERMEVLQEVAVEEDLPEFEVSQLTADAMRLRHGANVSIRPSGNPDACHVKLKRGAVILVPKTVPFVWGSCAGELPKGWTPAKYGIPENLIHQVDPVTLYTICCVAEAFYSAGITHPLEVFRHIHLSELGNFIGSSMGGPTKTRQLYRDVYFDHEIPSDVLQDTYLNTPAAWVNMLLLGCTGPIKTPVGACATGVESIDSGYESIMAGKTKMCLVGGYDDLQEEASYGFAQLKATVNVEEEIACGRQPSEMSRPMAESRAGFVEAHGCGVQLLCRGDIALQMGLPIYAVIASSAMAADKIGSSVPAPGQGILSFSRERARSSMISVTSRPSSRSSTSSEVSDKSSLTSITSISNPAPRAQRARSTTDMAPLRAALATWGLTIDDLDVASLHGTSTRGNDLNEPEVIETQMRHLGRTPGRPLWAICQKSVTGHPKAPAAAWMLNGCLQVLDSGLVPGNRNLDTLDEALRSASHLCFPTRTVQLREVKAFLLTSFGFGQKGGQVVGVAPKYFFATLPRPEVEGYYRKVRVRTEAGDRAYAAAVMSQAVVKIQTQNPYDEPDAPRIFLDPLARISQDPSTGQYRFRSDATPALDDDALPPPGEPTELVKGISSAWIEEKVRPHMSPGGTVGVDLVPLASFDAYKNAIFVERNYTVRERDWAEKSADVRAAYASRWCAKEAVFKCLQTHSQGAGAAMKEIEIEHGGNGAPKVKLRGAAQTAARQRGLEGVQLSISYGDDAVIAVALGLMSGAS

SEQ ID NO: 16 - DNA, 5016 bp, Artificial Sequence
  Synthetic Polynucleotide
atggtcatccagggcaagcggctggccgccagctccatccagctcctggcctccagcctggacgccaagaagctctgctacgagtacgacgagcgccaggccccgggcgtcacccagatcaccgaggaagccccgaccgagcagcccccgctctccaccccgcccagcctgccgcagacccccaacatcagccccatcagcgcctcgaagatcgtcatcgacgacgtcgccctctcccgcgtccagatcgtccaggccctggtcgcccgcaagctcaagaccgccatcgcccagctcccgacctccaagagcatcaaggagctgtcgggcggccgctcctccctgcagaacgagctcgtcggcgacatccacaacgagttctcgtccatccccgacgccccggagcagatcctcctccgcgacttcggcgacgccaaccccaccgtccagctgggcaagacctcctcggccgccgtcgccaagctgatctcgtccaagatgcccagcgacttcaacgccaacgccatccgcgcccacctcgccaacaagtggggcctcggccccctgcgccagaccgccgtcctcctgtacgccatcgcctccgagcctccctcgcgcctggccagctcctcggccgccgaggagtactgggacaacgtcagctcgatgtacgccgagtcgtgcggcatcaccctgcgcccccgccaggacaccatgaacgaggacgccatggcctcgtcggccatcgaccccgccgtcgtcgccgagttctccaagggccaccgcaggctgggcgtccagcagttccaggccctggccgagtacctccagatcgacctgtccggctcccaggccagccagtccgacgccctcgtcgccgagctgcagcagaaggtcgacctgtggaccgccgagatgaccccggagttcctggccggcatctcgccgatgctggacgtcaagaagtcgcgcaggtacggctcctggtggaacatggcccgccaggacgtcctggccttctaccgcaggccctcctacagcgagttcgtcgacgacgccctggccttcaaggtcttcctcaaccgcctgtgcaaccgcgccgacgaggccctcctgaacatggtccgctcgctctcctgcgacgcctacttcaagcagggctccctgccgggctaccacgccgccagccgcctcctggagcaggccatcaccagcaccgtcgccgactgcccgaaggcccgcctcatcctgccggccgtcggcccgcacaccaccatcaccaaggacggcaccatcgagtacgccgaggcccccaggcagggcgtctcgggccccaccgcctacatccagtcgctgcgccagggcgccagcttcatcggcctgaagtcggccgacgtcgacacccagtccaacctcaccgacgccctcctggacgccatgtgcctcgccctgcacaacggcatctccttcgtcggcaagaccttcctggtcaccggcgccggccagggcagcatcggcgccggcgtcgtccgcctcctgctcgagggcggcgcccgcgtcctcgtcaccacctcccgcgagcccgccaccaccagccgctacttccagcagatgtacgacaaccacggcgccaagttctcggagctgcgcgtcgtcccctgcaacctcgcctccgcccaggactgcgagggcctcatccgccacgtctacgacccgcgcggcctcaactgggacctcgacgccatcctgcccttcgccgccgcctccgactactccaccgagatgcacgacatccgcggccagtccgagctgggccaccgcctcatgctggtcaacgtcttccgcgtcctgggccacatcgtccactgcaagcgcgacgccggcgtcgactgccacccgacccaggtcctgctccccctctcgccgaaccacggcatcttcggcggcgacggcatgtacccggagtccaagctcgccctggagagcctcttccaccgcatccgcagcgagtcgtggtccgaccagctgtccatctgcggcgtccgcatcggctggacccgcagcaccggcctcatgaccgcccacgacatcatcgccgagacggtcgaggagcacggcatccgcaccttcagcgtcgccgagatggccctcaacatcgccatgctgctcacccccgacttcgtcgcccactgcgaggacggccccctggacgccgacttcaccggctcgctcggcaccctgggctcgatcccgggcttcctcgcccagctgcaccagaaggtccagctggccgccgaggtcatccgcgccgtccaggccgaggacgagcacgagcgcttcctctccccgggcaccaagcccaccctgcaggcccccgtcgcccccatgcacccccgctcgtccctccgcgtcggctacccccgcctgccggactacgagcaggagatccgccccctcagcccgcgcctggagcgcctgcaggaccccgccaacgccgtcgtcgtcgtcggctactccgagctgggcccctggggctcggcccgcctgcgctgggagatcgagagccagggccagtggacctccgccggctacgtcgagctggcctggctgatgaacctcatccgccacgtcaacgacgagagctacgtcggctgggtcgacacccagaccggcaagccggtccgcgacggcgagatccaggccctctacggcgaccacatcgacaaccacaccggcatccgccccatccagagcacctcgtacaacccggagcgcatggaggtcctccaggaagtcgccgtcgaggaagacctgccggagttcgaggtcagccagctcaccgccgacgccatgcgcctgcgccacggcgccaacgtcagcatccgcccctccggcaaccccgacgcctgccacgtcaagctcaagaggggcgccgtcatcctggtccccaagaccgtcccgttcgtctggggcagctgcgccggcgagctgccgaagggctggacccccgccaagtacggcatccccgagaacctcatccaccaggtcgacccggtcaccctgtacaccatctgctgcgtcgccgaggccttctactccgccggcatcacccaccccctggaggtcttccgccacatccacctgtccgagctcggcaacttcatcggctcgtccatgggcggccccaccaagacccgccagctgtaccgcgacgtctacttcgaccacgagatcccctcggacgtcctccaggacacctacctcaacacccccgccgcctgggtcaacatgctgctcctgggctgcaccggccccatcaagacccccgtcggcgcctgcgccaccggcgtcgagagcatcgactccggctacgagagcatcatggccggcaagaccaagatgtgcctggtcggcggctacgacgacctgcaggaagaggcctcgtacggcttcgcccagctcaaggccaccgtcaacgtcgaggaagagatcgcctgcggccgccagccctcggagatgagccgcccgatggccgagagccgcgccggcttcgtcgaggcccacggctgcggcgtccagctcctgtgccgcggcgacatcgccctgcagatgggcctccccatctacgccgtcatcgcctcctcggccatggccgccgacaagatcggctcctcggtccccgccccgggccagggcatcctctccttcagccgcgagcgcgcccgcagctcgatgatctccgtcacctcccgcccgtcctcgcgctcctccaccagctccgaggtcagcgacaagtccagcctgacctcgatcacctcgatctccaaccccgcccccagggcccagcgcgcccgctcgaccaccgacatggccccgctccgcgccgccctcgccacctggggcctgaccatcgacgacctggacgtcgccagcctgcacggcacctccacccgcggcaacgacctcaacgagcccgaggtcatcgagacgcagatgcgccacctgggccgcaccccgggcaggcccctgtgggccatctgccagaagtccgtcaccggccaccccaaggcccccgccgccgcctggatgctcaacggctgcctgcaggtcctggactcgggcctggtccccggcaaccgcaacctggacaccctggacgaggccctgcgctcggcctcccacctgtgcttccccacccgcaccgtccagctccgcgaggtcaaggccttcctcctgacctccttcggcttcggccagaagggcggccaggtcgtcggcgtcgcccccaagtacttcttcgccaccctgcccaggcccgaggtcgagggctactaccgcaaggtccgcgtccgcaccgaggccggcgaccgcgcctacgccgccgccgtcatgagccaggccgtcgtcaagatccagacccagaacccctacgacgagccggacgccccgcgcatcttcctggaccccctggcccgcatctcccaggaccccagcaccggccagtaccgcttccgctcggacgccacccccgccctcgacgacgacgccctgcccccgcccggcgagccgaccgagctcgtcaagggcatctcgtcggcctggatcgaggagaaggtccgcccccacatgtcccctggcggcaccgtcggcgtcgacctggtccccctggcctccttcgacgcctacaagaacgccatcttcgtcgagcgcaactacaccgtccgcgagcgcgactgggccgagaagtccgccgacgtccgcgccgcctacgcctcccgctggtgcgccaaggaagccgtcttcaagtgcctgcagacgcacagccagggcgccggcgccgccatgaaggagatcgagatcgagcacggtggcaacggcgccccgaaggtcaagctgaggggcgccgcccagaccgccgcccgccagcgcggcctcgagggcgtccagctgtccatcagctacggcgacgacgccgtcatcgccgtcgccctgggcctgatgtcgggcgcctcgtaa

SEQ ID NO: 17 - protein, 1888 aa, Artificial Sequence
  Synthetic Polynucleotide
MGSVSREHESIPIQAAQRGAARICAAFGGQGSNNLDVLKGLLELYKRYGPDLDELLDVASNTLSQLASSPAAIDVHEPWGFDLRQWLTTPEVAPSKEILALPPRSFPLNTLLSLALYCATCRELELDPGQFRSLLHSSTGHSQGILAAVAITQAESWPTFYDACRTVLQISFWIGLEAYLFTPSSAASDAMIQDCIEHGEGLLSSMLSVSGLSRSQVERVIEHVNKGLGECNRWVHLALVNSHEKFVLAGPPQSLWAVCLHVRRIRADNDLDQSRILFRNRKPIVDILFLPISAPFHTPYLDGVQDRVIEALSSASLALHSIKIPLYHTGTGSNLQELQPHQLIPTLIRAITVDQLDWPLVCRGLNATHVLDFGPGQTCSLIQELTQGTGVSVIQLTTQSGPKPVGGHLAAVNWEAEFGLRLHANVHGAAKLHNRMTTLLGKPPVMVAGMTPTTVRWDFVAAVAQAGYHVELAGGGYHAERQFEAEIRRLATAIPADHGITCNLLYAKPTTFSWQISVIKDLVRQGVPVEGITIGAGIPSPEVVQECVQSIGLKHISFKPGSFEAIHQVIQIARTHPNFLIGLQWTAGRGGGHHSWEDFHGPILATYAQIRSCPNILLVVGSGFGGGPDTFPYLTGQWAQAFGYPCMPFDGVLLGSRMMVAREAHTSAQAKRLIIDAQGVGDADWHKSFDEPTGGVVTVNSEFGQPIHVLATRGVMLWKELDNRVFSIKDTSKRLEYLRNHRQEIVSRLNADFARPWFAVDGHGQNVELEDMTYLEVLRRLCDLTYVSHQKRWVDPSYRILLLDFVHLLRERFQCAIDNPGEYPLDIIVRVEESLKDKAYRTLYPEDVSLLMHLFSRRDIKPVPFIPRLDERFETWFKKDSLWQSEDVEAVIGQDVQRIFIIQGPMAVQYSISDDESVKDILHNICNHYVEALQADSRETSIGDVHSITQKPLSAFPGLKVTTNRVQGLYKFEKVGAVPEMDVLFEHIVGLSKSWARTCLMSKSVFRDGSRLHNPIRAALQLQRGDTIEVLLTADSEIRKIRLISPTGDGGSTSKVVLEIVSNDGQRVFATLAPNIPLSPEPSVVFCFKVDQKPNEWTLEEDASGRAERIKALYMSLWNLGFPNKASVLGLNSQFTGEELMITTDKIRDFERVLRQTSPLQLQSWNPQGCVPIDYCVVIAWSALTKPLMVSSLKCDLLDLLHSAISFHYAPSVKPLRVGDIVKTSSRILAVSVRPRGTMLTVSADIQRQGQHVVTVKSDFFLGGPVLACETPFELTEEPEMVVHVDSEVRRAILHSRKWLMREDRALDLLGRQLLFRLKSEKLFRPDGQLALLQVTGSVFSYSPDGSTTAFGRVYFESESCTGNVVMDFLHRYGAPRAQLLELQHPGWTGTSTVAVRGPRRSQSYARVSLDHNPIHVCPAFARYAGLSGPIVHGMETSAMMRRIAEWAIGDADRSRFRSWHITLQAPVHPNDPLRVELQHKAMEDGEMVLKVQAFNERTEERVAEADAHVEQETTAYVFCGQGSQRQGMGMDLYVNCPEAKALWARADKHLWEKYGFSILHIVQNNPPALTVHFGSQRGRRIRANYLRMMGQPPIDGRHPPILKGLTRNSTSYTFSYSQGLLMSTQFAQPALALMEMAQFEWLKAQGVVQKGARFAGHSLGEYAALGACASFLSFEDLISLIFYRGLKMQNALPRDANGHTDYGMLAADPSRIGKGFEEASLKCLVHIIQQETGWFVEVVNYNINSQQYVCAGHFRALWMLGKICDDLSCHPQPETVEGQELRAMVWKHVPTVEQVPREDRMERGRATIPLPGIDIPYHSTMLRGEIEPYREYLSERIKVGDVKPCELVGRWIPNVVGQPFSVDKSYVQLVHGITGSPRLHSLLQQMA

SEQ ID NO: 18 - DNA, 5667 bp, Artificial Sequence
  Synthetic Polynucleotide
atgggctccgtcagccgcgagcacgagtccatccccatccaggccgcccagaggggcgccgcccgcatctgcgccgccttcggtggccagggcagcaacaacctggacgtcctcaagggcctcctggagctgtacaagcgctacggcccggacctggacgagctgctggacgtcgccagcaacaccctctcccagctggccagctcccccgccgccatcgacgtccacgagccctggggcttcgacctgcgccagtggctcaccacccccgaggtcgcccccagcaaggagatcctggccctcccgccccgcagcttccccctcaacaccctcctgtccctcgccctctactgcgccacctgccgcgagctcgagctggacccgggccagttccgctcgctcctccactccagcaccggccactcccagggcatcctggccgccgtcgccatcacccaggccgagtcctggcccaccttctacgacgcctgccgcaccgtcctccagatcagcttctggatcggcctggaggcctacctgttcaccccctcctcggccgcctccgacgccatgatccaggactgcatcgagcacggcgagggcctcctgagctcgatgctgagcgtcagcggcctctcgcgctcgcaggtcgagcgcgtcatcgagcacgtcaacaagggcctgggcgagtgcaaccgctgggtccacctggccctcgtcaactcccacgagaagttcgtcctggccggcccgccccagtcgctctgggccgtctgcctccacgtccgccgcatccgcgccgacaacgacctggaccagtcccgcatcctcttccgcaaccgcaagccgatcgtcgacatcctgttcctccccatctcggcccccttccacaccccctacctggacggcgtccaggaccgcgtcatcgaggccctctcctcggcctccctggccctccacagcatcaagatccccctctaccacaccggcaccggctccaacctgcaggagctccagccccaccagctgatcccgaccctcatccgcgccatcaccgtcgaccagctggactggcccctggtctgccgcggcctgaacgccacccacgtcctggacttcggcccgggccagacctgcagcctgatccaggagctcacccagggcaccggcgtctccgtcatccagctgaccacccagtcgggccccaagcccgtcggcggccacctggccgccgtcaactgggaggccgagttcggcctgcgcctccacgccaacgtccacggcgccgccaagctccacaaccgcatgaccaccctgctcggcaagcctcccgtcatggtcgccggcatgacccccaccaccgtccgctgggacttcgtcgccgccgtcgcccaggccggctaccacgtcgagctcgctggcggcggctaccacgccgagcgccagttcgaggccgagatccgccgcctggccaccgccatccccgccgaccacggcatcacctgcaacctcctgtacgccaagcccaccaccttctcctggcagatcagcgtcatcaaggacctggtccgccagggcgtcccggtcgagggcatcaccatcggcgccggcatcccctcccccgaggtcgtccaggagtgcgtccagagcatcggcctcaagcacatctcgttcaagccgggctccttcgaggccatccaccaggtcatccagatcgcccgcacccaccccaacttcctgatcggcctccagtggaccgccggcaggggcggcggccaccacagctgggaggacttccacggccccatcctggccacctacgcccagatccgctcctgccccaacatcctcctggtcgtcggctcgggcttcggtggcggcccggacaccttcccctacctgaccggccagtgggcccaggccttcggctacccctgcatgcccttcgacggcgtcctcctgggctcgcgcatgatggtcgcccgcgaggcccacacctcggcccaggccaagcgcctcatcatcgacgcccagggcgtcggcgacgccgactggcacaagagcttcgacgagcccaccggcggcgtcgtcaccgtcaactcggagttcggccagcccatccacgtcctggccacccgcggcgtcatgctgtggaaggagctcgacaaccgcgtcttcagcatcaaggacacctcgaagcgcctggagtacctccgcaaccaccgccaggagatcgtctcccgcctgaacgccgacttcgccaggccctggttcgccgtcgacggccacggccagaacgtcgagctcgaggacatgacctacctggaggtcctccgcaggctgtgcgacctcacctacgtcagccaccagaagcgctgggtcgacccctcgtaccgcatcctcctgctcgacttcgtccacctgctccgcgagcgcttccagtgcgccatcgacaacccgggcgagtaccccctggacatcatcgtccgcgtcgaggagagcctgaaggacaaggcctaccgcaccctctacccggaggacgtctccctgctcatgcacctcttcagccgcagggacatcaagccggtccccttcatcccgcgcctggacgagcgcttcgagacgtggttcaagaaggactccctgtggcagtcggaggacgtcgaggccgtcatcggccaggacgtccagcgcatcttcatcatccagggcccgatggccgtccagtactccatcagcgacgacgagtcggtcaaggacatcctgcacaacatctgcaaccactacgtcgaggccctgcaggccgacagccgcgagacgtccatcggcgacgtccactcgatcacccagaagccgctgtcggccttccccggcctcaaggtcaccaccaaccgcgtccagggcctctacaagttcgagaaggtcggcgccgtcccggagatggacgtcctgttcgagcacatcgtcggcctctccaagtcctgggcccgcacctgcctgatgtcgaagtccgtcttccgcgacggctcgcgcctccacaacccgatccgcgccgccctgcagctccagcgcggcgacaccatcgaggtcctgctcaccgccgactccgagatccgcaagatccgcctgatctcccccaccggcgacggtggctccaccagcaaggtcgtcctcgagatcgtctcgaacgacggccagcgcgtcttcgccaccctcgcccccaacatccccctctccccggagccctcggtcgtcttctgcttcaaggtcgaccagaagccgaacgagtggaccctcgaggaagacgcctccggccgcgccgagcgcatcaaggccctctacatgtccctgtggaacctcggcttccccaacaaggcctcggtcctgggcctcaactcccagttcaccggcgaggagctgatgatcaccaccgacaagatccgcgacttcgagcgcgtcctgcgccagaccagcccgctgcagctgcagtcctggaacccccagggctgcgtccccatcgactactgcgtcgtcatcgcctggagcgccctgaccaagcccctcatggtcagctccctgaagtgcgacctgctcgacctgctccactccgccatcagcttccactacgccccctccgtcaagcccctccgcgtcggcgacatcgtcaagaccagctcgcgcatcctggccgtcagcgtccgcccccgcggcaccatgctcaccgtctcggccgacatccagcgccagggccagcacgtcgtcaccgtcaagtcggacttcttcctgggcggcccggtcctggcctgcgagacgccgttcgagctcaccgaggagcccgagatggtcgtccacgtcgactccgaggtccgcagggccatcctgcactcgcgcaagtggctcatgcgcgaggaccgggccctggacctgctgggccgccagctgctcttccgcctgaagtccgagaagctcttccgccccgacggccagctggccctgctccaggtcaccggcagcgtcttctcgtactcgccggacggctccaccaccgccttcggccgcgtctacttcgagagcgagtcgtgcaccggcaacgtcgtcatggacttcctgcaccgctacggcgcccccagggcccagctgctcgagctccagcaccccggctggaccggcaccagcaccgtcgccgtccgcggcccccgccgctcccagagctacgcccgcgtctcgctggaccacaacccgatccacgtctgccccgccttcgcccgctacgccggcctcagcggccccatcgtccacggcatggagacgtcggccatgatgcgcaggatcgccgagtgggccatcggcgacgccgaccgctcgcgcttccgctcgtggcacatcaccctgcaggccccggtccaccccaacgacccgctgcgcgtcgagctccagcacaaggccatggaggacggcgagatggtcctcaaggtccaggccttcaacgagcgcaccgaggagcgcgtcgccgaggccgacgcccacgtcgagcaggagacgaccgcctacgtcttctgcggccagggcagccagcgccagggcatgggcatggacctctacgtcaactgcccggaggccaaggccctgtgggcccgcgccgacaagcacctctgggagaagtacggcttctccatcctgcacatcgtccagaacaacccgcccgccctcaccgtccacttcggcagccagcgcggccgccgcatccgcgccaactacctccgcatgatgggccagcctcccatcgacggccgccacccgcccatcctgaagggcctcacccgcaactcgacctcctacaccttcagctactcgcagggcctgctcatgtcgacccagttcgcccagccggccctggccctcatggagatggcccagttcgagtggctcaaggcccagggcgtcgtccagaagggcgcccgcttcgccggccacagcctgggcgagtacgccgccctcggcgcctgcgcctcgttcctctccttcgaggacctgatctcgctcatcttctaccgcggcctgaagatgcagaacgccctcccccgcgacgccaacggccacaccgactacggcatgctggccgccgacccctcccgcatcggcaagggcttcgaggaagccagcctgaagtgcctcgtccacatcatccagcaggagacgggctggttcgtcgaggtcgtcaactacaacatcaactcgcagcagtacgtctgcgccggccacttccgcgccctgtggatgctcggcaagatctgcgacgacctgtcctgccacccccagcccgagacggtcgagggccaggagctccgcgccatggtctggaagcacgtccccaccgtcgagcaggtcccgcgcgaggaccgcatggagcgcggccgcgccaccatccccctccccggcatcgacatcccgtaccactcgaccatgctccgcggcgagatcgagccctaccgcgagtacctctccgagcgcatcaaggtcggcgacgtcaagccctgcgagctggtcggccgctggatccccaacgtcgtcggccagccgttcagcgtcgacaagtcgtacgtccagctggtccacggcatcacgggcagcccccgcctccacagcctgctccagcagatggcctaa

SEQ ID NO: 19 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 20 - DNA, 2163 bp, Artificial Sequence
  Synthetic Polynucleotide
atgggcaagaactacaagagcctggactcggtcgtcgcctccgacttcatcgccctgggcatcacctcggaggtcgccgagacgctccacggccgcctggccgagatcgtctgcaactacggcgccgccaccccgcagacctggatcaacatcgccaaccacatcctctcgcccgacctgccgttctccctccaccagatgctgttctacggctgctacaaggacttcggccccgcccctcccgcctggatcccggaccccgagaaggtcaagtccaccaacctgggcgccctcctggagaagcgcggcaaggagttcctcggcgtcaagtacaaggaccccatcagctcgttcagccacttccaggagttctcggtccgcaacccggaggtctactggcgcaccgtcctgatggacgagatgaagatcagcttctcgaaggaccccgagtgcatcctccgcagggacgacatcaacaaccctggcggctcggagtggctccctggcggctacctgaactcggccaagaactgcctgaacgtcaactccaacaagaagctcaacgacaccatgatcgtctggcgcgacgagggcaacgacgacctccccctgaacaagctcaccctcgaccagctgcgcaagcgcgtctggctggtcggctacgccctggaggagatgggcctcgagaagggctgcgccatcgccatcgacatgccgatgcacgtcgacgccgtcgtcatctacctcgccatcgtcctggccggctacgtcgtcgtcagcatcgccgactcgttctcggccccggagatctccacccgcctccgcctgagcaaggccaaggccatcttcacccaggaccacatcatccgcggcaagaagcgcatcccgctgtactcgcgcgtcgtcgaggccaagtcccccatggccatcgtcatcccctgctccggctcgaacatcggcgccgagctgcgcgacggcgacatcagctgggactacttcctcgagcgcgccaaggagttcaagaactgcgagttcaccgcccgcgagcagccggtcgacgcctacaccaacatcctcttctcctcgggcaccaccggcgagcccaaggccatcccctggacccaggccaccccgctgaaggccgccgccgacggctggtcgcacctcgacatccgcaagggcgacgtcatcgtctggcccaccaacctgggctggatgatgggcccctggctggtctacgcctccctcctgaacggcgcctccatcgccctgtacaacggcagccccctcgtctcgggcttcgccaagttcgtccaggacgccaaggtcaccatgctgggcgtcgtcccctccatcgtccgctcctggaagagcaccaactgcgtctccggctacgactggagcaccatccgctgcttctcctcgtcgggcgaggccagcaacgtcgacgagtacctctggctgatgggccgcgccaactacaagcccgtcatcgagatgtgcggtggcaccgagatcggcggcgccttctccgccggctccttcctgcaggcccagtccctctcgtccttcagctcgcagtgcatgggctgcaccctctacatcctggacaagaacggctaccccatgccgaagaacaagccgggcatcggcgagctggccctgggcccggtcatgttcggcgcctcgaagaccctcctgaacggcaaccaccacgacgtctacttcaagggcatgcccaccctcaacggcgaggtcctccgcaggcacggcgacatcttcgagctcacctccaacggctactaccacgcccacggccgcgccgacgacaccatgaacatcggcggcatcaagatctccagcatcgagatcgagcgcgtctgcaacgaggtcgacgaccgcgtcttcgagacgaccgccatcggcgtcccgcccctcggcggcggccccgagcagctggtcatcttcttcgtcctcaaggacagcaacgacaccaccatcgacctcaaccagctccgcctgtcgttcaacctcggcctgcagaagaagctcaacccgctgttcaaggtcacccgcgtcgtccccctctcctccctgccccgcaccgccaccaacaagatcatgcgcagggtcctccgccagcagttcagccacttcgagtaa

SEQ ID NO: 21 - protein, 543 aa, Cannabis sativa
MEKSGYGRDGIYRSLRPPLHLPNNNNLSMVSFLFRNSSSYPQKPALIDSETNQILSFSHFKSTVIKVSHGFLNLGIKKNDVVLIYAPNSIHFPVCFLGIIASGAIATTSNPLYTVSELSKQVKDSNPKLIITVPQLLEKVKGFNLPTILIGPDSEQESSSDKVMTFNDLVNLGGSSGSEFPIVDDFKQSDTAALLYSSGTTGMSKGVVLTHKNFIASSLMVTMEQDLVGEMDNVFLCFLPMFHVFGLAIITYAQLQRGNTVISMARFDLEKMLKDVEKYKVTHLWVVPPVILALSKNSMVKKFNLSSIKYIGSGAAPLGKDLMEECSKVVPYGIVAQGYGMTETCGIVSMEDIRGGKRNSGSAGMLASGVEAQIVSVDTLKPLPPNQLGEIWVKGPNMMQGYFNNPQATKLTIDKKGWVHTGDLGYFDEDGHLYVVDRIKELIKYKGFQVAPAELEGLLVSHPEILDAVVIPFPDAEAGEVPVAYVVRSPNSSLTENDVKKFIAGQVASFKRLRKVTFINSVPKSASGKILRRELIQKVRSNM

SEQ ID NO: 22 - DNA, 1632 bp, Artificial Sequence
  Synthetic Polynucleotide
atggagaagtccggctacggccgcgacggcatctaccgcagcctccgcccgcccctccacctgcccaacaacaacaacctctcgatggtcagcttcctcttccgcaacagctcgtcctacccccagaagccggccctcatcgactcggagacgaaccagatcctgtcgttctcccacttcaagtcgaccgtcatcaaggtcagccacggcttcctcaacctgggcatcaagaagaacgacgtcgtcctgatctacgcccccaactccatccacttcccggtctgcttcctcggcatcatcgcctcgggcgccatcgccaccacctccaaccccctctacaccgtcagcgagctgtcgaagcaggtcaaggactccaaccccaagctgatcatcaccgtcccgcagctcctggagaaggtcaagggcttcaacctccccaccatcctgatcggccccgactcggagcaggagagctcctccgacaaggtcatgaccttcaacgacctggtcaacctgggcggcagctccggctcggagttccccatcgtcgacgacttcaagcagtccgacaccgccgccctcctgtactcgtcgggcaccaccggcatgagcaagggcgtcgtcctcacccacaagaacttcatcgcctcgtccctcatggtcaccatggagcaggacctggtcggcgagatggacaacgtcttcctctgcttcctgccgatgttccacgtcttcggcctcgccatcatcacctacgcccagctgcagcgcggcaacaccgtcatctcgatggcccgcttcgacctcgagaagatgctgaaggacgtcgagaagtacaaggtcacccacctctgggtcgtcccgcccgtcatcctcgccctgtccaagaacagcatggtcaagaagttcaacctcagctcgatcaagtacatcggctccggcgccgccccgctcggcaaggacctgatggaggagtgctccaaggtcgtcccctacggcatcgtcgcccagggctacggcatgaccgagacgtgcggcatcgtcagcatggaggacatcaggggcggcaagcgcaacagcggctccgccggcatgctcgcctcgggcgtcgaggcccagatcgtctcggtcgacaccctgaagcccctgcccccgaaccagctcggcgagatctgggtcaagggccccaacatgatgcagggctacttcaacaacccgcaggccaccaagctcaccatcgacaagaagggctgggtccacaccggcgacctcggctacttcgacgaggacggccacctgtacgtcgtcgaccgcatcaaggagctcatcaagtacaagggcttccaggtcgccccggccgagctggagggcctcctggtcagccacccggagatcctcgacgccgtcgtcatccccttccccgacgccgaggccggcgaggtccccgtcgcctacgtcgtccgctcgccgaactccagcctgaccgagaacgacgtcaagaagttcatcgccggccaggtcgcctccttcaagcgcctccgcaaggtcaccttcatcaacagcgtcccgaagtcggcctcgggcaagatcctccgcagggagctgatccagaaggtccgctcgaacatgtaa

SEQ ID NO: 23 - protein, 347 aa, Thermothelomyces heterothallica
MAKQTNLKEFEAVFPKLEKVLLEHAEQYKLPKQVVDWYKKSLEVNTLGGKCNRGMSVPDSASLLLGRPLTEDEYFRAATLGWMTELLQAFFLVSDDIMDGSITRRGKPCWYRHEGVGMIAINDAFMLESAIYTLLKKFFRSHPRYVDLLELFHEVTFQTEIGQLCDLLTAPEDVVNLDNFSMEKYRFIVIYKTAYYSFYLPVALALYLLDIATPGNLKQAEDILIPLGEYFQVQDDYLDNFGLPEHIGKIGTDIQDNKCSWLVNQALAIVTPEQRRVLEENYGRKDKTKEAAVKKLYDELKLEQRYKEYEEKAVGDIRGLIDKIDESQGLRKGVFEAFLAKIYKRSK

SEQ ID NO: 24 - DNA, 1044 bp, Thermothelomyces heterothallica
atggcgaagcaaacaaacctcaaggagttcgaggccgtcttccctaagctggagaaggttctcctcgaacatgccgagcagtacaagctcccgaagcaggtcgtcgactggtacaagaaatccctcgaggtcaacacccttggcggaaagtgcaaccgcggcatgtcggtgccggactcggcgtcgctgctcttggggcgccccctaaccgaggacgagtacttccgggccgcgacgctgggttggatgacggagctgctgcaggccttcttcctggtgtctgacgacatcatggacggcagcatcacgcggcgcggcaagccctgctggtaccgccacgagggcgtcggcatgatcgccatcaacgacgccttcatgctcgagtcggccatctacacgctcctcaagaagttcttccgctcccacccgcgctacgtcgacctgctcgagctgttccacgaggttaccttccagaccgagattggccagctgtgcgacctgctcaccgcccccgaggacgtcgtcaatctcgacaacttcagcatggagaagtaccgcttcatcgtcatctacaagacggcctactacagtttctacctgcccgtcgccctggcgctgtacctgctcgacatcgccacccccgggaacctcaagcaggccgaggatatcctcatcccgctgggcgagtacttccaggtgcaggacgactacctcgacaacttcggcctgcccgagcacatcggcaagatcggcaccgacatccaggacaacaagtgctcgtggctggtcaaccaggcgctggccatcgtgacccccgagcagcgccgcgtgctcgaggagaactacggccgcaaggacaagaccaaggaggccgccgtcaagaagctgtacgacgagctcaagctggagcagcggtacaaggagtacgaggagaaggctgtcggcgacatccgcggcttgatcgacaagatcgacgagtcccagggcctgagaaagggcgtcttcgaggccttcctggccaagatttacaagcgcagcaaataa

SEQ ID NO: 25 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFETAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 26 - DNA, 1059 bp, Artificial Sequence
  Synthetic Polynucleotide
atggccagcgagaaggagatccgccgcgagaggttcctcaacgtgttccccaagctcgtcgaggagctgaacgccagcctcctcgcctacggcatgcccaaggaggcctgcgactggtacgcccacagcctcaactacaacacgcccggcggcaagctcaaccgcggcctcagcgtcgtcgacacctacgccatcctcagcaacaagaccgtcgagcagctcggccaggaggagtacgagaaggtcgcgatcctcggctggtgcatcgagctgctgcaggcctacttcctcgtcgccgacgacatgatggacaagagcatcacccgcaggggccagccgtgctggtacaaggtccccgaggtcggcgagatcgccatcaacgacgccttcatgctcgaggccgccatctacaagctcctcaagagccacttccgcaacgagaagtactacatcgacatcaccgagctgttccacgaggtcaccttccagaccgagctgggccagctgatggacctcatcacggcgcccgaggacaaggtggacctcagcaagttcagcctcaagaagcacagcttcatcgtcacgttcgaaaccgcctactacagcttctacctgccggtcgcgctcgccatgtacgtcgccggcatcaccgacgagaaggacctcaagcaggcccgcgacgtgctcatcccgctcggcgagtacttccagatccaggacgactacctcgactgcttcggcacgcccgagcagatcggcaagatcggcaccgacatccaggacaacaagtgcagctgggtcatcaacaaggccctcgagctggcctccgccgagcagcgcaagaccctggacgagaactacggcaagaaggacagcgtcgccgaggccaagtgcaagaagatcttcaacgacctcaagatcgagcagctgtaccacgagtacgaggagtcgatcgccaaggacctgaaggccaagatcagccaggtcgacgagtcgcgcggcttcaaggccgacgtcctgaccgccttcctcaacaaggtctacaagcgcagcaagtga

SEQ ID NO: 27 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYWLVADDMMDKSITRRGQPCWYKVPEVGEIAIWDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 28 - DNA, 1059 bp, Artificial Sequence
  Synthetic Polynucleotide
atggcctccgagaaggagatccgccgcgagcgcttcctgaacgtcttccccaagctggtcgaggagctcaacgcctcgctcctggcctacggcatgccgaaggaagcctgcgactggtacgcccactccctcaactacaacacccctggcggcaagctgaaccgcggcctctccgtcgtcgacacctacgccatcctgtccaacaagaccgtcgagcagctcggccaggaagagtacgagaaggtcgccatcctgggctggtgcatcgagctcctgcaggcctactggctcgtcgccgacgacatgatggacaagtcgatcacccgcaggggccagccctgctggtacaaggtccccgaggtcggcgagatcgccatctgggacgccttcatgctggaggccgccatctacaagctcctgaagagccacttccgcaacgagaagtactacatcgacatcaccgagctcttccacgaggtcaccttccagaccgagctcggccagctgatggacctcatcaccgccccggaggacaaggtcgacctgagcaagttctcgctcaagaagcacagcttcatcgtcaccttcaagaccgcctactactcgttctacctgccggtcgccctggccatgtacgtcgccggcatcaccgacgagaaggacctgaagcaggcccgcgacgtcctgatccccctcggcgagtacttccagatccaggacgactacctcgactgcttcggcacccccgagcagatcggcaagatcggcaccgacatccaggacaacaagtgctcctgggtcatcaacaaggccctggagctggcctcggccgagcagcgcaagaccctcgacgagaactacggcaagaaggacagcgtcgccgaggccaagtgcaagaagatcttcaacgacctgaagatcgagcagctctaccacgagtacgaggagtcgatcgccaaggacctcaaggccaagatctcccaggtcgacgagtcgcgcggcttcaaggccgacgtcctgaccgccttcctcaacaaggtctacaagcgcagcaagtaa

SEQ ID NO: 29 - protein, 525 aa, Saccharomyces cerevisiae
MDQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 30 - DNA, 1578 bp, Artificial Sequence
  Synthetic Polynucleotide
atggaccagctcgtcaagaccgaggtcaccaagaagtccttcaccgcccccgtccagaaggccagcacccccgtcctcaccaacaagaccgtcatctccggcagcaaggtcaagagcctgtcctccgcccagtcgtcctcctcgggccccagctcctcctcggaggaagacgacagccgcgacatcgagtcgctcgacaagaagatccgccccctggaggagctggaggccctcctgtcctccggcaacaccaagcagctcaagaacaaggaagtcgccgccctggtcatccacggcaagctcccgctgtacgccctcgagaagaagctgggcgacaccacccgcgccgtcgccgtccgcaggaaggccctcagcatcctggccgaggcccccgtcctggcctccgaccgcctgccctacaagaactacgactacgaccgcgtcttcggcgcctgctgcgagaacgtcatcggctacatgccgctccccgtcggcgtcatcggccccctggtcatcgacggcacctcgtaccacatccccatggccaccaccgagggctgcctggtcgcctcggccatgcgcggctgcaaggccatcaacgctggcggcggcgccaccaccgtcctgaccaaggacggcatgacccgcggccccgtcgtccgcttccccaccctcaagcgctccggcgcctgcaagatctggctggactccgaggaaggccagaacgccatcaagaaggccttcaactccacctcgcgcttcgcccgcctccagcacatccagacctgcctggccggcgacctcctgttcatgcgcttccgcaccaccaccggcgacgccatgggcatgaacatgatctcgaagggcgtcgagtactccctcaagcagatggtcgaggagtacggctgggaggacatggaggtcgtcagcgtctcgggcaactactgcaccgacaagaagcccgccgccatcaactggatcgagggccgcggcaagtcggtcgtcgccgaggccaccatcccgggcgacgtcgtccgcaaggtcctcaagtcggacgtctccgccctcgtcgagctgaacatcgccaagaacctggtcggctcggccatggccggctcggtcggcggcttcaacgcccacgccgccaacctcgtcaccgccgtcttcctcgccctgggccaggacccggcccagaacgtcgagagctcgaactgcatcaccctcatgaaggaagtcgacggcgacctgcgcatctccgtcagcatgccgtcgatcgaggtcggcaccatcggcggcggcaccgtcctcgagccccagggcgccatgctggacctcctgggcgtccgcggcccccacgccaccgcccccggcaccaacgcccgccagctcgcccgcatcgtcgcctgcgccgtcctggccggcgagctctccctgtgcgccgccctcgccgccggccacctggtccagagccacatgacccacaaccgcaagccggccgagcccaccaagcccaacaacctcgacgccaccgacatcaaccgcctgaaggacggcagcgtcacctgcatcaagtcgtaa

SEQ ID NO: 31 - protein, 815 aa, Thermothelomyces heterothallica
MPRKQIPIANPPPLPSHLPDSVLELAVQPEKKPLQDDVRQSLRSFQRAAEYITAAMIFLRDNVLLDSELKMENIKPRLLGHWGTCPGLILVWSHLNLLIRNHDLDMIYVIGPGHGAPGALAALWLEGSLEKFYPGQYDRNAEGLRNLITRFSVPGGFPSHINAQTPGSIHEGGELGYALAVSFGAVMDNPDLIVTCIVGDGEAETGPTATAWHAIKYLDPAESGAVIPILHANGFKISERTIFGCMDDKEIVCLFSGYGYQVRIVEDLEDIDDELQNALEWAVAEIKKIQQAARSGKPIEKPRWPMIVLRTPKGWTGPKEVEGNLIEGSFHAHQVPLPKANSDPTQLKALDNWLSSYKIGELLKDGKPTETVLALLPRKDGKKLGQLKATYAPFIGLKAVDWQPFGVEKGSEESCMKVTGKFLDKVFQENPKTIRLFSPDELESNKLDAVLDHSQRNFQWDQYSRANGGRVIEVLSEHNCQGFMQGYTLTGRTAIFPSYESFLGIVHTMMVQYSKFVKIGCEVKWRGDLPSINYIETSTWTRQEHNGFSHQNPSFIGAVLNIKPRAARVYLPPDANCFLSTVHHCLQSRNKTNLIIGSKQPTAVYLSPEEAAEHCRRGASIWSFASSPPDPSRQAQEDEPDVVLVGIGVEVTFETVKAAELLRALCPRLRVRVVNVTDLMILAPEARHPHALSRDAFVDLFTADRPVLFNYHGYATELQGLLFCHPGTARMSIAGYREEGSTTTPFDMMLVNRVSRFDLARKALQVAAERNAEVREKAEALIKDMDARVDEVKRFIVQHGKDPDDIYKPPKFDHN

SEQ ID NO: 32 - DNA, 2448 bp, Thermothelomyces heterothallica
atgcctagaaaacagattccgatcgcgaatccccctcctctgccgtctcacctcccagacagcgtgctggagctggcggtgcagcccgagaagaagccgctgcaagatgacgtccgacagtcattgagaagcttccagcgggcggcagagtacatcacagcagcgatgatattcctccgggacaatgtccttcttgactccgaactgaagatggaaaatatcaagccccgtctcttaggccactggggaacatgtccgggtctcatcttagtgtggtcccacctcaacctgctcatccgcaaccacgacctcgacatgatctatgttattggtccagggcacggtgcgccgggcgccttggctgcgttgtggctcgagggctctctggagaagttctaccctggccagtacgacaggaatgcggaagggttgcgaaacttgatcaccagattctccgttcccgggggctttccaagccacatcaacgcccagactcccggatccatccacgagggcggcgagctggggtatgcgctggccgtatccttcggtgcagtcatggataatccagacctcatcgtcacttgcatcgttggcgacggggaggccgagacggggccgactgctacggcctggcacgccatcaagtaccttgacccggccgaatcgggggccgtcattcccattctccatgccaacgggttcaagatcagcgaacggaccatattcggctgtatggacgacaaagagattgtttgcttgttcagcggctacggctatcaagtccgcatagtggaagatctggaagacatcgacgacgagctgcagaacgcccttgagtgggccgtagctgagatcaagaagattcagcaagcggcgcggtcggggaagccgatagagaagccacgatggccaatgatagtgttaaggacgccgaagggttggactgggcccaaggaggttgagggcaacctcatcgaaggatcctttcatgctcatcaggttcctttgcccaaggccaacagcgacccaacacagctcaaagcgctcgacaactggctgtcgagctacaaaatcggcgagctcctcaaggacgggaagccgaccgagaccgttctcgcccttttgcctcgcaaggacgggaagaaactcggtcagctcaaggcgacatacgcccctttcattggccttaaggccgtcgactggcaaccgtttggcgtcgagaagggcagcgaggagagttgcatgaaggtcaccggcaagttcctcgacaaggtcttccaggagaaccccaagacgatcagactcttctcgcctgacgagctggagagcaacaagctcgacgctgtgctcgaccattcgcaacgcaacttccagtgggaccagtactcgcgagccaacggtgggcgcgtgattgaggtgctctcggagcacaattgccagggcttcatgcaagggtacacactgacgggccggaccgctatcttcccgagctatgagtcattcctgggcatcgtgcacaccatgatggttcagtactccaagttcgtcaaaatcggatgtgaggtcaagtggcgcggcgacctgccgtcgatcaactatatcgagacgagcacgtggactcggcaagagcacaatgggttctcgcaccagaacccgtcgtttatcggtgcagtcttgaacatcaagcctagggcagctagggtctatctccccccggacgcgaactgcttcctcagcaccgtccaccactgcctccagtcacgcaataagaccaatctcatcatcggttccaagcagcccacggcagtgtacctgtcacccgaggaagcggccgagcattgccgccgcggcgcctccatttggtctttcgcctcgtcccctcccgacccgtcccggcaggcgcaggaagatgagccggacgttgtgctggtcggcatcggcgtcgaggtgacctttgagacggtcaaggcggccgagctactgcgagccctgtgcccgcggctgcgcgtccgcgtcgtcaacgtgacggacctgatgatcctggcgcccgaggctcggcacccgcacgcgctgagccgcgatgcctttgtcgacctcttcacggccgaccgccccgtgctgttcaactaccacggctacgccaccgagctacagggcctgctgttttgtcaccccggcaccgcacgcatgagcatcgccgggtaccgcgaggagggcagcacgacaacgccgtttgacatgatgctcgtcaatagggtgagccggttcgacctggcaaggaaggcgctgcaggtcgctgcagagaggaacgcggaggtgagggaaaaggctgaagcgctaattaaggatatggatgcacgggtggatgaggtcaaacggttcattgtccaacatgggaaggatcccgacgacatctacaagccacccaagtttgatcacaactaa

SEQ ID NO: 33 - protein, 823 aa, Thermothelomyces heterothallica
MGDANELESISTFGSARSTIKGAPLSEEEVKKYNDFFKASLYLSLGMIYLRHNPLLKEPLKKEHLKARLLGHFGSAPGQIFTYMHFNRLINKYDLDALFISGPGHGAPAVLSQAYLEGTYSEVYPDKSEDEEGLQKFFKHFSFPGGIGSHATPETPGSLHEGGELGYSISHAFGAVFDNPNLIALTMVGDGEAETGPLATAWHSNKFLNPITDGAVLPVLHLNGYKINNPTILARISHKELENLFLGYGYQPYFVEGDEVDSMHQAMAATLEHCVLEIRKYQKQARDSGEPFRPRWPVIILRTPKGWTGPRKIGDKYMEGYWRAHQVPITDVHENPGHLKLLERWMRSYEPERLFVDGRINPELRALCPTGNRRMSANPVANGGLLRKPLRMPDFRNYALEVEKPAVTMAASMQNMAKFLRDVVALNPTNFRLFGPDETESNKLAGVYQAGKKVWMGEYFEEDENGGNLAPNGRVMEILSEHTCEGWLEGYILSGRHGLLNSYEPFIHVIDSMVNQHCKWIEKCLEVEWRSKVASLNILLTAVVWRQDHNGFTHQDPGFLDVVANKSPEVVRIYLPPDGNCLLSCMDHCLRSSNYVNVIVADKQEHLQYLSMEDAIVHCTKGAGIWPQFSTDHGAEPDIVMASCGDIATHETLAAIDLLLQHFPELKIRYVNVVDLFRLISHIDHPHGMTDAEWEALFTADKPIIFNFHSYPWLVHRLSYKRPGAWRNLHVRGYKEKGNIDTPLELAIRNQTDRFSLAMDAIDRMAGSGVLGNRGAAAREALKNAQIRARTEAFENGVDPDFLKSWTWPYERTVQEAVPKLMG

SEQ ID NO: 34 - DNA, 2472 bp, Thermothelomyces heterothallica
atgggcgacgcaaatgagcttgagagcatcagcacctttggctctgcccgctcgaccatcaagggcgctcctctctcggaggaggaggtcaagaagtacaatgacttcttcaaggcgagtctgtatctcagcctcggcatgatatacctccgccataatccgctcctcaaggaacccctaaagaaggagcacctcaaagcccgactcctcggtcatttcggctcggcgcctgggcagatattcacctacatgcacttcaaccgcctcatcaacaagtatgaccttgacgccctcttcatatccggacccggccacggcgcccccgcggtgctctcgcaagcctacctcgagggcacctattccgaggtgtatcccgacaagtcggaggacgaggagggcctgcagaagtttttcaaacacttttcgtttcccggcggcattggctcgcacgcgaccccagaaactccaggcagcctgcacgagggtggcgagctggggtattcgatatcccacgccttcggcgccgtcttcgataaccccaatctgattgccctcaccatggtcggcgacggcgaggccgagacgggcccgctggcgacggcgtggcacagcaacaagttcctcaaccccatcaccgacggcgccgtgttgccggtcctgcatctcaacggctacaagatcaataaccccaccatcctcgcgcgcatcagccacaaggagctcgagaacctcttcctcggatacggctaccagccctactttgtcgagggtgacgaggtcgactcgatgcaccaggcaatggcagcgactcttgagcactgcgtgctggagatccgcaagtaccaaaagcaggccagggattccggcgagcccttccggcccaggtggccagttatcatcctccgcactccaaagggctggacgggtccgcgcaagatcggcgacaagtatatggaaggctactggcgcgcccaccaggtgccaatcacggacgtccacgagaaccccgggcacctcaaactactggagcgctggatgcgcagctacgagcccgagcggctcttcgtcgacggcaggatcaatccggagctcagggcgctctgcccgacggggaaccggcgcatgagcgccaatccggttgccaacggcggcctgcttcggaaaccgctcaggatgcctgactttcgcaactacgccctcgaggtggagaagccggccgtcaccatggctgccagcatgcaaaacatggccaagttcctgcgagatgtggttgccctaaatccgaccaacttccgcctgtttggtcccgacgagaccgaatccaacaagctcgccggggtgtaccaggcgggcaagaaggtctggatgggcgagtactttgaggaagacgagaacggcggcaacctcgcgcctaacggccgcgtcatggagattctctcggagcacacctgcgagggctggctcgagggctacatcctgagcggccgccatggccttctaaacagttacgagccgttcattcacgtcatcgactcgatggttaaccagcactgcaaatggatcgagaaatgcctcgaggtagaatggcgcagcaaggtcgcctcgctcaacatcctcttgaccgccgtggtctggcgacaggaccacaatggtttcacccaccaagacccgggcttcctagacgtggtggccaacaaaagccccgaagtggtgcgcatctacctgccgcccgatggtaactgcttgctgtcctgcatggaccattgcctccggtcgtccaattatgtcaatgtcatcgtcgccgacaagcaagagcacctgcaatacctctccatggaagacgccattgtgcactgcaccaagggcgccggtatctggccgcagttcagcaccgaccacggtgctgaaccggacatcgttatggcgtcctgcggcgacatcgccacccacgaaacgctagctgcgatcgatctccttctgcagcacttccccgagctcaagatccgctacgtcaacgtcgtcgacctcttcaggctcatctcgcacatcgaccacccacacgggatgactgatgccgagtgggaggccctcttcaccgccgacaaaccgatcatcttcaacttccacagctatccctggctagtccaccggctctcgtacaagcggcccggcgcgtggcggaacctgcacgtgcgcgggtacaaggagaagggcaacattgacaccccgctcgagctggcgatccgtaaccagacggaccgcttcagcctcgccatggacgccatcgatcggatggcaggcagcggggtgctcggaaaccgcggggccgcggccagggaggcgctcaagaacgcgcagatcagggcgcgaaccgaggcgttcgagaacggtgtcgacccggatttcttgaagagctggacatggccgtatgagcggaccgtgcaggaggctgtgccaaagctgatgggctga

SEQ ID NO: 35 - protein, 92 aa, Thermothelomyces heterothallica
MVKRVYFLVHGGVVQGVGFRYFTRHRAVELNLTGWVRNTDNNKVEGEAQGEDDAIATFLKHIDNGPRHAHVVKLDKEEREPVEGETEFEIRR

SEQ ID NO: 36 - DNA, 279 bp, Thermothelomyces heterothallica
atggtcaaaagagtatactttctcgtgcatggcggcgtggtgcagggggttggcttccgctacttcactcgccaccgggccgtcgagctcaatctgaccggttgggtgcggaacacggataacaacaaggtcgagggcgaagcccagggcgaagacgatgccattgcgaccttcctcaagcacatagacaacgggccacggcatgcccacgtggtcaagctggacaaggaggagagggagccggtggagggcgagaccgagttcgagattcgccggtga

SEQ ID NO: 37 - DNA, 5043 bp, Thermothelomyces heterothallica
cgcatgtgtcagccaatgtatttcgtttggtgaaaagataacgcgcgtcagacgaggggaaactgcctacccctgttcaccaccgacatgggcagactgtgtgccgagaagagcagcaccaccttgttcctcctctcctccggatactccagcaacttggcctcgatgttctgcgcaaacgcctcgacgaggcccgggtgtgtcggccaccggtcgatcacgctccacttgatcgtcccgtccgtcccgtcgttccgcagcgcggccttgccctccaacctctggcgccacttccacagctcgtttaggctactccccgtcgtcgagcacgagtactgcgggtactgggtgaatgcgacggcgcggcccccgcggccgttcccgaacccgtcggccaacatctgccggtacatgtcctcggtgagcgggttcgcgtagcggaaggcgacatagggcatatgcggtgccgtctcgggcgagatcttatcgaggattttgcacatctcggcgcactggtgctcggaccacttgcggatgggtgagccgccgccgatggccgcatattgttgctggatcttgggcgtgcgccgcttggagaggagcgggccgatgtagccctggagccggccgagagggatgagatcgccatcggactggaggtgaaacaaagcgttaggcgatcggtccgggcggccgtattttggagcaccggggggggggggggttaactcacgaatagtctgctgaggaagtcgcccacttcatcggtcgtcgatgggccgcccatgttgagaaacaccatagccgttgggccccgcccagagtcttgggtgactggatgaaccggcgtcgcgagccatcgtgcctgttgtgcagaaggttttgccagcctatgaggccggagtggccgcatggcggccgggagacgaagcgccatctcgaagcggaacacgggaatccgaggcgagttcgcagtaaaaaaagaaaaaaaaaatgaaaaagaagcgctgttagtcgttgcagtaaaaaagataaacaagaacaaacgggattgagacaatccctagggccatctatcaatttattcgcaatgcgtcagaggaaactgacgataccttggtttcagacagtggcgaacggaacaggaggccagatcacactccgcccgcgactttcgcggcaactcggcggcggtacgatcaaaggccgactttgccatcttggcatcggcgttgaccttgcagatcggccgggatcccttttggccaatcgcaaatgttcaattgcacagcttgccttgtcgtctgcgtcacatgttctggcgttaggcaggcgcgtcagcctagcatcacgtcgcgtcgcacctgcaccttcaaagcccgttggtcagcttcggcacgaacatgcccaacttctcgcccaaagccagaactttttagttttgtgcatcgatttggaaattacgtgcgcttgctaaattaaatttctgctcatcaaacaatggcgtgggaaggtgacgatgatagacgtccggattctgacgagggcgaggaggagctggatgaagccgtaggatcttccggtcagacgtaatgtcaatctccgctgactgcccatcaggattacaagacgcaaaaggatgcggtgctgtttgcgatcgacgtgagctcttcaatgctccaacagcccgttgcaacagatagcaagaaggcggacaaggattcggccatcacggcagccctgaaatgcgcataccagtttatgcaacagcgcatcatcgcgcaaccaaaggacatgatgggtatcctcctctttgggaccgaaaaatcaaagttccgtgatgaagctggagggcgcagcgggtctgggtatccccactgttacctcctgacggaccttgacgttcctactgccgaggacgtcaagagcctgaaggcgttggtggaagaaggggaggacccagacggagttctcgtccccgcgaaagagcccgcttccatggccaatgtgctcttctgcgccaaccaggtgtttaccaccaacgctcccaactttggctcccgaaggctgttcatcatcaccgatgacgacagtccacacgggaacgacaaggctgcgaaatcgtcggccgcggtacgcgccaaggatttgtatgaccttggtgttgtcatcgagctgttccccattagccacggagggaaggacttcgacatggccaagttttacgacgtatgtcgcccttttgctcccgactgcgtgtatgctaatttaacataccggcaggatattgtttaccgcgatccagcagccgaggcggggttcgtagaccgagtcaagacgtccaaatcaggagacggcatcagcctattgaactcgctgatctcgaacattaattccaagcagacgccaaagcgggcgtacttttccaacctgcactttgaacttgcgcctaacctcacgatatcggtcaagggttacttgcccctacacaggcaacaacccgcgcgcacgtgctacgtctggcttggcggcgagcgggcacagcttgcgcaatccgagaccgtaagagtcgactccacgacaaggactgttgacaagtccgaagtgaaaaaggcatataagttcgggggtgaatacatctacttcaagcccgaggaagcggcggcgttaaagaacctcggcagcaaagtcctccggctaatcgggttcaaaccacgctccctgctgcccatgtgggcctcagtgaagaagtccattttcatcttcccgagcgaggagcattatgtcggctccacccgtgtcttctccgcgctgtggcagaaactgctcgaggcggacaaggttgggatcgcatggttcgttgcccgcgagaatgcacatccctctatggtggccatcatcccttccagggcactggatgacgggtcttcggagacgccttacctcccggccgggctctggctgtacccgctaccgtttgcggacgatgtccgaaacgtggacttgacgatgccgccgagacccgccgacgagctcaccgacaggatgaggcagattgttcaaaaccttcagctgcccaaagccatgtacaacccatcgaaataccccaacccttctctacaatggcattacaaggtcttgcaggccatggcactggatgaggacgtgccggacagcctggacgacgcgacgatcccgaaatacaggcagatcgacaagcgcgtcggcgggtatctcgtcgagtggaaagaggtactcgccgagaaggccaatgcgctcatgaagagccgcgctgtgaagcgcgagtcggaggacgacggcggtgagcgcccggcagcgaagcgcaccaaggtcgcgccaaagaaggccgacaggggacagatgagcaacgcgcagcttaggactgctctggagcaggatacgctcaaaaagatgacggttgcggagttgagggacattctggctagtaaaggcatcagcgcagtgggcaaaaaggcggatctggttgagaagctggagcagtggatcgaggagaacatatgatcttgaaacggtttcttattctttggaatgtgtgtattgcagtcggtacgaagtatattctgtaatgatgctacttcgtcagggacatgcccttcccatggtttagcgttgctcaaaacacgttgttatccgagatgctctggagctgaagttccaaggcgtttttggagagagattgcggaactccaaacataaggtagagagagatattcctcagtccgcactaaacaaggtccctgtttaatagttacacagcaatggagatccatgcactcccgcacgtctggatgcacccacccttgctgctctctcggccccgctttggtctccttccactcattgccagttctgactggttcgcaacaacgcatgtcctcgtacgtccgcacgcagccactccactttacaatagaaactaaagatacccgcttggcaaagcgacacgacgacgcgacggagatactggtggtttgtcgcgccgtcctgttttctgatccaaacgacagccttgtcatggagactctgacctctgcattctgaagccaagcgaatgagcgcaggcgacccgacctacttgaaagagaacgagcggcaatggaggctctgctgggcaccggccagtcgaacccgacctgcggttcgctggccgacctccaggagcaactccggcatcttcttcagagtcgcgtgaccgaaactcgcgccgaacatatttcggtggcattcgaagtccgagcgaccgcgatttttgacatccctgtgaccggcgtcgaaaatgacctgctcgggaacccctcgaacatcgacccgtcgctgggcgggtcacggtcgagcgctgctgcgccagccatcaacggtagtgcgggacagccgacccgacgagtcagcgccatcgacgccctgatcaaccagcccgtggacgacccggtgttgcagactgcgattgccaggcagatcatatcgtcggtgggcgaggccgactcgagcaactgggcagtgcggcaggtctcgcgcgctgagcagagttggacgtttgcctacatctgcaaggattcctgggaggcctggaaccgtcaggcgtcgaagacacccgcgaagacgctcatcggggagtggagcggggagggtgggcaagatcccgttcatatgggtaggttcgttccacgctaccaaggcgagcgcgtgcgggttctgaccggatactgccagcacgtccggcctttgactgccgcggatccgtcaagatcgccttcgtcaaggcgacgaaaacgatcaacgtcaaatacgagcacactcccatgcacaagacggtggggcagctgatggaactccttgcgccgccacctgttgcaccaatcgtgaagaagacacccgtcaggaaggccaaggaagcaaagacgccaaaagaagcgagaccaccgaaggaaccgaagcccaagacgccaaggtcctccaagaagcgggcagaggtgaacggcgtcccgggcggagaaggttcgc

SEQ ID NO: 38 - DNA, 4189 bp, Thermothelomyces heterothallica
aacgagatgtacctacctggtatgtagggcatgaaatgtgtagcatctcgcgcatttaaccatatgtggtaatctgacgattgcttccactcgaggtcgaattgacctttcccagtttaaagatgtcgtgacaggggcggtaagtcgacgcagacagagctcaaactgggaaagccagaggaagggcggttcaagatagccagtaattccctggacactcaggaccgtcccgtcactctgactgagcgagcctgtcgatttgagacgatggggacgttgtctcgggctgggagtcccccatgaatcgagtcagtcttcagtcaatccagagaccgtttcggtggtaggttggaggaatgcccggggagtggaggctgtgcaggaaagaggtacatacctactatggagtagtatggagcttacacaacgagcggagccgcccccccagttgctctttcccgcacatgtgaaggtcatctccagaaaccaatcgaaggccaccaaagtattgaaagttgcaagctatttcccacccattacctttcaatcttttggggcctgctgcgagagagaaattcagtcaaagaaaagacgtccttcctctagtcgcttggtaagaaacgcaggctgtgggctgtgagagcgaggcacgcgttcacgcaagggcgggctggttgtatgcatgtatgtactgtacgtcttctgcagggcactatagacgggcaggtatgtactgtaccgtatgtcctgtacatacattcgcgcgggatgcgtaggggcctaactgtgtataccgtaccgcctactgaaaacccggaaaggcggggaatatccagatcttcgaaatacgcgaaacgccgaagccagcagcagcccgaagcatggaagactaccccaaagtaattgcgccagttcgccatgacccaaatcgcatgtcggctcggtcattgcacgtctttcaatattttgagggacagaaaagtcaccgacatcacaatcgttttgacatggtaggggaggtgctgaagatacggagtaccagtgacgtgaccgccatacccgctgaaaccccctcggtaccccctggcccgtgagcggggggccctgcaccgctcccgaagcctggaccgaggttgacggctacacagtcccgatatcacgggcgcacgagaccgggtcaaatacaagggacggcttctcttggtgggcgttgcattccaagtcgccatttcttttgctgttattttttttcttgctctttgtccggggccgggttttaccttacaagagctcgatctgcagtgttcgttgctcgctgcggcttgccgaggaaactgccgtcgccagtggaggggcgttttgcagagccgagaccgattcgtcgccaagcttgactctgaacttcgcacaatccgtccggatcgccgtgacaaccgccgaggccacgaccttgcgcgaggagcgcccccaagtcccatacgaggaccagaggatattgcaatggccgtacagtcaaaacctgcccccattgggccctggggcaaagcaacggccggcgccgcgggcgcagtcctcgccaatgcgcttgtgtaccctctcgatctgtacgtcgagcgtgacttagtcgccaccggcgctggcactggctaacggcatcgcggtctagcgtaaagacgaaacttcaggttcaagtaaaacccaccaacgccgaggggtccgactcaaaatccgccgagacgcactacaagggcacgtgggatgccatttccaagatcgcatccgccgagggcgtgacgggtttgtacgccggcatgggcggctcgttgattggtgttgcctcgacgaatttcgcctacttttactggtactcggtcgtccgcactctctacttcaagtacgccaaggcgacaggccagccgtctaccgtcgtcgaactgtccctcggcgccgtcgccggcgcgcttgcgcagctcttcacgatccctgttgccgtgatcacgacgcggcaacaaacgcagagcaaggaagagaggaagggcatcatcgacactgcgcgcgaggtgattgagggtgaggacggcatctcggggctctggagggggttaaaggcgagcctggtcttggtggtcaacccctccatcacctatggtgcgtatgagaggttaaaggatgttctgttcccgggcaagaagaacctctctccttgggaagctttcggtaagccaggccattgctgtggatgcttgcagcgttcaggaagtattgacaggcttgtctagctcttggggccatgtccaaggcactcgccaccatcgtcacgcagcccctgatcgtggccaaggtcggcctgcaatcgaagccgccaccggcgcggcaggggaagccgttcaagagtttcgtcgaggtgatgcagttcatcatcgcgaacgagggccccctgagccttttcaagggcatcgggccacagattctcaagggcctgttggttcagggtattctgatgatgaccaaggagcggtatgtggcgcggacccgcgtgtttggccaactgtactgacattgtgacagtgtcgaactcatgtttatcctcttcgttcgttatctccaagtcatgcggtcgcgcagacctgggaaggccgtcgatctcgcggctgccgccaagctcgtcgctcccgtcacggtcaaataatcactattaccttgctttagccggggttgtttcattgtggtgcctcgctgcaaagtctgaaatgctgtcactgtacaagaagcacggctggggcctgtggagcggttgtcgggaagggtttctgctttgtacatggatacctgcatatagattcttcattttctttctttctggcataatgaggattttgcatgattgaatgaagagtcgagtcctaattatggttgatgtgttgatggagtggttagacgtacggattactgtgcaaggaacaaccccttcgccgttacacactacgcaatacagtggttctggctcgaacgttcacgtattaagccacaactcgagccccaccctagccacacccaccagattagggcagccaaatttctcccacttttgtgcgcaagtgacttccttcggttgttgaaccttgcaccccagcttcttcagactcagtcatcgtcagtcggagcccaaatccatcacaatggccagtacgtcgcgtttcatattccttgaaaggctgaattattcacatactgacaatttgcagagtccaagaactcgtctcagcacaaccagtcgcgcaaggcgcaccggaacgggtacgcaacacgagccaccgatacggcaccgcaatgagaacctggatgctaacagcgagatcgcaacagcatcaagaagcccaagacccagaggtacccatccctcaagggtaccgatcccaagttccggagaaaccacaggcatgcgctccacggcaccgctaaggctatcgtacgacccgaccgcccgtcacacctgcaatgcaagaagcgacctgactaacacaatttatagaaagagttcaaggagggcaagagggagacctgctaaattttattcttgtttgctggcggcgcacgcgatacggttggagaggacacatcttcgggcatgtccttgacgaagaagggattctaggcggcgacgatatttttttctctgccccgccgattactgggccggttgttcgaggtgttatttagggcgtcatttcgatcaacccaaacggaatgcatacgacctccgcgcagagaagcggatctgccggggtgctaccctcttcccgattgcgggcacagctcgattggggccggaccggcccaagcaagactgtcaaggtttgtgatgctgattgtgtgtcgagcgactgagggcgacaagcccgacatccttcctttgcgttgcacttttcaatcgtttctgagtgctttctctctttggggcatcatgacagcgaaaataaacggcattagtgtgtacagctctgagcttcttcccatgctcattctgctgaaggattatatcgtgccaagatggacctgcctcactattaccaaacacccctgatcttcgtccgaaaggctcgggtgcatgaaagcgcggctacatccacctttatacattttcatcagcttcggcgccggtgccttctctaaagagccagaagcttgc

SEQ ID NO: 39 - DNA, 8017 bp, Thermothelomyces heterothallica
agggtaggtgggatgggcggggtgtagggtaggtcggtgtagggtaggtcggctgggcggggtgtagggtaggtcggttgggcggggtgtagggtaggtcggttgggatgggtgtagggtaggtcggccgggtgtagggtaggtcggctgggcggggtgtagggtaggtcggtgtagggtaggtgggatggggcgctatgtgcggccgcgagctcgcgagcccatttttagcgaaggccatacaaacgagttttgcggaacccgggattccacccccgaagccgccggcgcgtgcgccccgctgcgcatcggtcggtgggtatatgagaagggggcgggcaagccggaagccagaggcaactgctactgttagctgccgctggcctccgcggcccagggcgcggcacggctgcgttgaagtctcccagtctcccacccgttggctgcgcggatccgcccgtcttggtggttgcgagctcgcgagcccatttttagcgaaggccatacaaacgagttttgcggggcccgggattccaccccggaacccgccggcgcgtgcgccccgctgcgcatcggtcggtgggtatgtgagggaggaagaagaaaaaaaaaaaaagctcctgcgggggggctgtcgggcacgcctactttcgggcgacccggcacctctccgcggcagccttcgcaggccgctgttggtcccatttcatacgtcgccgccttcgcgtggtgccctacggtctgccggggtaccgacgattgcggcgagcaccgcctcagcaccgctgctgccaccggcgcgacctcgcccgggggtgcgcgcggcatctgggaagactctgcaggcgtaagggaataccccatgtgcgccgaggggtgggctatgtgggtgcttggcggttcgccagacctttctaaagccaccgggggtacctaccggttggggacgcctacagggctgaacctcccggtcgggcctcctcttggggcgcttaggcggcgacttcggggcgcgatcgctccccgctctcgcccgccgacggcgctctggggaattcaggaggggaaagcagatgtgacccgcggctcgaccggcgcattgccggacgagctgcgcggccacgcgggcccccgcgcccgccgacccagtaacttagtgaactcttccgccctgaaacacgggcggttggccctaaccggctcacgatagttacctggttgattctgccagtagtcatatgcttgtctcaaagattaagccatgcatgtctaagtataagcaattatacagcgaaactgcgaatggctcattaaatcagttatcgtttatttgatagtaccttactacatggataaccgtggtaattctagagctaatacatgctaaaaatcccgacttcggaagggatgtatttattagattaaaaaccaatgccctccggggctctctggtgattcatgataacttctcgaatcgcacggccttgcgccggcgatggttcattcaaatttctgccctatcaactttcgacggctgggtcttggccagccgtggtgacaacgggtaacggagggttagggctcgaccccggagaaggagcctgagaaacggctactacatccaaggaaggcagcaggcgcgcaaattacccaatcccgacacggggaggtagtgacaataaatactgatacagggctcttttgggtcttgtaattggaatgagtacaatttaaatcccttaacgaggaacaattggagggcaagtctggtgccagcagccgcggtaattccagctccaatagcgtatattaaagttgttgaggttaaaaagctcgtagttgaaccttgggcctagccggccggtccgcctcaccgcgtgcactggctcggctgggtctttccttctggagaaccgcatgcccttcactgggtgtgccggggaaccaggacttttactctgaacaaattagatcgcttaaagaaggcctatgctcgaatacattagcatggaataatagaataggacgtgtggttctattttgttggtttctaggaccgccgtaatgattaatagggacagtcgggggcatcagtattcaattgtcagaggtgaaattcttggatttattgaagactaactactgcgaaagcatttgccaaggatgttttcattaatcaggaacgaaagttaggggatcgaagacgatcagataccgtcgtagtcttaaccataaactatgccgattagggatcggacggcgttattttttgacccgttcggcaccttacgataaatcaaaatgtttgggctcctgggggagtatggtcgcaaggctgaaacttaaagaaattgacggaagggcaccaccaggggtggagcctgcggcttaatttgactcaacacggggaaactcaccaggtccagacacgatgaggattgacagattgagagctctttcttgatttcgtgggtggtggtgcatggccgttcttagttggtggagtgatttgtctgcttaattgcgataacgaacgagaccttaacctgctaaatagcccgtattgctttggcagtacgccggcttcttagagggactatcggctcaagccgatggaagtttgaggcaataacaggtctgtgatgcccttagatgttctgggccgcacgcgcgctacactgacagagccagcgagtactcccttggccggaaggcccgggtaatcttgttaaactctgtcgtgctggggatagagcattgcaattattgctcttcaacgaggaatccctagtaagcgcaagtcatcagcttgcgttgattacgtccctgccctttgtacacaccgcccgtcgctactaccgattgaatggctcagtgaggctttcggactggcccagagaggtcggcaacgaccactcagggccggaaagttatccaaactcggtcatttagaggaagtaaaagtcgtaacaaggtctccgttggtgaaccagcggagggatcattacagagctgcaaaactccctaaaccatcgtgaacgctacctagaccgttgcttcggcgggcggcgccctcgcgcgccccccctggggcccgcaccgcgggcgcccgccggaggtacaccaaactcttgatatgttatggccactctgagtctcctgtactgaataagtcaaaactttcaacaacggatctcttggttctggcatcgatgaagaacgcagcgaaatgcgataagtaatgtgaattgcagaattcagtgaatcatcgaatctttgaacgcacattgcgcccgccagcatcctggcgggcatgcctgttcgagcgtcatttcaacccatcaagcccacggcttgtgttggggacctgcggctgcccgcaggccctgaaaaccagtggcgggctcgctagtcacaccgggcgtagtagcatacgacctcgctcagggcgtgctgcgggttccagccgtaaaacgaccttcacaacccaaggttgacctcggatcaggtaggaggacccgctgaacttaagcatatcaataagcggaggaaaagaaaccaacagggattgccctagtaacggcgagtgaagcggcaacagctcaaatttgaaatctggcttcggcccgagttgtaatttgcagaggaagctttaggcgcggcaccttctgagtcccctggaacggggcgccatagagggtgagagccccgtatagttggatgcctagcctgtgtaaagctccttcgacgagtcgagtagtttgggaatgctgctcaaaatgggaggtaaatttcttctaaagctaaataccggccagagaccgatagcgcacaagtagagtgatcgaaagatgaaaagcactttgaaaagagggttaaatagcacgtgaaattgttgaaagggaagcgcttgtgaccagacttgcgccgggctgatcatccggtgttctcaccggtgcactctgcccggctcaggccagcatcggttctcgcggggggataaaggcccggggaatgtagctcctccgggagtgttatagccccgggtgtaataccctcgcggggaccgaggttcgcgcatctgcaaggatgctggcgtaatggtcatcagcgacccgtcttgaaacacggaccaaggagtcaaggttttgcgcgagtgtttgggtgtaaaacccgcacgcgtaatgaaagtgaacgtaggtgagagcttcggcgcatcatcgaccgatcctgatgttttcggatggatttgagtaggagcgttaagccttggacccgaaagatggtgaactatgcttggatagggtgaagccagaggaaactctggtggaggctcgcagcggttctgacgtgcaaatcgatcgtcaaatctgagcatgggggcgaaagactaatcgaaccatctagtagctggttaccgccgaagtttccctcaggatagcagtgttgtcttcagttttatgaggtaaagcgaatgattagggactcgggggcgctttttagccttcatccattctcaaactttaaatatgtaagaagcccttgttacttagttgaacgtgggccttcgaatgtatcaacactagtgggccatttttggtaagcagaactggcgatgcgggatgaaccgaacgcggggttaaggtgccggagtggacgctcatcagacaccacaaaaggcgttagtacatcttgacagcaggacggtggccatggaagtcggaatccgctaaggactgtgtaacaactcacctgccgaatgtactagccctgaaaatggatggcgctcaagcgtcccacccataccccgccctcagggtagaaacgacgccctgaggagtaggcggccgtggaggtcagtgacgaagcctagggcgtgagcccgggtcgaacggcctctagtgcagatcttggtggtagtagcaaatacttcaatgagaacttgaaggaccgaagtggggaaaggttccatgtgaacagcggttggacatgggttagtcgatcctaagccatagggaagttccgtttcaaaggggcactcgtgccccgtgtggcgaaagggaagccggttaacattccggcacctggatgtgggttttgcgcggtaacgcaactgaacgcggagacgacggcgggggccccgggcagagttctcttttcttcttaacggtctatcaccctggaaacagtttgtctggagatagggtttaacggccggaagagcccgacacttctgtcgggtccggtgcgctctcgacgtcccttgaaaatccgcgggagggaataattctcacgccaggtcgtactcataaccgcagcaggtccccaaggtgaacagcctctggttgatagaacaatgtagataagggaagtcggcaaaatagatccgtaacttcgggataaggattggctctaagggttgggcacgttgggctttgggcggacgccctgggagcaggtcgcctctagccgggcaaccggcggggggcttccagcatccgggtgcagatgcccttagcaggcttcggccgtccggcgcgcggttaacaaccaacttagaactggtacggacagggggaatctgactgtctaattaaaacatagcattgcgatggccagaaagtggtgttgacgcaatgtgatttctgcccagtgctctgaatgtcaaagtgaagaaattcaaccaagcgcgggtaaacggcgggagtaactatgactctcttaaggtagccaaatgcctcgtcatctaattagtgacgcgcatgaatggattaacgagattcccactgtccctatctactatctagcgaaaccacagccaagggaacgggcttggcagaatcagcggggaaagaagaccctgttgagcttgactctagtttgacattgtgaaaagacataggaggtgtagaataggtgggagcttcggcgccggtgaaataccactactcctattgtttttttacttattcaatgaagcggggctggattttcgtccaacttctggttttaaggtccttcgcgggccgacccgggttgaagacattgtcaggtggggagtttggctggggcggcacatctgttaaaccataacgcaggtgtcctaaggggggctcatggagaacagaaatctccagtagaacaaaagggtaaaagtccccttgattttgattttcagtgtgaatacaaaccatgaaagtgtggcctatcgatcctttagtccctcgaaatttgaggctagaggtgccagaaaagttaccacagggataactggcttgtggcggccaagcgttcatagcgacgtcgctttttgatccttcgatgtcggctcttcctatcataccgaagcagaattcggtaagcgttggattgttcacccactaatagggaacgtgagctgggtttagaccgtcgtgagacaggttagttttaccctactgatgaactcatcgcaatggtaattcagcttagtacgagaggaaccgctgattcagataattggtttttgcggttgtccgaccgggcagtgccgcgaagctaccatctgctggataatggctgaacgcctctaagtcagaatccatgccagaacgcgatgatactacccgcacgttgtagacgtataagaataggctccggcctcgtatcctagcaggcgattcctccgccggcctcgaagttggccggcggtaattcgcgtattgcaatttcgacacgcgcgggatcaaatcctttgcagacgacttagatgtgcgaaagggtcctgtaagcagtagagtagccttgttgttacgatctgctgagggtaagccctccttcgcctagatttcccagcgagagcccgccggcggaacagccgggcgagccttacgggggaagccttaaggggattgagaagtggtgccgtgcgttcgcgcgcccctaggtcctttagccggccgcaggtgtagggtaggtcggttgggaggatggggtgtagggtaggtcggtgtagggtaggttggttgggaggatggggtgtagggtaggtcggccgggtgtagggtaggtcggtgtagggtaggtgggatggggcgctatatgcggccgcgagctcgcgagcctatttttagtgaaggctatataaataagctttacgttaccgggccttgctaccctcgagtggcgtgggccgtgctgcctactgggcattgctcgccgggctgtataagggaggggtcggggtcgcggtctagggtaggtcgggtgggatggggtgtagggtaggagaagcgctctagtcgtgtgtctttttctctaggtctattattagtactggctgtagggcgacgtgccctgccttgttataatattatattgtatgtttaggcctatactagcttgtaatctatttgtatctggcttattaggtacggcttcctttgtatataactagagaggctctggtatgcttcttagtatagcggtataggattcataatcatagtaatgataatcataatagtaataataataataatagtaatgataataataataatctatttatatcttatttaaaatgcttgtacggctgcctgctcttaaggagtagctagatatgagatggtagggtagctagctaacctaggctagacgttctcgtcccttagctatataagtgctatatattatagttagttatctaacctaccttcttacttgagcagaagaggtagggttctagtatagctagtagggcttctaggcctaagggcctgttattcgagttattataggttagtatttaatatagttatagggataggcctcgattacgggtataggataggtaggataggtatagggtaggtcggttaggaggatagggtgtaaggtaggtcggccgggtatagggtaggtagtaggttaggcggggtgtagggtaggtcggtgtagggtaggtgggatgggcggggtgtagggtaggtcggttgggaggatggggtgtagggtaggtcggtgtagggtaggtcgg

SEQ ID NO: 40 - DNA, 13720 bp, Artificial Sequence
  Synthetic Polynucleotide
aaaccccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtatttaaatctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctggcgcgcccatttccacaactcatgccgagagaagtgtcagactgggcaactaaagtagtagtagtaatagctcgattaccatgatgaaatgctgggcgtcgaagcagctgcaggtccggcatgcagcaatccccactccgctccatcggctgctgttctggggtcaatccgtaccctcccaagttcacctcgccgctgacctcgggatcagctgcgttgtgcattcatgaataatgcgcaacatgagcaacccaacttcatcaagggagtttcgacgtcaccatatcaccacacatcttggaacagaattggggacaaggcagctggattgacggggaaatacataaaccggacgacctatgacgaccatcctatcccgtcaccgactccgatcccctgcggatgatggctcaaagaaccaagtttgatgagccggctgtgtgtcccagcacatacaacaaccgaagtatcagccccgttccgaacgcaggatcccagtctaccgaatcgattttggacagcccgagagaagccaaacaccgaaccgaaggaggaattgtcggaagacgtgcattatgagtcgttcacgcgcgcgttagtggcggtggcgcgggaggggcgggatgctctgctcgttgcggaagaagttgttggggtcgacgagggtcttgaccttgaccaggcggtcgaagttcttgccgaagtacttctcgccccagatgcgggcctgggtgtagttgttcgggttcttggggtcgttgatgccgatgtcgaggtcgcggtagttgaggtaggccaggcgcgggttcttgctgacgtagggggtcatgaagttgtagatgttgcggatccagttcaggtgcttctcgttgtcttcctgcttctcccaggagcagatgtaccagagctcgtacaggatgccggcgcggtgggggaaggggatggccgactcggagatctcgtccatgatgccaccgtacgggtagagggcgtacatgccggcgccgatgtcttcctcgtagagcttctccaggatctggacgaagacggactccgggatgggcttcttgacgtagtccagcttgatcttgaaggcgccgttctggccggcggagcggtccagcaggatctccttgttgaagttgtcggtgtcgtagttgacgacgcccgagtagaagatgatggtgtcgatccagctgagctggcggcagtcggtcttcttgatgcccagctccgggaaggacttgttcatgaggtcgaccagggagtcgacgccgccgaggaagacggacgagaagtaggtgtggatggcggtcttgttcttgccctggttgtcggtgatgttgcgggtgatgaagtgggtcatgagcaggaggtccttgtcgtacttgtaggcgatgttctgccacttgttgacgagcttgaccagctcgtggatctccatgatcttcttgacggagaacatggtcgacttggggacggcgaccaggcggatcttccaggcgacgatgatgccgaagctctcggcgccgccgcccctgagggcccagaacaggtcctcgcccatggacttgcggtcgaggaccttgccgtggacgttgaccaggtgggcgtcgatgatgttgtcggcggcgaggccgtagttgcgcatcagggggccgtagccgccgccaccgaagtggccgccagcgcagacggtggggcagtagccggcggccagggacaggttctcgttcttctcgttgacccagtagtagacctcgccgagggtggcgccggcctcgacccaggcggtctggctgtggacgtcgatcttgatggagcgcatgttgcgcaggtcgacgatgacgaaggggacctgcgagatgtagctcatgccctccgagtcgtggccgccgctgcgggtgcggatctggaggccgaccttcttcgagcacaggatggtgccctggatgtgggagacgtgcgacggggtgacgatgacgagcggcttgggggtggtgtcgctggtgaagcgcaggttgtggatggtgctgttgaggacggacatgtacagggggttgttctgggtgtagacgagcttcaggttggtggcgttgttcgggatgtactgcgagaagcacttgaggaagttctcgcgggggttcatgttgcttgcgtggtgtgctggaagctgagtgtattaggtggattgacaagtccctgcgggcaacgggaccgagtgagcaagccaggatcaggcgagcaagaggcaggtggtctgattctatcaacctacgtttagagacttgagatggaccagggaatgggcgttttgttttcgaattgatggtttacgatggatttcgttggacggaagaccgatgaggggaaaggagaggagaagcccaaagagggggtcggaggtggcctttattaagaggcggccggccgggcaatgggcagatcagccatctttgctgcatcgttcctgcgactgtttcgtcagaccgggcggggtaatgtcaggagagctccctggtaggttgcgggcagcggcagtgatcacgttgactggctcacgggatcgcgtgacgagtacatcatgatggcacgacctcgcaggcgagccctgcgtggcttaaacaggccaaggtacctggcccgagcgtcctgcagccagcgctaacagcccagcccaagccagaatctggggtaatctggggtaccggggtgcccgacccactgcgggcaaccagcgcttgtgcaccgcgtaaggcctcaacaagacatcagttagtatcgatgccgagattcagttggcaattacatacgtctaacttttccaatgcttattttgagtttcttgtagttatgcagctggtggaagttaggacaggaaccttgagtgacaagcaatccggccgggccgggaaggtgcccgctgcacgatcagtggggcaaaggtggggtatcccgagcaggagcgaactccaacagagtattcgatcaaaaaaggcaagtcctcccccaccatcctttgtagcttgcaatgcatctccttttttgcaatggattttgcttcgcgagtgctaatgccttgtgaaggactatgtggttggttcaaacctgttgttttgatccatctagtccacgttgcaggcatacaaatacccgacgacgtgtatcataagttaagtaggtactgtacgttagtttgtttaccttcttcagccagtagtccgactttgctctcaagtgctcatccaaccctttggccttccaaatcttgataccgagagcctcaggcaggttggtatattctacctcaaatacctgccagatgttgaccaatgccgttgtcgttactccgtactacgcgaaccaatgccaacatgattctcctttcagtcacgagacgattcgagtgtcatggtagcagtactttggtagtaaagagtcactcaattgaacatgtcgtagctcattcgtaaccaagtcatgataaccctgaatttcaggggttcccttcagagacaggagccgtcatgttgcggcaagagaaaaaaaaatgaagaggaaacacacaaaaacgtgggaagaatagccatcagcggctaccctcatactccaactcctgcctgccttaattaattacttgcgcggggtgtagtcgaagatcaggagcttctcccagaacgagcggtagacgtcgccgaagccgacgtgggccgggtggatgatgtagtcctggatcgtctcgacgctctcgaaggtgacctcgacgatgtgggtgtagccctcttccttgttcttctgggtgacgtccttgccccagtagacgtccttcatggcggggatgatgttgacgaggttgacgtaggtcttgaagaactcttccttctgggcctcggtgatctcgtccttgaacttcaggacgatgaggtgcttgacggccatggtgtgagggactaggtaggcttttgtagtgttgcagggagttggaggggatgtgcgacttctggtttgcttctcttaggttgagtatgaaagttgaggacgactcggggtcaagacgtcctgagagagagcgggagcatgctggcccccgacgccttcttaagcaaaccagctcatggcgcgtcagactggattcggaagatcgaccgggaacgagtaagggccagtggttggcacctctcgggcagcagcagcagcagcagcagcagcaatgtgccatggcatctgcgcgatcgggcatcgttgaccgctgttcccgcaggcgatgtaccatgttatccgcgcctgcctgcttgcgagtggtgccatggcaaatgctggaagcgggtccctcgctacagagtaaatccacggctgcaggagacgcgcagttggtcatccctggggcccctgcgccacgcggcactgccttacccctctgcacacgcgtgactaacccccactactgagtaccccgcttgtcaaaaggtcgcttccatacttatcgccagcctgacattatcgcgtctgcactggaaacctaagcgggtaaagcatcagagcatcaaatccaaggctctttctcctatctctgtaaatgagaggacaagttgatttcggaatcccgagtagaacggcagacagccaggcatactatcattacgcagctccggggaaagatccgacaaccagagccagtctctttctgccgttctgatgattccatcttcccctcagctccttcaccgcccagcgtctgctacgtgtccggcccggctttgcctgcctcgtcctgcagccaacgggactgcgcgaccgagccgccgactctgcaagtaatggtacctaacgaccgccccaagctggtagctctgtcctggtttcgccgcgtaagtctcggcgctagccttgattatgctgtctcggatctggaccaagtgtttcgatattccatcccatgacctacatgcgccggcgagacgcactacaagggcacgtgggatgccatttccaagatcgcatccgccgagggcgtgacgggtttgtacgccggcatgggcggctcgttgattggtgttgcctcgacgaatttcgcctacttttactggtactcggtcgtccgcactctctacttcaagtacgccaaggcgacaggccagccgtctaccgtcgtcgaactgtccctcggcgccgtcgccggcgcgcttgcgcagctcttcacgatccctgttgccgtgatcacgacgcggcaacaaacgcagagcaaggaagagaggaagggcatcatcgacactgcgcgcgaggtgattgagggtgaggacggcatctcggggctctggagggggttaaaggcgagcctggtcttggtggtcaacccctccatcacctatggtgcgtatgagaggttaaaggatgttctgttcccgggcaagaagaacctctctccttgggaagctttcggtaagccaggccattgctgtggatgcttgcagcgttcaggaagtattgacaggcttgtctagctcttggggccatgtccaaggcactcgccaccatcgtcacgcagcccctgatcgtggccaaggtcggcctgcaatcgaagccgccaccggcgcggcaggggaagccgttcaagagtttcgtcgaggtgatgcagttcatcatcgcgaacgagggccccctgagccttttcaagggcatcgggccacagattctcaagggcctgttggttcagggtattctgatgatgaccaaggagcggtatgtggcgcggacccgcgtgtttggccaactgtactgacattgtgacagtgtcgaactcatgtttatcctcttcgttcgttatctccaagtcatgcggtcgcgcagacctgggaaggccgtcgatctcgcggctgccgccaagctcgtcgctcccgtcacggtcaaataatcactattaccttgctttagccggggttgtttcattgtggtgactagtgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgggcgcgccgttt

SEQ ID NO: 41 - DNA, 14329 bp, Artificial Sequence
  Synthetic Polynucleotide
aaaccccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtatttaaatctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctggcgcgcccatttccacaactcatgccgagagaagtgtcagactgggcaactaaagtagtagtagtaatagctcgattaccatgatgaaatgctgggcgtcgaagcagctgcaggtccggcatgcagcaatccccactccgctccatcggctgctgttctggggtcaatccgtaccctcccaagttcacctcgccgctgacctcgggatcagctgcgttgtgcattcatgaataatgcgcaacatgagcaacccaacttcatcaagggagtttcgacgtcaccatatcaccacacatcttggaacagaattggggacaaggcagctggattgacggggaaatacataaaccggacgacctatgacgaccatcctatcccgtcaccgactccgatcccctgcggatgatggctcaaagaaccaagtttgatgagccggctgtgtgtcccagcacatacaacaaccgaagtatcagccccgttccgaacgcaggatcccagtctaccgaatcgattttggacagcccgagagaagccaaacaccgaaccgaaggaggaattgtcggaagacgtgcattatgagtcgttcacgcgcgcgttactcgaagtggctgaactgctggcggaggaccctgcgcatgatcttgttggtggcggtgcggggcagggaggagagggggacgacgcgggtgaccttgaacagcgggttgagcttcttctgcaggccgaggttgaacgacaggcggagctggttgaggtcgatggtggtgtcgttgctgtccttgaggacgaagaagatgaccagctgctcggggccgccgccgaggggcgggacgccgatggcggtcgtctcgaagacgcggtcgtcgacctcgttgcagacgcgctcgatctcgatgctggagatcttgatgccgccgatgttcatggtgtcgtcggcgcggccgtgggcgtggtagtagccgttggaggtgagctcgaagatgtcgccgtgcctgcggaggacctcgccgttgagggtgggcatgcccttgaagtagacgtcgtggtggttgccgttcaggagggtcttcgaggcgccgaacatgaccgggcccagggccagctcgccgatgcccggcttgttcttcggcatggggtagccgttcttgtccaggatgtagagggtgcagcccatgcactgcgagctgaaggacgagagggactgggcctgcaggaaggagccggcggagaaggcgccgccgatctcggtgccaccgcacatctcgatgacgggcttgtagttggcgcggcccatcagccagaggtactcgtcgacgttgctggcctcgcccgacgaggagaagcagcggatggtgctccagtcgtagccggagacgcagttggtgctcttccaggagcggacgatggaggggacgacgcccagcatggtgaccttggcgtcctggacgaacttggcgaagcccgagacgagggggctgccgttgtacagggcgatggaggcgccgttcaggagggaggcgtagaccagccaggggcccatcatccagcccaggttggtgggccagacgatgacgtcgcccttgcggatgtcgaggtgcgaccagccgtcggcggcggccttcagcggggtggcctgggtccaggggatggccttgggctcgccggtggtgcccgaggagaagaggatgttggtgtaggcgtcgaccggctgctcgcgggcggtgaactcgcagttcttgaactccttggcgcgctcgaggaagtagtcccagctgatgtcgccgtcgcgcagctcggcgccgatgttcgagccggagcaggggatgacgatggccatgggggacttggcctcgacgacgcgcgagtacagcgggatgcgcttcttgccgcggatgatgtggtcctgggtgaagatggccttggccttgctcaggcggaggcgggtggagatctccggggccgagaacgagtcggcgatgctgacgacgacgtagccggccaggacgatggcgaggtagatgacgacggcgtcgacgtgcatcggcatgtcgatggcgatggcgcagcccttctcgaggcccatctcctccagggcgtagccgaccagccagacgcgcttgcgcagctggtcgagggtgagcttgttcagggggaggtcgtcgttgccctcgtcgcgccagacgatcatggtgtcgttgagcttcttgttggagttgacgttcaggcagttcttggccgagttcaggtagccgccagggagccactccgagccgccagggttgttgatgtcgtccctgcggaggatgcactcggggtccttcgagaagctgatcttcatctcgtccatcaggacggtgcgccagtagacctccgggttgcggaccgagaactcctggaagtggctgaacgagctgatggggtccttgtacttgacgccgaggaactccttgccgcgcttctccaggagggcgcccaggttggtggacttgaccttctcggggtccgggatccaggcgggaggggcggggccgaagtccttgtagcagccgtagaacagcatctggtggagggagaacggcaggtcgggcgagaggatgtggttggcgatgttgatccaggtctgcggggtggcggcgccgtagttgcagacgatctcggccaggcggccgtggagcgtctcggcgacctccgaggtgatgcccagggcgatgaagtcggaggcgacgaccgagtccaggctcttgtagttcttgcccatgttgcttgcgtggtgtgctggaagctgagtgtattaggtggattgacaagtccctgcgggcaacgggaccgagtgagcaagccaggatcaggcgagcaagaggcaggtggtctgattctatcaacctacgtttagagacttgagatggaccagggaatgggcgttttgttttcgaattgatggtttacgatggatttcgttggacggaagaccgatgaggggaaaggagaggagaagcccaaagagggggtcggaggtggcctttattaagaggcggccggccgggcaatgggcagatcagccatctttgctgcatcgttcctgcgactgtttcgtcagaccgggcggggtaatgtcaggagagctccctggtaggttgcgggcagcggcagtgatcacgttgactggctcacgggatcgcgtgacgagtacatcatgatggcacgacctcgcaggcgagccctgcgtggcttaaacaggccaaggtacctggcccgagcgtcctgcagccagcgctaacagcccagcccaagccagaatctggggtaatctggggtaccggggtgcccgacccactgcgggcaaccagcgcttgtgcaccgcgtaaggcctcaacaagacatcagttagtatcgatgccgagattcagttggcaattacatacgtctaacttttccaatgcttattttgagtttcttgtagttatgcagctggtggaagttaggacaggaaccttgagtgacaagcaatccggccgggccgggaaggtgcccgctgcacgatcagtggggcaaaggtggggtatcccgagcaggagcgaactccaacagagtattcgatcaaaaaaggcaagtcctcccccaccatcctttgtagcttgcaatgcatctccttttttgcaatggattttgcttcgcgagtgctaatgccttgtgaaggactatgtggttggttcaaacctgttgttttgatccatctagtccacgttgcaggcatacaaatacccgacgacgtgtatcataagttaagtaggtactgtacgttagtttgtttaccttcttcagccagtagtccgactttgctctcaagtgctcatccaaccctttggccttccaaatcttgataccgagagcctcaggcaggttggtatattctacctcaaatacctgccagatgttgaccaatgccgttgtcgttactccgtactacgcgaaccaatgccaacatgattctcctttcagtcacgagacgattcgagtgtcatggtagcagtactttggtagtaaagagtcactcaattgaacatgtcgtagctcattcgtaaccaagtcatgataaccctgaatttcaggggttcccttcagagacaggagccgtcatgttgcggcaagagaaaaaaaaatgaagaggaaacacacaaaaacgtgggaagaatagccatcagcggctaccctcatactccaactcctgcctgccttaattaattacttgcgcggggtgtagtcgaagatcaggagcttctcccagaacgagcggtagacgtcgccgaagccgacgtgggccgggtggatgatgtagtcctggatcgtctcgacgctctcgaaggtgacctcgacgatgtgggtgtagccctcttccttgttcttctgggtgacgtccttgccccagtagacgtccttcatggcggggatgatgttgacgaggttgacgtaggtcttgaagaactcttccttctgggcctcggtgatctcgtccttgaacttcaggacgatgaggtgcttgacggccatggtgtgagggactaggtaggcttttgtagtgttgcagggagttggaggggatgtgcgacttctggtttgcttctcttaggttgagtatgaaagttgaggacgactcggggtcaagacgtcctgagagagagcgggagcatgctggcccccgacgccttcttaagcaaaccagctcatggcgcgtcagactggattcggaagatcgaccgggaacgagtaagggccagtggttggcacctctcgggcagcagcagcagcagcagcagcagcaatgtgccatggcatctgcgcgatcgggcatcgttgaccgctgttcccgcaggcgatgtaccatgttatccgcgcctgcctgcttgcgagtggtgccatggcaaatgctggaagcgggtccctcgctacagagtaaatccacggctgcaggagacgcgcagttggtcatccctggggcccctgcgccacgcggcactgccttacccctctgcacacgcgtgactaacccccactactgagtaccccgcttgtcaaaaggtcgcttccatacttatcgccagcctgacattatcgcgtctgcactggaaacctaagcgggtaaagcatcagagcatcaaatccaaggctctttctcctatctctgtaaatgagaggacaagttgatttcggaatcccgagtagaacggcagacagccaggcatactatcattacgcagctccggggaaagatccgacaaccagagccagtctctttctgccgttctgatgattccatcttcccctcagctccttcaccgcccagcgtctgctacgtgtccggcccggctttgcctgcctcgtcctgcagccaacgggactgcgcgaccgagccgccgactctgcaagtaatggtacctaacgaccgccccaagctggtagctctgtcctggtttcgccgcgtaagtctcggcgctagccttgattatgctgtctcggatctggaccaagtgtttcgatattccatcccatgacctacatgcgccggcgagacgcactacaagggcacgtgggatgccatttccaagatcgcatccgccgagggcgtgacgggtttgtacgccggcatgggcggctcgttgattggtgttgcctcgacgaatttcgcctacttttactggtactcggtcgtccgcactctctacttcaagtacgccaaggcgacaggccagccgtctaccgtcgtcgaactgtccctcggcgccgtcgccggcgcgcttgcgcagctcttcacgatccctgttgccgtgatcacgacgcggcaacaaacgcagagcaaggaagagaggaagggcatcatcgacactgcgcgcgaggtgattgagggtgaggacggcatctcggggctctggagggggttaaaggcgagcctggtcttggtggtcaacccctccatcacctatggtgcgtatgagaggttaaaggatgttctgttcccgggcaagaagaacctctctccttgggaagctttcggtaagccaggccattgctgtggatgcttgcagcgttcaggaagtattgacaggcttgtctagctcttggggccatgtccaaggcactcgccaccatcgtcacgcagcccctgatcgtggccaaggtcggcctgcaatcgaagccgccaccggcgcggcaggggaagccgttcaagagtttcgtcgaggtgatgcagttcatcatcgcgaacgagggccccctgagccttttcaagggcatcgggccacagattctcaagggcctgttggttcagggtattctgatgatgaccaaggagcggtatgtggcgcggacccgcgtgtttggccaactgtactgacattgtgacagtgtcgaactcatgtttatcctcttcgttcgttatctccaagtcatgcggtcgcgcagacctgggaaggccgtcgatctcgcggctgccgccaagctcgtcgctcccgtcacggtcaaataatcactattaccttgctttagccggggttgtttcattgtggtgactagtgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgggcgcgccgttt

SEQ ID NO: 42 - DNA, 13798 bp, Artificial Sequence
  Synthetic Polynucleotide
aaaccccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtatttaaatctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctggcgcgcccatttccacaactcatgccgagagaagtgtcagactgggcaactaaagtagtagtagtaatagctcgattaccatgatgaaatgctgggcgtcgaagcagctgcaggtccggcatgcagcaatccccactccgctccatcggctgctgttctggggtcaatccgtaccctcccaagttcacctcgccgctgacctcgggatcagctgcgttgtgcattcatgaataatgcgcaacatgagcaacccaacttcatcaagggagtttcgacgtcaccatatcaccacacatcttggaacagaattggggacaaggcagctggattgacggggaaatacataaaccggacgacctatgacgaccatcctatcccgtcaccgactccgatcccctgcggatgatggctcaaagaaccaagtttgatgagccggctgtgtgtcccagcacatacaacaaccgaagtatcagccccgttccgaacgcaggatcccagtctaccgaatcgattttggacagcccgagagaagccaaacaccgaaccgaaggaggaattgtcggaagacgtgcattatgagtcgttcacgcgcgcgttacatgttcgagcggaccttctggatcagctccctgcggaggatcttgcccgaggccgacttcgggacgctgttgatgaaggtgaccttgcggaggcgcttgaaggaggcgacctggccggcgatgaacttcttgacgtcgttctcggtcaggctggagttcggcgagcggacgacgtaggcgacggggacctcgccggcctcggcgtcggggaaggggatgacgacggcgtcgaggatctccgggtggctgaccaggaggccctccagctcggccggggcgacctggaagcccttgtacttgatgagctccttgatgcggtcgacgacgtacaggtggccgtcctcgtcgaagtagccgaggtcgccggtgtggacccagcccttcttgtcgatggtgagcttggtggcctgcgggttgttgaagtagccctgcatcatgttggggcccttgacccagatctcgccgagctggttcgggggcaggggcttcagggtgtcgaccgagacgatctgggcctcgacgcccgaggcgagcatgccggcggagccgctgttgcgcttgccgcccctgatgtcctccatgctgacgatgccgcacgtctcggtcatgccgtagccctgggcgacgatgccgtaggggacgaccttggagcactcctccatcaggtccttgccgagcggggcggcgccggagccgatgtacttgatcgagctgaggttgaacttcttgaccatgctgttcttggacagggcgaggatgacgggcgggacgacccagaggtgggtgaccttgtacttctcgacgtccttcagcatcttctcgaggtcgaagcgggccatcgagatgacggtgttgccgcgctgcagctgggcgtaggtgatgatggcgaggccgaagacgtggaacatcggcaggaagcagaggaagacgttgtccatctcgccgaccaggtcctgctccatggtgaccatgagggacgaggcgatgaagttcttgtgggtgaggacgacgcccttgctcatgccggtggtgcccgacgagtacaggagggcggcggtgtcggactgcttgaagtcgtcgacgatggggaactccgagccggagctgccgcccaggttgaccaggtcgttgaaggtcatgaccttgtcggaggagctctcctgctccgagtcggggccgatcaggatggtggggaggttgaagcccttgaccttctccaggagctgcgggacggtgatgatcagcttggggttggagtccttgacctgcttcgacagctcgctgacggtgtagagggggttggaggtggtggcgatggcgcccgaggcgatgatgccgaggaagcagaccgggaagtggatggagttgggggcgtagatcaggacgacgtcgttcttcttgatgcccaggttgaggaagccgtggctgaccttgatgacggtcgacttgaagtgggagaacgacaggatctggttcgtctccgagtcgatgagggccggcttctgggggtaggacgagctgttgcggaagaggaagctgaccatcgagaggttgttgttgttgggcaggtggaggggcgggcggaggctgcggtagatgccgtcgcggccgtagccggacttctccatgttgcttgcgtggtgtgctggaagctgagtgtattaggtggattgacaagtccctgcgggcaacgggaccgagtgagcaagccaggatcaggcgagcaagaggcaggtggtctgattctatcaacctacgtttagagacttgagatggaccagggaatgggcgttttgttttcgaattgatggtttacgatggatttcgttggacggaagaccgatgaggggaaaggagaggagaagcccaaagagggggtcggaggtggcctttattaagaggcggccggccgggcaatgggcagatcagccatctttgctgcatcgttcctgcgactgtttcgtcagaccgggcggggtaatgtcaggagagctccctggtaggttgcgggcagcggcagtgatcacgttgactggctcacgggatcgcgtgacgagtacatcatgatggcacgacctcgcaggcgagccctgcgtggcttaaacaggccaaggtacctggcccgagcgtcctgcagccagcgctaacagcccagcccaagccagaatctggggtaatctggggtaccggggtgcccgacccactgcgggcaaccagcgcttgtgcaccgcgtaaggcctcaacaagacatcagttagtatcgatgccgagattcagttggcaattacatacgtctaacttttccaatgcttattttgagtttcttgtagttatgcagctggtggaagttaggacaggaaccttgagtgacaagcaatccggccgggccgggaaggtgcccgctgcacgatcagtggggcaaaggtggggtatcccgagcaggagcgaactccaacagagtattcgatcaaaaaaggcaagtcctcccccaccatcctttgtagcttgcaatgcatctccttttttgcaatggattttgcttcgcgagtgctaatgccttgtgaaggactatgtggttggttcaaacctgttgttttgatccatctagtccacgttgcaggcatacaaatacccgacgacgtgtatcataagttaagtaggtactgtacgttagtttgtttaccttcttcagccagtagtccgactttgctctcaagtgctcatccaaccctttggccttccaaatcttgataccgagagcctcaggcaggttggtatattctacctcaaatacctgccagatgttgaccaatgccgttgtcgttactccgtactacgcgaaccaatgccaacatgattctcctttcagtcacgagacgattcgagtgtcatggtagcagtactttggtagtaaagagtcactcaattgaacatgtcgtagctcattcgtaaccaagtcatgataaccctgaatttcaggggttcccttcagagacaggagccgtcatgttgcggcaagagaaaaaaaaatgaagaggaaacacacaaaaacgtgggaagaatagccatcagcggctaccctcatactccaactcctgcctgccttaattaattacttgcgcggggtgtagtcgaagatcaggagcttctcccagaacgagcggtagacgtcgccgaagccgacgtgggccgggtggatgatgtagtcctggatcgtctcgacgctctcgaaggtgacctcgacgatgtgggtgtagccctcttccttgttcttctgggtgacgtccttgccccagtagacgtccttcatggcggggatgatgttgacgaggttgacgtaggtcttgaagaactcttccttctgggcctcggtgatctcgtccttgaacttcaggacgatgaggtgcttgacggccatggtgtgagggactaggtaggcttttgtagtgttgcagggagttggaggggatgtgcgacttctggtttgcttctcttaggttgagtatgaaagttgaggacgactcggggtcaagacgtcctgagagagagcgggagcatgctggcccccgacgccttcttaagcaaaccagctcatggcgcgtcagactggattcggaagatcgaccgggaacgagtaagggccagtggttggcacctctcgggcagcagcagcagcagcagcagcagcaatgtgccatggcatctgcgcgatcgggcatcgttgaccgctgttcccgcaggcgatgtaccatgttatccgcgcctgcctgcttgcgagtggtgccatggcaaatgctggaagcgggtccctcgctacagagtaaatccacggctgcaggagacgcgcagttggtcatccctggggcccctgcgccacgcggcactgccttacccctctgcacacgcgtgactaacccccactactgagtaccccgcttgtcaaaaggtcgcttccatacttatcgccagcctgacattatcgcgtctgcactggaaacctaagcgggtaaagcatcagagcatcaaatccaaggctctttctcctatctctgtaaatgagaggacaagttgatttcggaatcccgagtagaacggcagacagccaggcatactatcattacgcagctccggggaaagatccgacaaccagagccagtctctttctgccgttctgatgattccatcttcccctcagctccttcaccgcccagcgtctgctacgtgtccggcccggctttgcctgcctcgtcctgcagccaacgggactgcgcgaccgagccgccgactctgcaagtaatggtacctaacgaccgccccaagctggtagctctgtcctggtttcgccgcgtaagtctcggcgctagccttgattatgctgtctcggatctggaccaagtgtttcgatattccatcccatgacctacatgcgccggcgagacgcactacaagggcacgtgggatgccatttccaagatcgcatccgccgagggcgtgacgggtttgtacgccggcatgggcggctcgttgattggtgttgcctcgacgaatttcgcctacttttactggtactcggtcgtccgcactctctacttcaagtacgccaaggcgacaggccagccgtctaccgtcgtcgaactgtccctcggcgccgtcgccggcgcgcttgcgcagctcttcacgatccctgttgccgtgatcacgacgcggcaacaaacgcagagcaaggaagagaggaagggcatcatcgacactgcgcgcgaggtgattgagggtgaggacggcatctcggggctctggagggggttaaaggcgagcctggtcttggtggtcaacccctccatcacctatggtgcgtatgagaggttaaaggatgttctgttcccgggcaagaagaacctctctccttgggaagctttcggtaagccaggccattgctgtggatgcttgcagcgttcaggaagtattgacaggcttgtctagctcttggggccatgtccaaggcactcgccaccatcgtcacgcagcccctgatcgtggccaaggtcggcctgcaatcgaagccgccaccggcgcggcaggggaagccgttcaagagtttcgtcgaggtgatgcagttcatcatcgcgaacgagggccccctgagccttttcaagggcatcgggccacagattctcaagggcctgttggttcagggtattctgatgatgaccaaggagcggtatgtggcgcggacccgcgtgtttggccaactgtactgacattgtgacagtgtcgaactcatgtttatcctcttcgttcgttatctccaagtcatgcggtcgcgcagacctgggaaggccgtcgatctcgcggctgccgccaagctcgtcgctcccgtcacggtcaaataatcactattaccttgctttagccggggttgtttcattgtggtgactagtgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgggcgcgccgttt

SEQ ID NO: 43 - DNA, 12047 bp, Artificial Sequence
  Synthetic Polynucleotide
aaaccgtgtagacggtcgtgggcaggcgggcgaccaggccgatcgccgcggcgaagagcgggatggtcaggtaccactagttgtctgtactgtacaaacaatctggtcctgatatggtacggcagtctcccgtacttcgtattgcgtatgaacgagatgtacctacctggtatgtagggcatgaaatgtgtagcatctcgcgcatttaaccatatgtggtaatctgacgattgcttccactcgaggtcgaattgacctttcccagtttaaagatgtcgtgacaggggcggtaagtcgacgcagacagagctcaaactgggaaagccagaggaagggcggttcaagatagccagtaattccctggacactcaggaccgtcccgtcactctgactgagcgagcctgtcgatttgagacgatggggacgttgtctcgggctgggagtcccccatgaatcgagtcagtcttcagtcaatccagagaccgtttcggtggtaggttggaggaatgcccggggagtggaggctgtgcaggaaagaggtacatacctactatggagtagtatggagcttacacaacgagcggagccgcccccccagttgctctttcccgcacatgtgaaggtcatctccagaaaccaatcgaaggccaccaaagtattgaaagttgcaagctatttcccacccattacctttcaatcttttggggcctgctgcgagagagaaattcagtcaaagaaaagacgtccttcctctagtcgcttggtaagaaacgcaggctgtgggctgtgagagcgaggcacgcgttcacgcaagggcgggctggttgtatgcatgtatgtactgtacgtcttctgcagggcactatagacgggcaggtatgtactgtaccgtatgtcctgtacatacattcgcgcgggatgcgtaggggcctaactgtgtataccgtaccgcctactgaaaacccggaaaggcggggaatatccagatcttcgaaatacgcgaaacgccgaagccagcagcagcccgaagcatggaagactaccccaaagtaattgcgccagttcgccatgacccaaatcgcatgtcggctcggtcattgcacgtctttcaatattttgagggacagaaaagtcaccgacatcacaatcgttttgacatggtaggggaggtgctgaagatacggagtaccagtgacgtgaccgccatacccgctgaaaccccctcggtaccccctggcccgtgagcggggggccctgcaccgctcccgaagcctggaccgacgccggcgttaattaaggcaggcaggagttggagtatgagggtagccgctgatggctattcttcccacgtttttgtgtgtttcctcttcatttttttttctcttgccgcaacatgacggctcctgtctctgaagggaacccctgaaattcagggttatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtccgcgatgagcagagcattttaaaggaacgccgcactcacaaacaccaacactttagtgtctagtctacagaggcgtccctccccgtcttggatgcgtgattccattaccgtagatagtaccgcaaatgcacgggggtgtagtgtatgaaccacgctgggttcctgacctgacccggcaacccaatggagcagactcagggcccgctggccccggtggcgtatcaggtgactgttgggggagctaaccttggcaaacaaccgagctcagcgttaatgcatttcaagaagtcggtttgattgatcatccgcgaggaccgattatcgtacggcatcgaaaatcgtctcgccggagcgcacggattatttgaagaggctggcttgttgattgcaattgtcggctgccggccacgtcaccggccttgcagggcttatcagtaaatgcgggggcggagcagaggcggttcttgtcaagggtaggaggggtccggcaaagcccgagacggtggctgttcggaaacccaagaatggaccctgacagaacaattttcggattgggttcgttgcaaggatcgaacactacatcttccgagagagtttggaggttgtaagaacccttcgctaccgggagaacaaatcaccttgttgaatcagctctgtcactgctagtggcgagatggcctaagcagcgagactgttccccctgccccgctgtggatccgcatgactggtccattctggtcacttcgctccacttctctgcttttgcattgaccgctcagcggctgttgcgccttcctgacgcattcatagccccactcctgggcggcagcctggcgcttccaccatgcttgcccaacacgtatataaccttctcggcctaccctctaccacggagccactttctcttctccaacatcctccacacaacacccttctccttcgccatcaaagaggcatctatcggaaaatccaacatcgccagactcaccgaaacttcatacactcataacaactgcaaccatgaaccacctgcgcgccgagggcccggcctcggtcctcgccatcggcaccgccaaccccgagaacatcctcctgcaggacgagttcccggactactacttccgcgtcaccaagtccgagcacatgacccagctgaaggagaagttccgcaagatctgcgacaagagcatgatccgcaagcgcaactgcttcctcaacgaggagcacctgaagcagaacccgcgcctcgtcgagcacgagatgcagaccctggacgcccgccaggacatgctggtcgtcgaggtccccaagctgggcaaggacgcctgcgccaaggccatcaaggagtggggccagccgaagtcgaagatcacccacctgatcttcacctcggcctccaccaccgacatgccgggcgccgactaccactgcgccaagctgctgggcctctccccctcggtcaagcgcgtcatgatgtaccagctgggctgctacggtggcggcaccgtcctccgcatcgccaaggacatcgccgagaacaacaagggcgcccgcgtcctggccgtctgctgcgacatcatggcctgcctgttccgcggcccctccgagtcggacctggagctcctggtcggccaggccatcttcggcgacggcgccgccgccgtcatcgtcggcgccgagcccgacgagtcggtcggcgagcgcccgatcttcgagctggtcagcaccggccagaccatcctgcccaactcggagggcaccatcggcggccacatccgcgaggccggcctcatcttcgacctgcacaaggacgtcccgatgctgatctcgaacaacatcgagaagtgcctcatcgaggccttcacccccatcggcatcagcgactggaactcgatcttctggatcacccaccctggcggcaaggccatcctcgacaaggtcgaggagaagctccacctgaagtccgacaagttcgtcgactcccgccacgtcctgtcggagcacggcaacatgagctcgtccaccgtcctcttcgtcatggacgagctccgcaagcgctcgctggaggaaggcaagtcgaccaccggcgacggcttcgagtggggcgtcctgttcggcttcggcccgggcctcaccgtcgagcgcgtcgtcgtccgcagcgtcccgatcaagtactaacgcgcgcgagtgtctgcatcggacgggaatgggcctgggagcgttttagcgggtttgggacggccaaccattggctgccgctggaaatttggggtttaccattaatgacacggtaacatggagataccacggatgaatagactcgtttggagtcccccgattattgttcgtttgatgctgcgtaatcgtggtgcgatgacatttgatgcctatgggatggcgggggtctcccccgctttcggaagttgcatgtgaaaaacagttcctgctccgtcctagccttggcaatgcaaacttggatgttccggcttcgtaaccgcctttcacatccttcctccgacaatgcaggttgttgccgacaagccagcacgtcaatgatcctcatgatgcagcttgctgcaagagagcgcaagcttcgagaagcagagcattcattacctcccgtgcctccgtgaacacgtctcgtctcgtcggtcaaagttttgccaccatcatcctacactcggcgcgccctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcggtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggttt

SEQ ID NO: 44 - DNA, 14635 bp, Artificial Sequence
  Synthetic Polynucleotide
aaaccgtgtagacggtcgtgggcaggcgggcgaccaggccgatcgccgcggcgaagagcgggatggtcaggtaccactagttgtctgtactgtacaaacaatctggtcctgatatggtacggcagtctcccgtacttcgtattgcgtatgaacgagatgtacctacctggtatgtagggcatgaaatgtgtagcatctcgcgcatttaaccatatgtggtaatctgacgattgcttccactcgaggtcgaattgacctttcccagtttaaagatgtcgtgacaggggcggtaagtcgacgcagacagagctcaaactgggaaagccagaggaagggcggttcaagatagccagtaattccctggacactcaggaccgtcccgtcactctgactgagcgagcctgtcgatttgagacgatggggacgttgtctcgggctgggagtcccccatgaatcgagtcagtcttcagtcaatccagagaccgtttcggtggtaggttggaggaatgcccggggagtggaggctgtgcaggaaagaggtacatacctactatggagtagtatggagcttacacaacgagcggagccgcccccccagttgctctttcccgcacatgtgaaggtcatctccagaaaccaatcgaaggccaccaaagtattgaaagttgcaagctatttcccacccattacctttcaatcttttggggcctgctgcgagagagaaattcagtcaaagaaaagacgtccttcctctagtcgcttggtaagaaacgcaggctgtgggctgtgagagcgaggcacgcgttcacgcaagggcgggctggttgtatgcatgtatgtactgtacgtcttctgcagggcactatagacgggcaggtatgtactgtaccgtatgtcctgtacatacattcgcgcgggatgcgtaggggcctaactgtgtataccgtaccgcctactgaaaacccggaaaggcggggaatatccagatcttcgaaatacgcgaaacgccgaagccagcagcagcccgaagcatggaagactaccccaaagtaattgcgccagttcgccatgacccaaatcgcatgtcggctcggtcattgcacgtctttcaatattttgagggacagaaaagtcaccgacatcacaatcgttttgacatggtaggggaggtgctgaagatacggagtaccagtgacgtgaccgccatacccgctgaaaccccctcggtaccccctggcccgtgagcggggggccctgcaccgctcccgaagcctggaccgacgccggcggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgggcctcagcctcgtctgcaccttcagcttccagaccaactaccacacgctcctcaacccgcacaacaagaaccccaagaacagcctcctgtcctaccagcaccccaagaccccgatcatcaagagctcgtacgacaacttcccctccaagtactgcctgaccaagaacttccacctcctgggcctcaacagccacaaccgcatctcctcgcagagccgctccatccgcgccggctcggaccagatcgagggctcgccccaccacgagagcgacaactcgatcgccaccaagatcctgaacttcggccacacctgctggaagctccagcgcccgtacgtcgtcaagggcatgatctccatcgcctgcggcctgttcggccgcgagctcttcaacaaccgccacctgttctcgtggggcctcatgtggaaggccttcttcgccctggtcccgatcctctccttcaacttcttcgccgccatcatgaaccagatctacgacgtcgacatcgaccgcatcaacaagccggacctgccgctcgtctcgggcgagatgtccatcgagacggcctggatcctcagcatcatcgtcgccctgaccggcctcatcgtcaccatcaagctgaagtcggccccgctcttcgtcttcatctacatcttcggcatcttcgccggcttcgcctacagcgtcccgcccatccgctggaagcagtacccgttcaccaacttcctgatcaccatctcgtcccacgtcggcctcgccttcacctcctactcggccaccaccagcgccctgggcctccccttcgtctggcgcccggccttctcgttcatcatcgccttcatgaccgtcatgggcatgaccatcgccttcgccaaggacatctcggacatcgagggcgacgccaagtacggcgtctccaccgtcgccaccaagctgggcgcccgcaacatgaccttcgtcgtcagcggcgtcctcctgctcaactacctcgtctcgatctccatcggcatcatctggccccaggtcttcaagtccaacatcatgatcctcagccacgccatcctggccttctgcctcatcttccagacccgcgagctggccctcgccaactacgcctccgccccgagccgccagttcttcgagttcatctggctcctctactacgccgagtacttcgtctacgtcttcatctgattaattaaggcaggcaggagttggagtatgagggtagccgctgatggctattcttcccacgtttttgtgtgtttcctcttcatttttttttctcttgccgcaacatgacggctcctgtctctgaagggaacccctgaaattcagggttatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtccgcgatgagcagagcattttaaaggaacgccgcactcacaaacaccaacactttagtgtctagtctacagaggcgtccctccccgtcttggatgcgtgattccattaccgtagatagtaccgcaaatgcacgggggtgtagtgtatgaaccacgctgggttcctgacctgacccggcaacccaatggagcagactcagggcccgctggccccggtggcgtatcaggtgactgttgggggagctaaccttggcaaacaaccgagctcagcgttaatgcatttcaagaagtcggtttgattgatcatccgcgaggaccgattatcgtacggcatcgaaaatcgtctcgccggagcgcacggattatttgaagaggctggcttgttgattgcaattgtcggctgccggccacgtcaccggccttgcagggcttatcagtaaatgcgggggcggagcagaggcggttcttgtcaagggtaggaggggtccggcaaagcccgagacggtggctgttcggaaacccaagaatggaccctgacagaacaattttcggattgggttcgttgcaaggatcgaacactacatcttccgagagagtttggaggttgtaagaacccttcgctaccgggagaacaaatcaccttgttgaatcagctctgtcactgctagtggcgagatggcctaagcagcgagactgttccccctgccccgctgtggatccgcatgactggtccattctggtcacttcgctccacttctctgcttttgcattgaccgctcagcggctgttgcgccttcctgacgcattcatagccccactcctgggcggcagcctggcgcttccaccatgcttgcccaacacgtatataaccttctcggcctaccctctaccacggagccactttctcttctccaacatcctccacacaacacccttctccttcgccatcaaagaggcatctatcggaaaatccaacatcgccagactcaccgaaacttcatacactcataacaactgcaaccatgaaccacctgcgcgccgagggcccggcctcggtcctcgccatcggcaccgccaaccccgagaacatcctcctgcaggacgagttcccggactactacttccgcgtcaccaagtccgagcacatgacccagctgaaggagaagttccgcaagatctgcgacaagagcatgatccgcaagcgcaactgcttcctcaacgaggagcacctgaagcagaacccgcgcctcgtcgagcacgagatgcagaccctggacgcccgccaggacatgctggtcgtcgaggtccccaagctgggcaaggacgcctgcgccaaggccatcaaggagtggggccagccgaagtcgaagatcacccacctgatcttcacctcggcctccaccaccgacatgccgggcgccgactaccactgcgccaagctgctgggcctctccccctcggtcaagcgcgtcatgatgtaccagctgggctgctacggtggcggcaccgtcctccgcatcgccaaggacatcgccgagaacaacaagggcgcccgcgtcctggccgtctgctgcgacatcatggcctgcctgttccgcggcccctccgagtcggacctggagctcctggtcggccaggccatcttcggcgacggcgccgccgccgtcatcgtcggcgccgagcccgacgagtcggtcggcgagcgcccgatcttcgagctggtcagcaccggccagaccatcctgcccaactcggagggcaccatcggcggccacatccgcgaggccggcctcatcttcgacctgcacaaggacgtcccgatgctgatctcgaacaacatcgagaagtgcctcatcgaggccttcacccccatcggcatcagcgactggaactcgatcttctggatcacccaccctggcggcaaggccatcctcgacaaggtcgaggagaagctccacctgaagtccgacaagttcgtcgactcccgccacgtcctgtcggagcacggcaacatgagctcgtccaccgtcctcttcgtcatggacgagctccgcaagcgctcgctggaggaaggcaagtcgaccaccggcgacggcttcgagtggggcgtcctgttcggcttcggcccgggcctcaccgtcgagcgcgtcgtcgtccgcagcgtcccgatcaagtactaacgcgcgcgagtgtctgcatcggacgggaatgggcctgggagcgttttagcgggtttgggacggccaaccattggctgccgctggaaatttggggtttaccattaatgacacggtaacatggagataccacggatgaatagactcgtttggagtcccccgattattgttcgtttgatgctgcgtaatcgtggtgcgatgacatttgatgcctatgggatggcgggggtctcccccgctttcggaagttgcatgtgaaaaacagttcctgctccgtcctagccttggcaatgcaaacttggatgttccggcttcgtaaccgcctttcacatccttcctccgacaatgcaggttgttgccgacaagccagcacgtcaatgatcctcatgatgcagcttgctgcaagagagcgcaagcttcgagaagcagagcattcattacctcccgtgcctccgtgaacacgtctcgtctcgtcggtcaaagttttgccaccatcatcctacactcggcgcgccctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcggtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggttt

SEQ ID NO: 45 - DNA, 14626 bp, Artificial Sequence
  Synthetic Polynucleotide
aaaccgtgtagacggtcgtgggcaggcgggcgaccaggccgatcgccgcggcgaagagcgggatggtcaggtaccactagttgtctgtactgtacaaacaatctggtcctgatatggtacggcagtctcccgtacttcgtattgcgtatgaacgagatgtacctacctggtatgtagggcatgaaatgtgtagcatctcgcgcatttaaccatatgtggtaatctgacgattgcttccactcgaggtcgaattgacctttcccagtttaaagatgtcgtgacaggggcggtaagtcgacgcagacagagctcaaactgggaaagccagaggaagggcggttcaagatagccagtaattccctggacactcaggaccgtcccgtcactctgactgagcgagcctgtcgatttgagacgatggggacgttgtctcgggctgggagtcccccatgaatcgagtcagtcttcagtcaatccagagaccgtttcggtggtaggttggaggaatgcccggggagtggaggctgtgcaggaaagaggtacatacctactatggagtagtatggagcttacacaacgagcggagccgcccccccagttgctctttcccgcacatgtgaaggtcatctccagaaaccaatcgaaggccaccaaagtattgaaagttgcaagctatttcccacccattacctttcaatcttttggggcctgctgcgagagagaaattcagtcaaagaaaagacgtccttcctctagtcgcttggtaagaaacgcaggctgtgggctgtgagagcgaggcacgcgttcacgcaagggcgggctggttgtatgcatgtatgtactgtacgtcttctgcagggcactatagacgggcaggtatgtactgtaccgtatgtcctgtacatacattcgcgcgggatgcgtaggggcctaactgtgtataccgtaccgcctactgaaaacccggaaaggcggggaatatccagatcttcgaaatacgcgaaacgccgaagccagcagcagcccgaagcatggaagactaccccaaagtaattgcgccagttcgccatgacccaaatcgcatgtcggctcggtcattgcacgtctttcaatattttgagggacagaaaagtcaccgacatcacaatcgttttgacatggtaggggaggtgctgaagatacggagtaccagtgacgtgaccgccatacccgctgaaaccccctcggtaccccctggcccgtgagcggggggccctgcaccgctcccgaagcctggaccgacgccggcggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgggcctcagctcggtctgcaccttctcgttccagaccaactaccacaccctcctgaacccccacaacaacaacccgaagacctccctcctgtgctaccgccaccccaagaccccgatcaagtacagctacaacaacttcccctcgaagcactgctcgaccaagtccttccacctccagaacaagtgctccgagagcctgtcgatcgccaagaactcgatccgcgccgccaccaccaaccagaccgagcctcccgagtccgacaaccacagcgtcgccaccaagatcctcaacttcggcaaggcctgctggaagctgcagcgcccgtacaccatcatcgccttcacctcctgcgcctgcggcctcttcggcaaggagctcctgcacaacaccaacctcatctcctggagcctgatgttcaaggccttcttcttcctcgtcgccatcctgtgcatcgcctcgttcaccacgaccatcaaccagatctacgacctccacatcgaccgcatcaacaagccggacctccccctggcctccggcgagatctccgtcaacaccgcctggatcatgtccatcatcgtcgccctcttcggcctgatcatcaccatcaagatgaagggcggccccctctacatcttcggctactgcttcggcatcttcggtggcatcgtctacagcgtcccgcccttccgctggaagcagaacccgtcgaccgccttcctcctgaacttcctcgcccacatcatcaccaacttcaccttctactacgcctcccgcgccgccctgggcctgcccttcgagctgcgcccgagcttcaccttcctcctggccttcatgaagagcatgggctccgccctggccctgatcaaggacgccagcgacgtcgagggcgacaccaagttcggcatcagcaccctcgcctcgaagtacggctcccgcaacctcaccctgttctgctccggcatcgtcctgctcagctacgtcgccgccatcctggccggcatcatctggccccaggccttcaactcgaacgtcatgctcctgtcccacgccatcctcgccttctggctcatcctgcagacccgcgacttcgccctgaccaactacgaccccgaggccggccgcaggttctacgagttcatgtggaagctctactacgccgagtacctggtctacgtcttcatctaattaattaaggcaggcaggagttggagtatgagggtagccgctgatggctattcttcccacgtttttgtgtgtttcctcttcatttttttttctcttgccgcaacatgacggctcctgtctctgaagggaacccctgaaattcagggttatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtccgcgatgagcagagcattttaaaggaacgccgcactcacaaacaccaacactttagtgtctagtctacagaggcgtccctccccgtcttggatgcgtgattccattaccgtagatagtaccgcaaatgcacgggggtgtagtgtatgaaccacgctgggttcctgacctgacccggcaacccaatggagcagactcagggcccgctggccccggtggcgtatcaggtgactgttgggggagctaaccttggcaaacaaccgagctcagcgttaatgcatttcaagaagtcggtttgattgatcatccgcgaggaccgattatcgtacggcatcgaaaatcgtctcgccggagcgcacggattatttgaagaggctggcttgttgattgcaattgtcggctgccggccacgtcaccggccttgcagggcttatcagtaaatgcgggggcggagcagaggcggttcttgtcaagggtaggaggggtccggcaaagcccgagacggtggctgttcggaaacccaagaatggaccctgacagaacaattttcggattgggttcgttgcaaggatcgaacactacatcttccgagagagtttggaggttgtaagaacccttcgctaccgggagaacaaatcaccttgttgaatcagctctgtcactgctagtggcgagatggcctaagcagcgagactgttccccctgccccgctgtggatccgcatgactggtccattctggtcacttcgctccacttctctgcttttgcattgaccgctcagcggctgttgcgccttcctgacgcattcatagccccactcctgggcggcagcctggcgcttccaccatgcttgcccaacacgtatataaccttctcggcctaccctctaccacggagccactttctcttctccaacatcctccacacaacacccttctccttcgccatcaaagaggcatctatcggaaaatccaacatcgccagactcaccgaaacttcatacactcataacaactgcaaccatgaaccacctgcgcgccgagggcccggcctcggtcctcgccatcggcaccgccaaccccgagaacatcctcctgcaggacgagttcccggactactacttccgcgtcaccaagtccgagcacatgacccagctgaaggagaagttccgcaagatctgcgacaagagcatgatccgcaagcgcaactgcttcctcaacgaggagcacctgaagcagaacccgcgcctcgtcgagcacgagatgcagaccctggacgcccgccaggacatgctggtcgtcgaggtccccaagctgggcaaggacgcctgcgccaaggccatcaaggagtggggccagccgaagtcgaagatcacccacctgatcttcacctcggcctccaccaccgacatgccgggcgccgactaccactgcgccaagctgctgggcctctccccctcggtcaagcgcgtcatgatgtaccagctgggctgctacggtggcggcaccgtcctccgcatcgccaaggacatcgccgagaacaacaagggcgcccgcgtcctggccgtctgctgcgacatcatggcctgcctgttccgcggcccctccgagtcggacctggagctcctggtcggccaggccatcttcggcgacggcgccgccgccgtcatcgtcggcgccgagcccgacgagtcggtcggcgagcgcccgatcttcgagctggtcagcaccggccagaccatcctgcccaactcggagggcaccatcggcggccacatccgcgaggccggcctcatcttcgacctgcacaaggacgtcccgatgctgatctcgaacaacatcgagaagtgcctcatcgaggccttcacccccatcggcatcagcgactggaactcgatcttctggatcacccaccctggcggcaaggccatcctcgacaaggtcgaggagaagctccacctgaagtccgacaagttcgtcgactcccgccacgtcctgtcggagcacggcaacatgagctcgtccaccgtcctcttcgtcatggacgagctccgcaagcgctcgctggaggaaggcaagtcgaccaccggcgacggcttcgagtggggcgtcctgttcggcttcggcccgggcctcaccgtcgagcgcgtcgtcgtccgcagcgtcccgatcaagtactaacgcgcgcgagtgtctgcatcggacgggaatgggcctgggagcgttttagcgggtttgggacggccaaccattggctgccgctggaaatttggggtttaccattaatgacacggtaacatggagataccacggatgaatagactcgtttggagtcccccgattattgttcgtttgatgctgcgtaatcgtggtgcgatgacatttgatgcctatgggatggcgggggtctcccccgctttcggaagttgcatgtgaaaaacagttcctgctccgtcctagccttggcaatgcaaacttggatgttccggcttcgtaaccgcctttcacatccttcctccgacaatgcaggttgttgccgacaagccagcacgtcaatgatcctcatgatgcagcttgctgcaagagagcgcaagcttcgagaagcagagcattcattacctcccgtgcctccgtgaacacgtctcgtctcgtcggtcaaagttttgccaccatcatcctacactcggcgcgccctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcggtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggttt

SEQ ID NO: 46 - DNA, 14362 bp, Artificial Sequence
  Synthetic Polynucleotide
aaaccgtgtagacggtcgtgggcaggcgggcgaccaggccgatcgccgcggcgaagagcgggatggtcaggtaccactagttgtctgtactgtacaaacaatctggtcctgatatggtacggcagtctcccgtacttcgtattgcgtatgaacgagatgtacctacctggtatgtagggcatgaaatgtgtagcatctcgcgcatttaaccatatgtggtaatctgacgattgcttccactcgaggtcgaattgacctttcccagtttaaagatgtcgtgacaggggcggtaagtcgacgcagacagagctcaaactgggaaagccagaggaagggcggttcaagatagccagtaattccctggacactcaggaccgtcccgtcactctgactgagcgagcctgtcgatttgagacgatggggacgttgtctcgggctgggagtcccccatgaatcgagtcagtcttcagtcaatccagagaccgtttcggtggtaggttggaggaatgcccggggagtggaggctgtgcaggaaagaggtacatacctactatggagtagtatggagcttacacaacgagcggagccgcccccccagttgctctttcccgcacatgtgaaggtcatctccagaaaccaatcgaaggccaccaaagtattgaaagttgcaagctatttcccacccattacctttcaatcttttggggcctgctgcgagagagaaattcagtcaaagaaaagacgtccttcctctagtcgcttggtaagaaacgcaggctgtgggctgtgagagcgaggcacgcgttcacgcaagggcgggctggttgtatgcatgtatgtactgtacgtcttctgcagggcactatagacgggcaggtatgtactgtaccgtatgtcctgtacatacattcgcgcgggatgcgtaggggcctaactgtgtataccgtaccgcctactgaaaacccggaaaggcggggaatatccagatcttcgaaatacgcgaaacgccgaagccagcagcagcccgaagcatggaagactaccccaaagtaattgcgccagttcgccatgacccaaatcgcatgtcggctcggtcattgcacgtctttcaatattttgagggacagaaaagtcaccgacatcacaatcgttttgacatggtaggggaggtgctgaagatacggagtaccagtgacgtgaccgccatacccgctgaaaccccctcggtaccccctggcccgtgagcggggggccctgcaccgctcccgaagcctggaccgacgccggcggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtccgaggccgccgacgtcgagcgcgtctacgccgccatggaggaagccgccggcctcctcggcgtcgcctgcgcccgcgacaagatctaccccctcctgagcaccttccaggacaccctggtcgagggcggctccgtcgtcgtcttcagcatggcctccggccgccactccaccgagctcgacttctccatctcggtccccaccagccacggcgacccgtacgccaccgtcgtcgagaagggcctgttcccggccaccggccaccccgtcgacgacctcctggccgacacccagaagcacctccccgtcagcatgttcgccatcgacggcgaggtcaccggcggcttcaagaagacctacgccttcttcccgaccgacaacatgcccggcgtcgccgagctctccgccatcccctccatgcctcccgccgtcgccgagaacgccgagctgttcgcccgctacggcctcgacaaggtccagatgacctcgatggactacaagaagcgccaggtcaacctctacttctcggagctgtcggcccagaccctggaggccgagtcggtcctggccctggtccgcgagctgggcctgcacgtccccaacgagctcggcctgaagttctgcaagcgctcgttctccgtctacccgaccctgaactgggagacgggcaagatcgaccgcctctgcttcgccgtcatctccaacgacccgaccctggtccccagctccgacgagggcgacatcgagaagttccacaactacgccaccaaggccccctacgcctacgtcggcgagaagcgcaccctggtctacggcctcaccctgagcccgaaggaagagtactacaagctcggcgcctactaccacatcaccgacgtccagcgcggcctcctgaaggccttcgactcgctcgaggactaattaattaaggcaggcaggagttggagtatgagggtagccgctgatggctattcttcccacgtttttgtgtgtttcctcttcatttttttttctcttgccgcaacatgacggctcctgtctctgaagggaacccctgaaattcagggttatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtccgcgatgagcagagcattttaaaggaacgccgcactcacaaacaccaacactttagtgtctagtctacagaggcgtccctccccgtcttggatgcgtgattccattaccgtagatagtaccgcaaatgcacgggggtgtagtgtatgaaccacgctgggttcctgacctgacccggcaacccaatggagcagactcagggcccgctggccccggtggcgtatcaggtgactgttgggggagctaaccttggcaaacaaccgagctcagcgttaatgcatttcaagaagtcggtttgattgatcatccgcgaggaccgattatcgtacggcatcgaaaatcgtctcgccggagcgcacggattatttgaagaggctggcttgttgattgcaattgtcggctgccggccacgtcaccggccttgcagggcttatcagtaaatgcgggggcggagcagaggcggttcttgtcaagggtaggaggggtccggcaaagcccgagacggtggctgttcggaaacccaagaatggaccctgacagaacaattttcggattgggttcgttgcaaggatcgaacactacatcttccgagagagtttggaggttgtaagaacccttcgctaccgggagaacaaatcaccttgttgaatcagctctgtcactgctagtggcgagatggcctaagcagcgagactgttccccctgccccgctgtggatccgcatgactggtccattctggtcacttcgctccacttctctgcttttgcattgaccgctcagcggctgttgcgccttcctgacgcattcatagccccactcctgggcggcagcctggcgcttccaccatgcttgcccaacacgtatataaccttctcggcctaccctctaccacggagccactttctcttctccaacatcctccacacaacacccttctccttcgccatcaaagaggcatctatcggaaaatccaacatcgccagactcaccgaaacttcatacactcataacaactgcaaccatgaaccacctgcgcgccgagggcccggcctcggtcctcgccatcggcaccgccaaccccgagaacatcctcctgcaggacgagttcccggactactacttccgcgtcaccaagtccgagcacatgacccagctgaaggagaagttccgcaagatctgcgacaagagcatgatccgcaagcgcaactgcttcctcaacgaggagcacctgaagcagaacccgcgcctcgtcgagcacgagatgcagaccctggacgcccgccaggacatgctggtcgtcgaggtccccaagctgggcaaggacgcctgcgccaaggccatcaaggagtggggccagccgaagtcgaagatcacccacctgatcttcacctcggcctccaccaccgacatgccgggcgccgactaccactgcgccaagctgctgggcctctccccctcggtcaagcgcgtcatgatgtaccagctgggctgctacggtggcggcaccgtcctccgcatcgccaaggacatcgccgagaacaacaagggcgcccgcgtcctggccgtctgctgcgacatcatggcctgcctgttccgcggcccctccgagtcggacctggagctcctggtcggccaggccatcttcggcgacggcgccgccgccgtcatcgtcggcgccgagcccgacgagtcggtcggcgagcgcccgatcttcgagctggtcagcaccggccagaccatcctgcccaactcggagggcaccatcggcggccacatccgcgaggccggcctcatcttcgacctgcacaaggacgtcccgatgctgatctcgaacaacatcgagaagtgcctcatcgaggccttcacccccatcggcatcagcgactggaactcgatcttctggatcacccaccctggcggcaaggccatcctcgacaaggtcgaggagaagctccacctgaagtccgacaagttcgtcgactcccgccacgtcctgtcggagcacggcaacatgagctcgtccaccgtcctcttcgtcatggacgagctccgcaagcgctcgctggaggaaggcaagtcgaccaccggcgacggcttcgagtggggcgtcctgttcggcttcggcccgggcctcaccgtcgagcgcgtcgtcgtccgcagcgtcccgatcaagtactaacgcgcgcgagtgtctgcatcggacgggaatgggcctgggagcgttttagcgggtttgggacggccaaccattggctgccgctggaaatttggggtttaccattaatgacacggtaacatggagataccacggatgaatagactcgtttggagtcccccgattattgttcgtttgatgctgcgtaatcgtggtgcgatgacatttgatgcctatgggatggcgggggtctcccccgctttcggaagttgcatgtgaaaaacagttcctgctccgtcctagccttggcaatgcaaacttggatgttccggcttcgtaaccgcctttcacatccttcctccgacaatgcaggttgttgccgacaagccagcacgtcaatgatcctcatgatgcagcttgctgcaagagagcgcaagcttcgagaagcagagcattcattacctcccgtgcctccgtgaacacgtctcgtctcgtcggtcaaagttttgccaccatcatcctacactcggcgcgccctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcggtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggttt

SEQ ID NO: 47 - DNA, 14839 bp, Artificial Sequence
  Synthetic Polynucleotide
gtttaaaccgtcagacgaggggaaactgcctacccctgttcaccaccgacatgggcagactgtgtgccgagaagagcagcaccaccttgttcctcctctcctccggatactccagcaacttggcctcgatgttctgcgcaaacgcctcgacgaggcccgggtgtgtcggccaccggtcgatcacgctccacttgatcgtcccgtccgtcccgtcgttccgcagcgcggccttgccctccaacctctggcgccacttccacagctcgtttaggctactccccgtcgtcgagcacgagtactgcgggtactgggtgaatgcgacggcgcggcccccgcggccgttcccgaacccgtcggccaacatctgccggtacatgtcctcggtgagcgggttcgcgtagcggaaggcgacatagggcatatgcggtgccgtctcgggcgagatcttatcgaggattttgcacatctcggcgcactggtgctcggaccacttgcggatgggtgagccgccgccgatggccgcatattgttgctggatcttgggcgtgcgccgcttggagaggagcgggccgatgtagccctggagccggccgagagggatgagatcgccatcggactggaggtgaaacaaagcgttaggcgatcggtccgggcggccgtattttggagcaccggggggggggggggttaactcacgaatagtctgctgaggaagtcgcccacttcatcggtcgtcgatgggccgcccatgttgagaaacaccatagccgttgggccccgcccagagtcttgggtgactggatgaaccggcgtcgcgagccatcgtgcctgttgtgcagaaggttttgccagcctatgaggccggagtggccgcatggcggccgggagacgaagcgccatctcgaagcggaacacgggaatccgaggcgagttcgcagtaaaaaaagaaaaaaaaaatgaaaaagaagcgctgttagtcgttgcagtaaaaaagataaacaagaacaaacgggattgagacaatccctagggccatctatcaatttattcgcaatgcgtcagaggaaactgacgataccttggtttcagacagtggcgaacggaacaggaggccagatcacactccgcccgcgactttcgcggcaactcggcggcggtacgatcaaaggccgactttgccatcttggcatcggcgttgaccttgcagatcggccgggatcccttttggccaatcgcaaatgttcaattgcacagcttgccttgtggcgcgccctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtatttaaatctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctgcggccgcgtcgggtatttgtatgcctgcaacgtggactagatggatcaaaacaacaggtttgaaccaaccacatagtccttcacaaggcattagcactcgcgaagcaaaatccattgcaaaaaaggagatgcattgcaagctacaaaggatggtgggggaggacttgccttttttgatcgaatactctgttggagttcgctcctgctcgggataccccacctttgccccactgatcgtgcagcgggcaccttcccggcccggccggattgcttgtcactcaaggttcctgtcctaacttccaccagctgcataactacaagaaactcaaaataagcattggaaaagttagacgtatgtaattgccaactgaatctcggcatcgatactaactgatgtcttgttgaggccttacgcggtgcacaagcgctggttgcccgcagtgggtcgggcaccccggtaccccagattaccccagattctggcttgggctgggctgttagcgctggctgcaggacgctcgggccaggtaccttggcctgtttaagccacgcagggctcgcctgcgaggtcgtgccatcatgatgtactcgtcacgcgatcccgtgagccagtcaacgtgatcactgccgctgcccgcaacctaccagggagctctcctgacattaccccgcccggtctgacgaaacagtcgcaggaacgatgcagcaaagatggctgatctgcccattgcccggccggccgcctcttaataaaggccacctccgaccccctctttgggcttctcctctcctttcccctcatcggtcttccgtccaacgaaatccatcgtaaaccatcaattcgaaaacaaaacgcccattccctggtccatctcaagtctctaaacgtaggttgatagaatcagaccacctgcctcttgctcgcctgatcctggcttgctcactcggtcccgttgcccgcagggacttgtcaatccacctaatacactcagcttccagcacaccacgcaagcaacatgggcaagaactacaagagcctggactcggtcgtcgcctccgacttcatcgccctgggcatcacctcggaggtcgccgagacgctccacggccgcctggccgagatcgtctgcaactacggcgccgccaccccgcagacctggatcaacatcgccaaccacatcctctcgcccgacctgccgttctccctccaccagatgctgttctacggctgctacaaggacttcggccccgcccctcccgcctggatcccggaccccgagaaggtcaagtccaccaacctgggcgccctcctggagaagcgcggcaaggagttcctcggcgtcaagtacaaggaccccatcagctcgttcagccacttccaggagttctcggtccgcaacccggaggtctactggcgcaccgtcctgatggacgagatgaagatcagcttctcgaaggaccccgagtgcatcctccgcagggacgacatcaacaaccctggcggctcggagtggctccctggcggctacctgaactcggccaagaactgcctgaacgtcaactccaacaagaagctcaacgacaccatgatcgtctggcgcgacgagggcaacgacgacctccccctgaacaagctcaccctcgaccagctgcgcaagcgcgtctggctggtcggctacgccctggaggagatgggcctcgagaagggctgcgccatcgccatcgacatgccgatgcacgtcgacgccgtcgtcatctacctcgccatcgtcctggccggctacgtcgtcgtcagcatcgccgactcgttctcggccccggagatctccacccgcctccgcctgagcaaggccaaggccatcttcacccaggaccacatcatccgcggcaagaagcgcatcccgctgtactcgcgcgtcgtcgaggccaagtcccccatggccatcgtcatcccctgctccggctcgaacatcggcgccgagctgcgcgacggcgacatcagctgggactacttcctcgagcgcgccaaggagttcaagaactgcgagttcaccgcccgcgagcagccggtcgacgcctacaccaacatcctcttctcctcgggcaccaccggcgagcccaaggccatcccctggacccaggccaccccgctgaaggccgccgccgacggctggtcgcacctcgacatccgcaagggcgacgtcatcgtctggcccaccaacctgggctggatgatgggcccctggctggtctacgcctccctcctgaacggcgcctccatcgccctgtacaacggcagccccctcgtctcgggcttcgccaagttcgtccaggacgccaaggtcaccatgctgggcgtcgtcccctccatcgtccgctcctggaagagcaccaactgcgtctccggctacgactggagcaccatccgctgcttctcctcgtcgggcgaggccagcaacgtcgacgagtacctctggctgatgggccgcgccaactacaagcccgtcatcgagatgtgcggtggcaccgagatcggcggcgccttctccgccggctccttcctgcaggcccagtccctctcgtccttcagctcgcagtgcatgggctgcaccctctacatcctggacaagaacggctaccccatgccgaagaacaagccgggcatcggcgagctggccctgggcccggtcatgttcggcgcctcgaagaccctcctgaacggcaaccaccacgacgtctacttcaagggcatgcccaccctcaacggcgaggtcctccgcaggcacggcgacatcttcgagctcacctccaacggctactaccacgcccacggccgcgccgacgacaccatgaacatcggcggcatcaagatctccagcatcgagatcgagcgcgtctgcaacgaggtcgacgaccgcgtcttcgagacgaccgccatcggcgtcccgcccctcggcggcggccccgagcagctggtcatcttcttcgtcctcaaggacagcaacgacaccaccatcgacctcaaccagctccgcctgtcgttcaacctcggcctgcagaagaagctcaacccgctgttcaaggtcacccgcgtcgtccccctctcctccctgccccgcaccgccaccaacaagatcatgcgcagggtcctccgccagcagttcagccacttcgagtaacgcgcgcgtgaacgactcataatgcacgtcttccgacaattcctccttcggttcggtgtttggcttctctcgggctgtccaaaatcgattcggtagactgggatcctgcgttcggaacggggctgatacttcggttgttgtatgtgctgggacacacagccggctcatcaaacttggttctttgagccatcatccgcaggggatcggagtcggtgacgggataggatggtcgtcataggtcgtccggtttatgtatttccccgtcaatccagctgccttgtccccaattctgttccaagatgtgtggtgatatggtgacgtcgaaactcccttgatgaagttgggttgctcatgttgcgcattattcatgaatgcacaacgcagctgatcccgaggtcagcggcgaggtgaacttgggagggtacggattgaccccagaacagcagccgatggagcggagtggggattgctgcatgccggacctgcagctgcttcgacgcccagcatttcatcatggtaatcgagctattactactactactttagttgcccagtctgacacttctctcggcatgagttgtggaaatggaattcccgagatgctctggagctgaagttccaaggcgtttttggagagagattgcggaactccaaacataaggtagagagagatattcctcagtccgcactaaacaaggtccctgtttaatagttacacagcaatggagatccatgcactcccgcacgtctggatgcacccacccttgctgctctctcggccccgctttggtctccttccactcattgccagttctgactggttcgcaacaacgcatgtcctcgtacgtccgcacgcagccactccactttacaatagaaactaaagatacccgcttggcaaagcgacacgacgacgcgacggagatactggtggtttgtcgcgccgtcctgttttctgatccaaacgacagccttgtcatggagactctgacctctgcattctgaagccaagcgaatgagcgcaggcgacccgacctacttgaaagagaacgagcggcaatggaggctctgctgggcaccggccagtcgaacccgacctgcggttcgctggccgacctccaggagcaactccggcatcttcttcagagtcgcgtgaccgaaactcgcgccgaacatatttcggtggcattcgaagtccgagcgaccgcgatttttgacatccctgtgaccggcgtcgaaaatgacctgctcgggaacccctcgaacatcgacccgtcgctgggcgggtcacggtcgagcgctgctgcgccagccatcaacggtagtgcgggacagccgacccgacgagtcagcgccatcgacgccctgatcaaccagcccgtggacgacccggtgttgcagactgcgattgccaggcagatcatatcgtcggtgggcgaggccgactcgagcaactgggcagtgcggcaggtctcgcgcgctgagcagagttggacgtttgcctacatctgcaaggattcctgggaggcctggaaccgtcaggcgtcgaagacacccgcgaagacgctcatcggggagtggagcggggagggtgggcaagatcccgttcatatgggtaggttcgttccacgctaccaagggtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 48 - DNA, 14410 bp, Artificial Sequence
  Synthetic Polynucleotide
aaaccgtgtagacggtcgtgggcaggcgggcgaccaggccgatcgccgcggcgaagagcgggatggtcaggtaccactagttgtctgtactgtacaaacaatctggtcctgatatggtacggcagtctcccgtacttcgtattgcgtatgaacgagatgtacctacctggtatgtagggcatgaaatgtgtagcatctcgcgcatttaaccatatgtggtaatctgacgattgcttccactcgaggtcgaattgacctttcccagtttaaagatgtcgtgacaggggcggtaagtcgacgcagacagagctcaaactgggaaagccagaggaagggcggttcaagatagccagtaattccctggacactcaggaccgtcccgtcactctgactgagcgagcctgtcgatttgagacgatggggacgttgtctcgggctgggagtcccccatgaatcgagtcagtcttcagtcaatccagagaccgtttcggtggtaggttggaggaatgcccggggagtggaggctgtgcaggaaagaggtacatacctactatggagtagtatggagcttacacaacgagcggagccgcccccccagttgctctttcccgcacatgtgaaggtcatctccagaaaccaatcgaaggccaccaaagtattgaaagttgcaagctatttcccacccattacctttcaatcttttggggcctgctgcgagagagaaattcagtcaaagaaaagacgtccttcctctagtcgcttggtaagaaacgcaggctgtgggctgtgagagcgaggcacgcgttcacgcaagggcgggctggttgtatgcatgtatgtactgtacgtcttctgcagggcactatagacgggcaggtatgtactgtaccgtatgtcctgtacatacattcgcgcgggatgcgtaggggcctaactgtgtataccgtaccgcctactgaaaacccggaaaggcggggaatatccagatcttcgaaatacgcgaaacgccgaagccagcagcagcccgaagcatggaagactaccccaaagtaattgcgccagttcgccatgacccaaatcgcatgtcggctcggtcattgcacgtctttcaatattttgagggacagaaaagtcaccgacatcacaatcgttttgacatggtaggggaggtgctgaagatacggagtaccagtgacgtgaccgccatacccgctgaaaccccctcggtaccccctggcccgtgagcggggggccctgcaccgctcccgaagcctggaccgacgccggcggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtcggccggctcggaccagatcgagggctcgccccaccacgagagcgacaactcgatcgccaccaagatcctgaacttcggccacacctgctggaagctccagcgcccgtacgtcgtcaagggcatgatctccatcgcctgcggcctgttcggccgcgagctcttcaacaaccgccacctgttctcgtggggcctcatgtggaaggccttcttcgccctggtcccgatcctctccttcaacttcttcgccgccatcatgaaccagatctacgacgtcgacatcgaccgcatcaacaagccggacctgccgctcgtctcgggcgagatgtccatcgagacggcctggatcctcagcatcatcgtcgccctgaccggcctcatcgtcaccatcaagctgaagtcggccccgctcttcgtcttcatctacatcttcggcatcttcgccggcttcgcctacagcgtcccgcccatccgctggaagcagtacccgttcaccaacttcctgatcaccatctcgtcccacgtcggcctcgccttcacctcctactcggccaccaccagcgccctgggcctccccttcgtctggcgcccggccttctcgttcatcatcgccttcatgaccgtcatgggcatgaccatcgccttcgccaaggacatctcggacatcgagggcgacgccaagtacggcgtctccaccgtcgccaccaagctgggcgcccgcaacatgaccttcgtcgtcagcggcgtcctcctgctcaactacctcgtctcgatctccatcggcatcatctggccccaggtcttcaagtccaacatcatgatcctcagccacgccatcctggccttctgcctcatcttccagacccgcgagctggccctcgccaactacgcctccgccccgagccgccagttcttcgagttcatctggctcctctactacgccgagtacttcgtctacgtcttcatctgattaattaaggcaggcaggagttggagtatgagggtagccgctgatggctattcttcccacgtttttgtgtgtttcctcttcatttttttttctcttgccgcaacatgacggctcctgtctctgaagggaacccctgaaattcagggttatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtccgcgatgagcagagcattttaaaggaacgccgcactcacaaacaccaacactttagtgtctagtctacagaggcgtccctccccgtcttggatgcgtgattccattaccgtagatagtaccgcaaatgcacgggggtgtagtgtatgaaccacgctgggttcctgacctgacccggcaacccaatggagcagactcagggcccgctggccccggtggcgtatcaggtgactgttgggggagctaaccttggcaaacaaccgagctcagcgttaatgcatttcaagaagtcggtttgattgatcatccgcgaggaccgattatcgtacggcatcgaaaatcgtctcgccggagcgcacggattatttgaagaggctggcttgttgattgcaattgtcggctgccggccacgtcaccggccttgcagggcttatcagtaaatgcgggggcggagcagaggcggttcttgtcaagggtaggaggggtccggcaaagcccgagacggtggctgttcggaaacccaagaatggaccctgacagaacaattttcggattgggttcgttgcaaggatcgaacactacatcttccgagagagtttggaggttgtaagaacccttcgctaccgggagaacaaatcaccttgttgaatcagctctgtcactgctagtggcgagatggcctaagcagcgagactgttccccctgccccgctgtggatccgcatgactggtccattctggtcacttcgctccacttctctgcttttgcattgaccgctcagcggctgttgcgccttcctgacgcattcatagccccactcctgggcggcagcctggcgcttccaccatgcttgcccaacacgtatataaccttctcggcctaccctctaccacggagccactttctcttctccaacatcctccacacaacacccttctccttcgccatcaaagaggcatctatcggaaaatccaacatcgccagactcaccgaaacttcatacactcataacaactgcaaccatgaaccacctgcgcgccgagggcccggcctcggtcctcgccatcggcaccgccaaccccgagaacatcctcctgcaggacgagttcccggactactacttccgcgtcaccaagtccgagcacatgacccagctgaaggagaagttccgcaagatctgcgacaagagcatgatccgcaagcgcaactgcttcctcaacgaggagcacctgaagcagaacccgcgcctcgtcgagcacgagatgcagaccctggacgcccgccaggacatgctggtcgtcgaggtccccaagctgggcaaggacgcctgcgccaaggccatcaaggagtggggccagccgaagtcgaagatcacccacctgatcttcacctcggcctccaccaccgacatgccgggcgccgactaccactgcgccaagctgctgggcctctccccctcggtcaagcgcgtcatgatgtaccagctgggctgctacggtggcggcaccgtcctccgcatcgccaaggacatcgccgagaacaacaagggcgcccgcgtcctggccgtctgctgcgacatcatggcctgcctgttccgcggcccctccgagtcggacctggagctcctggtcggccaggccatcttcggcgacggcgccgccgccgtcatcgtcggcgccgagcccgacgagtcggtcggcgagcgcccgatcttcgagctggtcagcaccggccagaccatcctgcccaactcggagggcaccatcggcggccacatccgcgaggccggcctcatcttcgacctgcacaaggacgtcccgatgctgatctcgaacaacatcgagaagtgcctcatcgaggccttcacccccatcggcatcagcgactggaactcgatcttctggatcacccaccctggcggcaaggccatcctcgacaaggtcgaggagaagctccacctgaagtccgacaagttcgtcgactcccgccacgtcctgtcggagcacggcaacatgagctcgtccaccgtcctcttcgtcatggacgagctccgcaagcgctcgctggaggaaggcaagtcgaccaccggcgacggcttcgagtggggcgtcctgttcggcttcggcccgggcctcaccgtcgagcgcgtcgtcgtccgcagcgtcccgatcaagtactaacgcgcgcgagtgtctgcatcggacgggaatgggcctgggagcgttttagcgggtttgggacggccaaccattggctgccgctggaaatttggggtttaccattaatgacacggtaacatggagataccacggatgaatagactcgtttggagtcccccgattattgttcgtttgatgctgcgtaatcgtggtgcgatgacatttgatgcctatgggatggcgggggtctcccccgctttcggaagttgcatgtgaaaaacagttcctgctccgtcctagccttggcaatgcaaacttggatgttccggcttcgtaaccgcctttcacatccttcctccgacaatgcaggttgttgccgacaagccagcacgtcaatgatcctcatgatgcagcttgctgcaagagagcgcaagcttcgagaagcagagcattcattacctcccgtgcctccgtgaacacgtctcgtctcgtcggtcaaagttttgccaccatcatcctacactcggcgcgccctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcggtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggttt

SEQ ID NO: 49 - DNA, 14419 bp, Artificial Sequence
  Synthetic Polynucleotide
gtttaaacaaccgactaacgaccgttctattattattttttcttcttccccgccaggttcaatcttagcaacgccgctaccagcctacctccgggtgccgctgcgatactaccgcgcgctgacggtcccctaccacgacgacgagaccaacagccagatccaccgcgtctaccagccggcgggcgaggagcacgcaatgcggcacttctgcgggttctgcggcaccccgctctcgtactggtccgagtcgccgcgcagcgaggccgacttcatccgcctgaccctgggcagcctgctgcatgaagacctaagagacctggaggaatgggggttggtccccgatcccgactccgcctccggcacggggacgcctttggaacaagaagatgaggcggcggaaggaaaccggggcaaaagtggggaggggaagacgcgtacggacgctgcgacgggagctgagggagaaagggaggtcggggaagcgggggggaatgtttggggcagggtcggggtgctgccgtggttcgagaccctcacggagggcagccggctagggacgaccttgcgacgggcgagaggtggggggacagacccaacggaaagggtgaggattgagtgggagattgctgagtggagtgccgaggacgaaaagggtaatgagagtccacgtaagcggaagttggatgaggttgaggacgccgttgaggccgagaggacggtgggcgtgcgtgtacaatagagtgatgtggttgcctcgcatgcaagacggcaaacgcacacccgtgccatgcatgccacgggtaaggggtgaggagattggtctgcgtggggggcatataagaccttaatttagggctctctatgatatcgaccggcaagaatcctggacatctcactcgctacaaggtgcgcttgcttcttggacgcagctagctgatgatgtttcgcatcttcaacctgcctccaaacagcgacaatgcagttgcatcttcgtgtagaagagccgcgcggttaatcttcaatccaccgagtacggtagatcaatccaggcaataactactgcgccggcgagtgtaggatgatggtggcaaaactttgaccgacgagacgagacgtgttcacggaggcacgggaggtaatgaatgctctgcttctcgaagcttgcgctctcttgcagcaagctgcatcatgaggatcattgacgtgctggcttgtcggcaacaacctgcattgtcggaggaaggatgtgaaaggcggttacgaagccggaacatccaagtttgcattgccaaggctaggacggagcaggaactgtttttcacatgcaacttccgaaagcgggggagacccccgccatcccataggcatcaaatgtcatcgcaccacgattacgcagcatcaaacgaacaataatcgggggactccaaacgagtctattcatccgtggtatctccatgttaccgtgtcattaatggtaaaccccaaatttccagcggcagccaatggttggccgtcccaaacccgctaaaacgctcccaggcccattcccgtccgatgcagacactatttaaattacttgctgcgcttgtagaccttgttgaggaaggcggtcaggacgtcggccttgaagccgcgcgactcgtcgacctgggagatcttggccttgaggtccttggcgatcgactcctcgtactcgtggtagagctgctcgatcttcaggtcgttgaagatcttcttgcacttggcctcggcgacgctgtccttcttgccgtagttctcgtcgagggtcttgcgctgctcggccgaggccagctccagggccttgttgatgacccaggagcacttgttgtcctggatgtcggtgccgatcttgccgatctgctcgggggtgccgaagcagtcgaggtagtcgtcctggatctggaagtactcgccgagggggatcaggacgtcgcgggcctgcttcaggtccttctcgtcggtgatgccggcgacgtacatggccagggcgaccggcaggtagaacgagtagtaggcggtctcgaaggtgacgatgaagctgtgcttcttgagcgagaacttgctcaggtcgaccttgtcctccggggcggtgatgaggtccatcagctggccgagctcggtctggaaggtgacctcgtggaagagctcggtgatgtcgatgtagtacttctcgttgcggaagtggctcttcaggagcttgtagatggcggcctccagcatgaaggcgtcgttgatggcgatctcgccgacctcggggaccttgtaccagcagggctggcccctgcgggtgatcgacttgtccatcatgtcgtcggcgacgaggaagtaggcctgcaggagctcgatgcaccagcccaggatggcgaccttctcgtactcttcctggccgagctgctcgacggtcttgttggacaggatggcgtaggtgtcgacgacggagaggccgcggttcagcttgccgccaggggtgttgtagttgagggagtgggcgtaccagtcgcaggcttccttcggcatgccgtaggccaggagcgaggcgttgagctcctcgaccagcttggggaagacgttcaggaagcgctcgcggcggatctccttctcggaggccatggttgcagttgttatgagtgtatgaagtttcggtgagtctggcgatgttggattttccgatagatgcctctttgatggcgaaggagaagggtgttgtgtggaggatgttggagaagagaaagtggctccgtggtagagggtaggccgagaaggttatatacgtgttgggcaagcatggtggaagcgccaggctgccgcccaggagtggggctatgaatgcgtcaggaaggcgcaacagccgctgagcggtcaatgcaaaagcagagaagtggagcgaagtgaccagaatggaccagtcatgcggatccacagcggggcagggggaacagtctcgctgcttaggccatctcgccactagcagtgacagagctgattcaacaaggtgatttgttctcccggtagcgaagggttcttacaacctccaaactctctcggaagatgtagtgttcgatccttgcaacgaacccaatccgaaaattgttctgtcagggtccattcttgggtttccgaacagccaccgtctcgggctttgccggacccctcctacccttgacaagaaccgcctctgctccgcccccgcatttactgataagccctgcaaggccggtgacgtggccggcagccgacaattgcaatcaacaagccagcctcttcaaataatccgtgcgctccggcgagacgattttcgatgccgtacgataatcggtcctcgcggatgatcaatcaaaccgacttcttgaaatgcattaacgctgagctcggttgtttgccaaggttagctcccccaacagtcacctgatacgccaccggggccagcgggccctgagtctgctccattgggttgccgggtcaggtcaggaacccagcgtggttcatacactacacccccgtgcatttgcggtactatctacggtaatggaatcacgcatccaagacggggagggacgcctctgtagactagacactaaagtgttggtgtttgtgagtgcggcgttcctttaaaatgctctgctcatcgcgctagcatttccacaactcatgccgagagaagtgtcagactgggcaactaaagtagtagtagtaatagctcgattaccatgatgaaatgctgggcgtcgaagcagctgcaggtccggcatgcagcaatccccactccgctccatcggctgctgttctggggtcaatccgtaccctcccaagttcacctcgccgctgacctcgggatcagctgcgttgtgcattcatgaataatgcgcaacatgagcaacccaacttcatcaagggagtttcgacgtcaccatatcaccacacatcttggaacagaattggggacaaggcagctggattgacggggaaatacataaaccggacgacctatgacgaccatcctatcccgtcaccgactccgatcccctgcggatgatggctcaaagaaccaagtttgatgagccggctgtgtgtcccagcacatacaacaaccgaagtatcagccccgttccgaacgcaggatcccagtctaccgaatcgattttggacagcccgagagaagccaaacaccgaaccgaaggaggaattgtcggaagacgtgcattatgagtcgttcattaattaattacgacttgatgcaggtgacgctgccgtccttcaggcggttgatgtcggtggcgtcgaggttgttgggcttggtgggctcggccggcttgcggttgtgggtcatgtggctctggaccaggtggccggcggcgagggcggcgcacagggagagctcgccggccaggacggcgcaggcgacgatgcgggcgagctggcgggcgttggtgccgggggcggtggcgtgggggccgcggacgcccaggaggtccagcatggcgccctggggctcgaggacggtgccgccgccgatggtgccgacctcgatcgacggcatgctgacggagatgcgcaggtcgccgtcgacttccttcatgagggtgatgcagttcgagctctcgacgttctgggccgggtcctggcccagggcgaggaagacggcggtgacgaggttggcggcgtgggcgttgaagccgccgaccgagccggccatggccgagccgaccaggttcttggcgatgttcagctcgacgagggcggagacgtccgacttgaggaccttgcggacgacgtcgcccgggatggtggcctcggcgacgaccgacttgccgcggccctcgatccagttgatggcggcgggcttcttgtcggtgcagtagttgcccgagacgctgacgacctccatgtcctcccagccgtactcctcgaccatctgcttgagggagtactcgacgcccttcgagatcatgttcatgcccatggcgtcgccggtggtggtgcggaagcgcatgaacaggaggtcgccggccaggcaggtctggatgtgctggaggcgggcgaagcgcgaggtggagttgaaggccttcttgatggcgttctggccttcctcggagtccagccagatcttgcaggcgccggagcgcttgagggtggggaagcggacgacggggccgcgggtcatgccgtccttggtcaggacggtggtggcgccgccgccagcgttgatggccttgcagccgcgcatggccgaggcgaccaggcagccctcggtggtggccatggggatgtggtacgaggtgccgtcgatgaccagggggccgatgacgccgacggggagcggcatgtagccgatgacgttctcgcagcaggcgccgaagacgcggtcgtagtcgtagttcttgtagggcaggcggtcggaggccaggacgggggcctcggccaggatgctgagggccttcctgcggacggcgacggcgcgggtggtgtcgcccagcttcttctcgagggcgtacagcgggagcttgccgtggatgaccagggcggcgacttccttgttcttgagctgcttggtgttgccggaggacaggagggcctccagctcctccagggggcggatcttcttgtcgagcgactcgatgtcgcggctgtcgtcttcctccgaggaggagctggggcccgaggaggacgactgggcggaggacaggctcttgaccttgctgccggagatgacggtcttgttggtgaggacgggggtgctggccttctggacgggggcggtgaaggacttcttggtgacctcggtcttgacgagctggtccatgttgcttgcgtggtgtgctggaagctgagtgtattaggtggattgacaagtccctgcgggcaacgggaccgagtgagcaagccaggatcaggcgagcaagaggcaggtggtctgattctatcaacctacgtttagagacttgagatggaccagggaatgggcgttttgttttcgaattgatggtttacgatggatttcgttggacggaagaccgatgaggggaaaggagaggagaagcccaaagagggggtcggaggtggcctttattaagaggcggccggccgggcaatgggcagatcagccatctttgctgcatcgttcctgcgactgtttcgtcagaccgggcggggtaatgtcaggagagctccctggtaggttgcgggcagcggcagtgatcacgttgactggctcacgggatcgcgtgacgagtacatcatgatggcacgacctcgcaggcgagccctgcgtggcttaaacaggccaaggtacctggcccgagcgtcctgcagccagcgctaacagcccagcccaagccagaatctggggtaatctggggtaccggggtgcccgacccactgcgggcaaccagcgcttgtgcaccgcgtaaggcctcaacaagacatcagttagtatcgatgccgagattcagttggcaattacatacgtctaacttttccaatgcttattttgagtttcttgtagttatgcagctggtggaagttaggacaggaaccttgagtgacaagcaatccggccgggccgggaaggtgcccgctgcacgatcagtggggcaaaggtggggtatcccgagcaggagcgaactccaacagagtattcgatcaaaaaaggcaagtcctcccccaccatcctttgtagcttgcaatgcatctccttttttgcaatggattttgcttcgcgagtgctaatgccttgtgaaggactatgtggttggttcaaacctgttgttttgatccatctagtccacgttgcaggcatacaaatacccgacggcgcgccctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcggtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 50 - DNA, 14419 bp, Artificial Sequence
  Synthetic Polynucleotide
gtttaaacaaccgactaacgaccgttctattattattttttcttcttccccgccaggttcaatcttagcaacgccgctaccagcctacctccgggtgccgctgcgatactaccgcgcgctgacggtcccctaccacgacgacgagaccaacagccagatccaccgcgtctaccagccggcgggcgaggagcacgcaatgcggcacttctgcgggttctgcggcaccccgctctcgtactggtccgagtcgccgcgcagcgaggccgacttcatccgcctgaccctgggcagcctgctgcatgaagacctaagagacctggaggaatgggggttggtccccgatcccgactccgcctccggcacggggacgcctttggaacaagaagatgaggcggcggaaggaaaccggggcaaaagtggggaggggaagacgcgtacggacgctgcgacgggagctgagggagaaagggaggtcggggaagcgggggggaatgtttggggcagggtcggggtgctgccgtggttcgagaccctcacggagggcagccggctagggacgaccttgcgacgggcgagaggtggggggacagacccaacggaaagggtgaggattgagtgggagattgctgagtggagtgccgaggacgaaaagggtaatgagagtccacgtaagcggaagttggatgaggttgaggacgccgttgaggccgagaggacggtgggcgtgcgtgtacaatagagtgatgtggttgcctcgcatgcaagacggcaaacgcacacccgtgccatgcatgccacgggtaaggggtgaggagattggtctgcgtggggggcatataagaccttaatttagggctctctatgatatcgaccggcaagaatcctggacatctcactcgctacaaggtgcgcttgcttcttggacgcagctagctgatgatgtttcgcatcttcaacctgcctccaaacagcgacaatgcagttgcatcttcgtgtagaagagccgcgcggttaatcttcaatccaccgagtacggtagatcaatccaggcaataactactgcgccggcgagtgtaggatgatggtggcaaaactttgaccgacgagacgagacgtgttcacggaggcacgggaggtaatgaatgctctgcttctcgaagcttgcgctctcttgcagcaagctgcatcatgaggatcattgacgtgctggcttgtcggcaacaacctgcattgtcggaggaaggatgtgaaaggcggttacgaagccggaacatccaagtttgcattgccaaggctaggacggagcaggaactgtttttcacatgcaacttccgaaagcgggggagacccccgccatcccataggcatcaaatgtcatcgcaccacgattacgcagcatcaaacgaacaataatcgggggactccaaacgagtctattcatccgtggtatctccatgttaccgtgtcattaatggtaaaccccaaatttccagcggcagccaatggttggccgtcccaaacccgctaaaacgctcccaggcccattcccgtccgatgcagacactatttaaattacttgctgcgcttgtagaccttgttgaggaaggcggtcaggacgtcggccttgaagccgcgcgactcgtcgacctgggagatcttggccttgaggtccttggcgatcgactcctcgtactcgtggtagagctgctcgatcttcaggtcgttgaagatcttcttgcacttggcctcggcgacgctgtccttcttgccgtagttctcgtcgagggtcttgcgctgctcggccgaggccagctccagggccttgttgatgacccaggagcacttgttgtcctggatgtcggtgccgatcttgccgatctgctcgggggtgccgaagcagtcgaggtagtcgtcctggatctggaagtactcgccgagggggatcaggacgtcgcgggcctgcttcaggtccttctcgtcggtgatgccggcgacgtacatggccagggcgaccggcaggtagaacgagtagtaggcggtcttgaaggtgacgatgaagctgtgcttcttgagcgagaacttgctcaggtcgaccttgtcctccggggcggtgatgaggtccatcagctggccgagctcggtctggaaggtgacctcgtggaagagctcggtgatgtcgatgtagtacttctcgttgcggaagtggctcttcaggagcttgtagatggcggcctccagcatgaaggcgtcccagatggcgatctcgccgacctcggggaccttgtaccagcagggctggcccctgcgggtgatcgacttgtccatcatgtcgtcggcgacgagccagtaggcctgcaggagctcgatgcaccagcccaggatggcgaccttctcgtactcttcctggccgagctgctcgacggtcttgttggacaggatggcgtaggtgtcgacgacggagaggccgcggttcagcttgccgccaggggtgttgtagttgagggagtgggcgtaccagtcgcaggcttccttcggcatgccgtaggccaggagcgaggcgttgagctcctcgaccagcttggggaagacgttcaggaagcgctcgcggcggatctccttctcggaggccatggttgcagttgttatgagtgtatgaagtttcggtgagtctggcgatgttggattttccgatagatgcctctttgatggcgaaggagaagggtgttgtgtggaggatgttggagaagagaaagtggctccgtggtagagggtaggccgagaaggttatatacgtgttgggcaagcatggtggaagcgccaggctgccgcccaggagtggggctatgaatgcgtcaggaaggcgcaacagccgctgagcggtcaatgcaaaagcagagaagtggagcgaagtgaccagaatggaccagtcatgcggatccacagcggggcagggggaacagtctcgctgcttaggccatctcgccactagcagtgacagagctgattcaacaaggtgatttgttctcccggtagcgaagggttcttacaacctccaaactctctcggaagatgtagtgttcgatccttgcaacgaacccaatccgaaaattgttctgtcagggtccattcttgggtttccgaacagccaccgtctcgggctttgccggacccctcctacccttgacaagaaccgcctctgctccgcccccgcatttactgataagccctgcaaggccggtgacgtggccggcagccgacaattgcaatcaacaagccagcctcttcaaataatccgtgcgctccggcgagacgattttcgatgccgtacgataatcggtcctcgcggatgatcaatcaaaccgacttcttgaaatgcattaacgctgagctcggttgtttgccaaggttagctcccccaacagtcacctgatacgccaccggggccagcgggccctgagtctgctccattgggttgccgggtcaggtcaggaacccagcgtggttcatacactacacccccgtgcatttgcggtactatctacggtaatggaatcacgcatccaagacggggagggacgcctctgtagactagacactaaagtgttggtgtttgtgagtgcggcgttcctttaaaatgctctgctcatcgcgctagcatttccacaactcatgccgagagaagtgtcagactgggcaactaaagtagtagtagtaatagctcgattaccatgatgaaatgctgggcgtcgaagcagctgcaggtccggcatgcagcaatccccactccgctccatcggctgctgttctggggtcaatccgtaccctcccaagttcacctcgccgctgacctcgggatcagctgcgttgtgcattcatgaataatgcgcaacatgagcaacccaacttcatcaagggagtttcgacgtcaccatatcaccacacatcttggaacagaattggggacaaggcagctggattgacggggaaatacataaaccggacgacctatgacgaccatcctatcccgtcaccgactccgatcccctgcggatgatggctcaaagaaccaagtttgatgagccggctgtgtgtcccagcacatacaacaaccgaagtatcagccccgttccgaacgcaggatcccagtctaccgaatcgattttggacagcccgagagaagccaaacaccgaaccgaaggaggaattgtcggaagacgtgcattatgagtcgttcattaattaattacgacttgatgcaggtgacgctgccgtccttcaggcggttgatgtcggtggcgtcgaggttgttgggcttggtgggctcggccggcttgcggttgtgggtcatgtggctctggaccaggtggccggcggcgagggcggcgcacagggagagctcgccggccaggacggcgcaggcgacgatgcgggcgagctggcgggcgttggtgccgggggcggtggcgtgggggccgcggacgcccaggaggtccagcatggcgccctggggctcgaggacggtgccgccgccgatggtgccgacctcgatcgacggcatgctgacggagatgcgcaggtcgccgtcgacttccttcatgagggtgatgcagttcgagctctcgacgttctgggccgggtcctggcccagggcgaggaagacggcggtgacgaggttggcggcgtgggcgttgaagccgccgaccgagccggccatggccgagccgaccaggttcttggcgatgttcagctcgacgagggcggagacgtccgacttgaggaccttgcggacgacgtcgcccgggatggtggcctcggcgacgaccgacttgccgcggccctcgatccagttgatggcggcgggcttcttgtcggtgcagtagttgcccgagacgctgacgacctccatgtcctcccagccgtactcctcgaccatctgcttgagggagtactcgacgcccttcgagatcatgttcatgcccatggcgtcgccggtggtggtgcggaagcgcatgaacaggaggtcgccggccaggcaggtctggatgtgctggaggcgggcgaagcgcgaggtggagttgaaggccttcttgatggcgttctggccttcctcggagtccagccagatcttgcaggcgccggagcgcttgagggtggggaagcggacgacggggccgcgggtcatgccgtccttggtcaggacggtggtggcgccgccgccagcgttgatggccttgcagccgcgcatggccgaggcgaccaggcagccctcggtggtggccatggggatgtggtacgaggtgccgtcgatgaccagggggccgatgacgccgacggggagcggcatgtagccgatgacgttctcgcagcaggcgccgaagacgcggtcgtagtcgtagttcttgtagggcaggcggtcggaggccaggacgggggcctcggccaggatgctgagggccttcctgcggacggcgacggcgcgggtggtgtcgcccagcttcttctcgagggcgtacagcgggagcttgccgtggatgaccagggcggcgacttccttgttcttgagctgcttggtgttgccggaggacaggagggcctccagctcctccagggggcggatcttcttgtcgagcgactcgatgtcgcggctgtcgtcttcctccgaggaggagctggggcccgaggaggacgactgggcggaggacaggctcttgaccttgctgccggagatgacggtcttgttggtgaggacgggggtgctggccttctggacgggggcggtgaaggacttcttggtgacctcggtcttgacgagctggtccatgttgcttgcgtggtgtgctggaagctgagtgtattaggtggattgacaagtccctgcgggcaacgggaccgagtgagcaagccaggatcaggcgagcaagaggcaggtggtctgattctatcaacctacgtttagagacttgagatggaccagggaatgggcgttttgttttcgaattgatggtttacgatggatttcgttggacggaagaccgatgaggggaaaggagaggagaagcccaaagagggggtcggaggtggcctttattaagaggcggccggccgggcaatgggcagatcagccatctttgctgcatcgttcctgcgactgtttcgtcagaccgggcggggtaatgtcaggagagctccctggtaggttgcgggcagcggcagtgatcacgttgactggctcacgggatcgcgtgacgagtacatcatgatggcacgacctcgcaggcgagccctgcgtggcttaaacaggccaaggtacctggcccgagcgtcctgcagccagcgctaacagcccagcccaagccagaatctggggtaatctggggtaccggggtgcccgacccactgcgggcaaccagcgcttgtgcaccgcgtaaggcctcaacaagacatcagttagtatcgatgccgagattcagttggcaattacatacgtctaacttttccaatgcttattttgagtttcttgtagttatgcagctggtggaagttaggacaggaaccttgagtgacaagcaatccggccgggccgggaaggtgcccgctgcacgatcagtggggcaaaggtggggtatcccgagcaggagcgaactccaacagagtattcgatcaaaaaaggcaagtcctcccccaccatcctttgtagcttgcaatgcatctccttttttgcaatggattttgcttcgcgagtgctaatgccttgtgaaggactatgtggttggttcaaacctgttgttttgatccatctagtccacgttgcaggcatacaaatacccgacggcgcgccctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcggtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 51 - DNA, 12575 bp, Artificial Sequence
  Synthetic Polynucleotide
gtttaaaccccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtatttaaatctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctggcgcgcctggccaagagaaccgaccagttgccccaggacgatctagacaaaaaaaaagagagatgagtgggccacttttgccacaacatcgacggccctgcgaccgcccccaggcaaacaaacaaaccgccgaacaataatacttttgtcattttaggaggagcgttgtatggataaaaacaacatctcgttgctgcagaatgtggacttcaaacttgcagaaaatgggaggcggatttgcatgatcggagggtagttgactcacgccgcaggctgcaaatccgtcctccattattccatgaacaacttcgtaaggttgggctgagcgccaatgcctaacggaccgggggccacagcgcaacgtcccacttaaaggccagcgtgacatgccagttccataccaagtagtggcaccagaggcggccaatgctcagtaagggcagggagggaggctcaaacgattggcaaaaagaggggcttgccagttcagttccctgtgcgagcgcgagaggggcagtttcaaatctggaggggtgtgttgcgctggtctgaagagaaagagaagactgtacttaataattgttcaaagagtccatcatcgcgttgcggactcctctagctgtatttagagccctatcattacttgtcgggtgcgaatcaaaataccgggatgcagccctctggcgatttgcatgcggttgtggaggaagtgaagcctgaatcgcggggctgggcggcaaagcacgacgtgaaattcctggcgaaattcgagggcttgccccaccgtggttgaagtttttgtgctgcgtaaccccaccaacccgccttgcccctcccgcctgcccataaaaacttcgacccctcctcaaatcttcttcgattcttcctcttcacttccttcgtcggcatacctgattcaagcaatcacctgccactttcaagtgcgtataccatcatcgatacactggttcttgacaagtacatcgtctctaactttcctttttgcagttttcattaagcgcaagtcgccagtttcgttcttcagaacacaaataccgtcaaaatgggcaagaactacaagagcctggactcggtcgtcgcctccgacttcatcgccctgggcatcacctcggaggtcgccgagacgctccacggccgcctggccgagatcgtctgcaactacggcgccgccaccccgcagacctggatcaacatcgccaaccacatcctctcgcccgacctgccgttctccctccaccagatgctgttctacggctgctacaaggacttcggccccgcccctcccgcctggatcccggaccccgagaaggtcaagtccaccaacctgggcgccctcctggagaagcgcggcaaggagttcctcggcgtcaagtacaaggaccccatcagctcgttcagccacttccaggagttctcggtccgcaacccggaggtctactggcgcaccgtcctgatggacgagatgaagatcagcttctcgaaggaccccgagtgcatcctccgcagggacgacatcaacaaccctggcggctcggagtggctccctggcggctacctgaactcggccaagaactgcctgaacgtcaactccaacaagaagctcaacgacaccatgatcgtctggcgcgacgagggcaacgacgacctccccctgaacaagctcaccctcgaccagctgcgcaagcgcgtctggctggtcggctacgccctggaggagatgggcctcgagaagggctgcgccatcgccatcgacatgccgatgcacgtcgacgccgtcgtcatctacctcgccatcgtcctggccggctacgtcgtcgtcagcatcgccgactcgttctcggccccggagatctccacccgcctccgcctgagcaaggccaaggccatcttcacccaggaccacatcatccgcggcaagaagcgcatcccgctgtactcgcgcgtcgtcgaggccaagtcccccatggccatcgtcatcccctgctccggctcgaacatcggcgccgagctgcgcgacggcgacatcagctgggactacttcctcgagcgcgccaaggagttcaagaactgcgagttcaccgcccgcgagcagccggtcgacgcctacaccaacatcctcttctcctcgggcaccaccggcgagcccaaggccatcccctggacccaggccaccccgctgaaggccgccgccgacggctggtcgcacctcgacatccgcaagggcgacgtcatcgtctggcccaccaacctgggctggatgatgggcccctggctggtctacgcctccctcctgaacggcgcctccatcgccctgtacaacggcagccccctcgtctcgggcttcgccaagttcgtccaggacgccaaggtcaccatgctgggcgtcgtcccctccatcgtccgctcctggaagagcaccaactgcgtctccggctacgactggagcaccatccgctgcttctcctcgtcgggcgaggccagcaacgtcgacgagtacctctggctgatgggccgcgccaactacaagcccgtcatcgagatgtgcggtggcaccgagatcggcggcgccttctccgccggctccttcctgcaggcccagtccctctcgtccttcagctcgcagtgcatgggctgcaccctctacatcctggacaagaacggctaccccatgccgaagaacaagccgggcatcggcgagctggccctgggcccggtcatgttcggcgcctcgaagaccctcctgaacggcaaccaccacgacgtctacttcaagggcatgcccaccctcaacggcgaggtcctccgcaggcacggcgacatcttcgagctcacctccaacggctactaccacgcccacggccgcgccgacgacaccatgaacatcggcggcatcaagatctccagcatcgagatcgagcgcgtctgcaacgaggtcgacgaccgcgtcttcgagacgaccgccatcggcgtcccgcccctcggcggcggccccgagcagctggtcatcttcttcgtcctcaaggacagcaacgacaccaccatcgacctcaaccagctccgcctgtcgttcaacctcggcctgcagaagaagctcaacccgctgttcaaggtcacccgcgtcgtccccctctcctccctgccccgcaccgccaccaacaagatcatgcgcagggtcctccgccagcagttcagccacttcgagtaacgcgcgcgtgaacgactcataatgcacgtcttccgacaattcctccttcggttcggtgtttggcttctctcgggctgtccaaaatcgattcggtagactgggatcctgcgttcggaacggggctgatacttcggttgttgtatgtgctgggacacacagccggctcatcaaacttggttctttgagccatcatccgcaggggatcggagtcggtgacgggataggatggtcgtcataggtcgtccggtttatgtatttccccgtcaatccagctgccttgtccccaattctgttccaagatgtgtggtgatatggtgacgtcgaaactcccttgatgaagttgggttgctcatgttgcgcattattcatgaatgcacaacgcagctgatcccgaggtcagcggcgaggtgaacttgggagggtacggattgaccccagaacagcagccgatggagcggagtggggattgctgcatgccggacctgcagctgcttcgacgcccagcatttcatcatggtaatcgagctattactactactactttagttgcccagtctgacacttctctcggcatgagttgtggaaatgcgccggcgaatgccctccgtccccttccttcacaacctcgaattcctcccaatgtggatatctgtcgcctttctaagaaagggcgtggaacacgcgcgattagtatagaatatggatcgacctaagttgtctccgcacatgtctcaacagtctagcgacaagaagaacctcgcccacccgtcgattacgagcgtgtgcagcctgagtgtgtgtgagttggagttaacggcgccgaaatctgaaggagggaagagacttttcaacacgtctgttctctactgactttttttgtttttaccacatcgcactaggaaagctagcggtgttttgatggtgccaatactgcgtaactgcgtaatgttgcatattgcgtagcagcgtgaagcaagtggatgtatgtacggactaatccgtatgcactgcatctcgcagcagatggcacctccccagagacagccgggaaacaagcttttttttccttggcgtccttggcttgcatggcttcattggcgggtgttatgttttccccaggggtgcagcaatggcacgccgagcaaaaaaggaaccgcggacctggcacaagccccaaactccatcgacgcaggacggcatcgcattgcgttgcgcctcctctccaacgacgtcttaagggaaaagaaaagaaaaacaaaaggataatggcaggcctccagcaagcaagcaagagcgcttccggccgttcaagggtccaatccggttcaagccttgcgttatcgtcccagagggcggcccttgttgtaagccggccttgtgttcgcgcccttcgatgtttgacattgcttttccgtctgggtactttccagtcggttgttggagacttcctcgtcattgtatggggtcacatgttctctgcgcactacactacggagtaagtgctaataaattacatctcgaccccgtgcttggcaacaacctcgaggaacctgtcctgcttgccttatttccgtcggttgggtagacggcttgttcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 52 - DNA, 17183 bp, Artificial Sequence
  Synthetic Polynucleotide
gtttaaactcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaaggagaatttaaatttatcggacggcctgatttgcggcatgcctcatcgcggcgaaactgctgataagacctgctgccgcagtccgcccattcctgtgtctgacgcacaccgattggggcatgtccgcggaaaaagagacgatcctctccactaggtggtttccgcctttaacggatcgaggaaacattagttgttagtagtaacacgccttgaacgccttgatctccggggctcctcctcggacgaccgcggaccgccggtcagagtgggtgggtacaccacactacacgatactatcgcgcatcgtcgttgtccctgctctctccccatatggcgtcacggggtagtatatattctacacggagctggagccaaggcaggcccaccgtacggattacaccgccatggttccaagtttcttcgccattgaatctgttattcgtgtactagtagatcaagccattgccggttgccgatatcatgcaactccggtcaggccacggagaagccatgggcgcgccatgagcgcgaatgaacactacgttggagaagaactcgacacatccgacaagacctcacatacccagacctgcccaggtggtatttgatatcaggatcaatggcgtcggacattgctacagcactcttgacgcctgtgaccgaatcgctccagcgctacaaattacccagtagccgggcactaacagctccctggcctaggtagactacctacctcaaggtacgacacatggcagcactggagggggaataggcagactggacgacagtggacaagatacggtcgcacaacctttgtcgtggcatcgcgagaataatcgtcacaagcttcacgtatgcagacggagacaagatgatttggttgtcgaagtcatgaattcacttctatctagtttttttgttcccttttgttttgcattcccagagaagttctgatggaacccttattcccagcctctcaattaacgtgcctcgattcatagtcgagtgctcatgcatagcaacattgatcgtttcgtcgtagaagtgagcgcatggtggtgcccacctggagaaacctcacgagggaccccagaacatcaggtgttgatgatgggtatcgcggccggcctcagccgccgtactcgtcaatgcccagggcgtcggtgaacatctgctcaaactcgaagtcggccatgtccagggcgccgtagggggcggagtcgtggggggtaaagccggggccggggctgtcgccgtcgcccagcatgtcgaggtcgaagtcgtcgagggcgtcggcgtgggccatggcgacgtcctcgccgtccaggtggagctcgtcgccgagggagacgtccgtggggggggccgtgctgaccttgcgcttcttcttgggagggctagccgattgtcgggagagagcagcccagagcgattcctctacccccgtaagcaactcatccgttagagagagataatcgttttcgatcatctcatagacctccataaacgatccgaataggatggcgatcagggcattctcgggcaagtttcgaattacgccctgtttctgtccctctcgaaagaaggtgcagacgaactcaacaagtttttggtatgcaaggcgtgactcttcggttaggaatgtaccttgggaatgtgtcttgataaatcccaaggcgcgcggatggttctttgtgaatgtgaccattccctcgaagatatgatggaacccatcgcgataaccgtccctttcgttcgccaagccactctcgatacattgcaaaaattcattaacgtgctgctggaacagctcgttgaccagactctccttattcttaaagtatcggtaaatcgttcctgcgccgaccttagcattttcagcgatcatcggcatcgtagtggcgtcaaacccgcgttcggcgaacagaaggagcgaggcagaaaaaatagctttttgtttcgtgggtgtggactccattgttaatttttttcaaatatgaatttgattgatgggagagggaaaataaaagaagaagaaaaccgaatagaaagaaggtagagagaaaacgaagaatcgaccaagtgggggcgggggggaggagggaaagtggttgatttatagtttggaattcagggcgagaccaattactacagacttattactttgggttacgggcaggtgagaaatcgatgtgtgtctccgtcacaaacctctgcagcagctcgcggccggcgaacatcggtatgtaagttaggtaggttgacaagacgggcatggtgcaggtgtggttggtaggggagatgtctagcctgaagccatacgaagaatgtattgtccatgtgtatgtacaagcgaagcgaatgaaatgcccaaaccgggggcaaaacactgaacgataggtacaacatgaaagaaacaccatgcatcgatgtgtgtgctcgcaagcataccactggtcctcccgagccccaagaagcggtcccgcaaagcgaaggaggaggaggaggaggaagaagaagaagaagaagaagaagaagaagaagtgaaaagagaaatgagaagaagaaaaaaggcccagggccaaacaatgaacaacaagaggaaaacaaaacaaaaaatatgtataacgaatggagagaaaaaagaaaagcggctaataataatgcaaaccatccaagaagagaaaacgaaggtaaacatgtccaggcttcaaaatggtttgccgaatcagtacggcttcatgctccctctctctctctctctctctctctctccagctcccctctctcgaccaggactcgggaggtcatagggccttaattaattaggccatctgctggagcaggctgtggaggcgggggctgcccgtgatgccgtggaccagctggacgtacgacttgtcgacgctgaacggctggccgacgacgttggggatccagcggccgaccagctcgcagggcttgacgtcgccgaccttgatgcgctcggagaggtactcgcggtagggctcgatctcgccgcggagcatggtcgagtggtacgggatgtcgatgccggggagggggatggtggcgcggccgcgctccatgcggtcctcgcgcgggacctgctcgacggtggggacgtgcttccagaccatggcgcggagctcctggccctcgaccgtctcgggctgggggtggcaggacaggtcgtcgcagatcttgccgagcatccacagggcgcggaagtggccggcgcagacgtactgctgcgagttgatgttgtagttgacgacctcgacgaaccagcccgtctcctgctggatgatgtggacgaggcacttcaggctggcttcctcgaagcccttgccgatgcgggaggggtcggcggccagcatgccgtagtcggtgtggccgttggcgtcgcgggggagggcgttctgcatcttcaggccgcggtagaagatgagcgagatcaggtcctcgaaggagaggaacgaggcgcaggcgccgagggcggcgtactcgcccaggctgtggccggcgaagcgggcgcccttctggacgacgccctgggccttgagccactcgaactgggccatctccatgagggccagggccggctgggcgaactgggtcgacatgagcaggccctgcgagtagctgaaggtgtaggaggtcgagttgcgggtgaggcccttcaggatgggcgggtggcggccgtcgatgggaggctggcccatcatgcggaggtagttggcgcggatgcggcggccgcgctggctgccgaagtggacggtgagggcgggcgggttgttctggacgatgtgcaggatggagaagccgtacttctcccagaggtgcttgtcggcgcgggcccacagggccttggcctccgggcagttgacgtagaggtccatgcccatgccctggcgctggctgccctggccgcagaagacgtaggcggtcgtctcctgctcgacgtgggcgtcggcctcggcgacgcgctcctcggtgcgctcgttgaaggcctggaccttgaggaccatctcgccgtcctccatggccttgtgctggagctcgacgcgcagcgggtcgttggggtggaccggggcctgcagggtgatgtgccacgagcggaagcgcgagcggtcggcgtcgccgatggcccactcggcgatcctgcgcatcatggccgacgtctccatgccgtggacgatggggccgctgaggccggcgtagcgggcgaaggcggggcagacgtggatcgggttgtggtccagcgagacgcgggcgtagctctgggagcggcgggggccgcggacggcgacggtgctggtgccggtccagccggggtgctggagctcgagcagctgggccctgggggcgccgtagcggtgcaggaagtccatgacgacgttgccggtgcacgactcgctctcgaagtagacgcggccgaaggcggtggtggagccgtccggcgagtacgagaagacgctgccggtgacctggagcagggccagctggccgtcggggcggaagagcttctcggacttcaggcggaagagcagctggcggcccagcaggtccagggcccggtcctcgcgcatgagccacttgcgcgagtgcaggatggccctgcggacctcggagtcgacgtggacgaccatctcgggctcctcggtgagctcgaacggcgtctcgcaggccaggaccgggccgcccaggaagaagtccgacttgacggtgacgacgtgctggccctggcgctggatgtcggccgagacggtgagcatggtgccgcgggggcggacgctgacggccaggatgcgcgagctggtcttgacgatgtcgccgacgcggaggggcttgacggagggggcgtagtggaagctgatggcggagtggagcaggtcgagcaggtcgcacttcagggagctgaccatgaggggcttggtcagggcgctccaggcgatgacgacgcagtagtcgatggggacgcagccctgggggttccaggactgcagctgcagcgggctggtctggcgcaggacgcgctcgaagtcgcggatcttgtcggtggtgatcatcagctcctcgccggtgaactgggagttgaggcccaggaccgaggccttgttggggaagccgaggttccacagggacatgtagagggccttgatgcgctcggcgcggccggaggcgtcttcctcgagggtccactcgttcggcttctggtcgaccttgaagcagaagacgaccgagggctccggggagagggggatgttgggggcgagggtggcgaagacgcgctggccgtcgttcgagacgatctcgaggacgaccttgctggtggagccaccgtcgccggtgggggagatcaggcggatcttgcggatctcggagtcggcggtgagcaggacctcgatggtgtcgccgcgctggagctgcagggcggcgcggatcgggttgtggaggcgcgagccgtcgcggaagacggacttcgacatcaggcaggtgcgggcccaggacttggagaggccgacgatgtgctcgaacaggacgtccatctccgggacggcgccgaccttctcgaacttgtagaggccctggacgcggttggtggtgaccttgaggccggggaaggccgacagcggcttctgggtgatcgagtggacgtcgccgatggacgtctcgcggctgtcggcctgcagggcctcgacgtagtggttgcagatgttgtgcaggatgtccttgaccgactcgtcgtcgctgatggagtactggacggccatcgggccctggatgatgaagatgcgctggacgtcctggccgatgacggcctcgacgtcctccgactgccacagggagtccttcttgaaccacgtctcgaagcgctcgtccaggcgcgggatgaaggggaccggcttgatgtccctgcggctgaagaggtgcatgagcagggagacgtcctccgggtagagggtgcggtaggccttgtccttcaggctctcctcgacgcggacgatgatgtccagggggtactcgcccgggttgtcgatggcgcactggaagcgctcgcggagcaggtggacgaagtcgagcaggaggatgcggtacgaggggtcgacccagcgcttctggtggctgacgtaggtgaggtcgcacagcctgcggaggacctccaggtaggtcatgtcctcgagctcgacgttctggccgtggccgtcgacggcgaaccagggcctggcgaagtcggcgttcaggcgggagacgatctcctggcggtggttgcggaggtactccaggcgcttcgaggtgtccttgatgctgaagacgcggttgtcgagctccttccacagcatgacgccgcgggtggccaggacgtggatgggctggccgaactccgagttgacggtgacgacgccgccggtgggctcgtcgaagctcttgtgccagtcggcgtcgccgacgccctgggcgtcgatgatgaggcgcttggcctgggccgaggtgtgggcctcgcgggcgaccatcatgcgcgagcccaggaggacgccgtcgaagggcatgcaggggtagccgaaggcctgggcccactggccggtcaggtaggggaaggtgtccgggccgccaccgaagcccgagccgacgaccaggaggatgttggggcaggagcggatctgggcgtaggtggccaggatggggccgtggaagtcctcccagctgtggtggccgccgcccctgccggcggtccactggaggccgatcaggaagttggggtgggtgcgggcgatctggatgacctggtggatggcctcgaaggagcccggcttgaacgagatgtgcttgaggccgatgctctggacgcactcctggacgacctcgggggaggggatgccggcgccgatggtgatgccctcgaccgggacgccctggcggaccaggtccttgatgacgctgatctgccaggagaaggtggtgggcttggcgtacaggaggttgcaggtgatgccgtggtcggcggggatggcggtggccaggcggcggatctcggcctcgaactggcgctcggcgtggtagccgccgccagcgagctcgacgtggtagccggcctgggcgacggcggcgacgaagtcccagcggacggtggtgggggtcatgccggcgaccatgacgggaggcttgccgagcagggtggtcatgcggttgtggagcttggcggcgccgtggacgttggcgtggaggcgcaggccgaactcggcctcccagttgacggcggccaggtggccgccgacgggcttggggcccgactgggtggtcagctggatgacggagacgccggtgccctgggtgagctcctggatcaggctgcaggtctggcccgggccgaagtccaggacgtgggtggcgttcaggccgcggcagaccaggggccagtccagctggtcgacggtgatggcgcggatgagggtcgggatcagctggtggggctggagctcctgcaggttggagccggtgccggtgtggtagagggggatcttgatgctgtggagggccagggaggccgaggagagggcctcgatgacgcggtcctggacgccgtccaggtagggggtgtggaagggggccgagatggggaggaacaggatgtcgacgatcggcttgcggttgcggaagaggatgcgggactggtccaggtcgttgtcggcgcggatgcggcggacgtggaggcagacggcccagagcgactggggcgggccggccaggacgaacttctcgtgggagttgacgagggccaggtggacccagcggttgcactcgcccaggcccttgttgacgtgctcgatgacgcgctcgacctgcgagcgcgagaggccgctgacgctcagcatcgagctcaggaggccctcgccgtgctcgatgcagtcctggatcatggcgtcggaggcggccgaggagggggtgaacaggtaggcctccaggccgatccagaagctgatctggaggacggtgcggcaggcgtcgtagaaggtgggccaggactcggcctgggtgatggcgacggcggccaggatgccctgggagtggccggtgctggagtggaggagcgagcggaactggcccgggtccagctcgagctcgcggcaggtggcgcagtagagggcgagggacaggagggtgttgagggggaagctgcggggcgggagggccaggatctccttgctgggggcgacctcgggggtggtgagccactggcgcaggtcgaagccccagggctcgtggacgtcgatggcggcgggggagctggccagctgggagagggtgttgctggcgacgtccagcagctcgtccaggtccgggccgtagcgcttgtacagctccaggaggcccttgaggacgtccaggttgttgctgccctggccaccgaaggcggcgcagatgcgggcggcgcccctctgggcggcctggatggggatggactcgtgctcgcggctgacggagcccattttaattaagttgcttgcgtggtgtgctggaagctgagtgtattaggtggattgacaagtccctgcgggcaacgggaccgagtgagcaagccaggatcaggcgagcaagaggcaggtggtctgattctatcaacctacgtttagagacttgagatggaccagggaatgggcgttttgttttcgaattgatggtttacgatggatttcgttggacggaagaccgatgaggggaaaggagaggagaagcccaaagagggggtcggaggtggcctttattaagaggcggccggccgggcaatgggcagatcatatggccacagtttccggggagaactagccggaatgaaccttcattccgattgacaagcttcagcggaatgaaagttcattccgtgcttatctagagtccggaatgaaccttcattccgtcacatcctaggtctcggaatgaatgttcattccgactagccggaatgaaccttcattccgattgacaagcttcagcggaatgaaagttcattccgtgcttatctagagtccggaatgaaccttcattccgtcacatcctaggtctcggaatgaatgttcattccgactagccgagcaaatgcctgcaaatcgctccccatttcacccaattgtagatatgctaactccagcaatgagttgatgaatctcgccggcgacgaacctctctgaaggaggttctgagacacgcgcgattcttctgtatatagttttatttttcactctggagtgcttcgctccaccagtacataaaccttttttttcacgtaacaaaatggcttcttttcagaccatgtgaaccatcttgatgccttgacctcttcagttctcactttaacgtagttcgcgtttgtctgtatgtcccagttgcatgtagttgagataaatacccctggaagtgggtctgggcctttgtgggacggagccctctttctgtggtctggagagcccgctctctaccgcctaccttcttaccacagtacactactcacacattgctgaactgacccatcataccgtactttatcctgttaattcgtggtgctgtcgactattctatttgctcaaatggagagcacattcatcggcgcagggatacacggtttatggaccccaagagtgtaaggactattattagtaatattatatgcctctaggcgccttaacttcaacaggcgagcactactaatcaacttttggtagacccaattacaaacgaccatacgtgccggaaattttgggattccgtccgctctccccaaccaagctagaagaggcaacgaacagccaatcccggtgctaattaaattatatggttcattttttttaaaaaaattttttcttcccattttcctctcgcttttctttttcgcatcgtagttgatcaaagtccaagtcaagcgagctatttgtgcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 53 - DNA, 13661 bp, Artificial Sequence
  Synthetic Polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcactagtgtttaaacgtgtgtacaatttcatacagtaatagggcccaacaggagcggtgagcgcgaaaactcttcgcagccccttaggcggcgtaccagttccgcaagcggactgaagaagagttatgctacccggctatcgccgcgtgatgtttctgattgtttctgttcgagttcggcgggccgtccccagaatagcacctccgccggaacccagactaggtacgcatgcagtgatacatcggagaatccaacgttttctagtttcccggatgctatattagagaacgctactccgtgtctgataagttctatgtaatacggagagttccgatgctgagccgttccaattccgtacttgtcgcgcatcgtgtaaagtgtttccccatgcagctctggctcgcgtcgtttgctattgtacgcgtggggaaggggagcgacccggaaagccgatgatgtacggatcagtagtgtatcgccccgcataacagtgctctcacggccattcatgccatgtgaacaaccaggccgtccggctgttcgttccggcgcgcccgcaagaccccgttgaagggacttctcggaatggggtgattatcgctgagccgggcccacttgatccactgccgacgctgctgcattcactttggtgcatacgagacagctccaagccgcttggaaaatggggtgaagggtaactatcttaatggagaacggtaatcgcatacttgcttgccctcgtcgaggcccggggctgggagaggaggggatgaaacaaaagtcaatgccgctgccgagagaccgacagcgcgatagactacctgatgaagcatggtggttgttgacacaaggatagaacggagggaggtatcacagtctaacgcagatagtatagtgtactccgtaattcatgtaaggattggtaactgcatcagaccacgacgggaccccatcatcgtggcggacctcacgggcagaagctggaggcgcccgggagagagtggatcgagttgcatgcgcaagcgcccttcatctcttcagccgagaatctgttgcctaattacatagtagatgctgggaacagtctgagaagcctgcgcaggttcttcattaattaccaagaacgcgggagaggtgggaagacgccgaaaactcgacgcggaccattcgctgttttccgatcttacaaaaaaaaggtatccgatttggggaacgtcgatgaaagtattgcaaaagtgacgagagttgcgcaactaactcgctgccgaagaagctgcggaagaaagagaacaccgaaagtggaataacgttacggatgtcctgacctcaaagttgaaaccagcccttcctgctctatttgggaaagcggcttgcccttgaatgcgctgcactgtggcacgactaccagtgatcgggaggagcaaactaccctggtccgttccttggtggggcggcactaggcccaacttaggcgccggcgagattcatcaactcattgctggagttagcatatctacaattgggtgaaatggggagcgatttgcaggcatttgctcggctagtcggaatgaacattcattccgagacctaggatgtgacggaatgaaggttcattccggactctagataagcacggaatgaactttcattccgctgaagcttgtcaatcggaatgaaggttcattccggctagtcggaatgaacattcattccgagacctaggatgtgacggaatgaaggttcattccggactctagataagcacggaatgaactttcattccgctgaagcttgtcaatcggaatgaaggttcattccggctagttctccccggaaactgtggccatatgatctgcccattgcccggccggccgcctcttaataaaggccacctccgaccccctctttgggcttctcctctcctttcccctcatcggtcttccgtccaacgaaatccatcgtaaaccatcaattcgaaaacaaaacgcccattccctggtccatctcaagtctctaaacgtaggttgatagaatcagaccacctgcctcttgctcgcctgatcctggcttgctcactcggtcccgttgcccgcagggacttgtcaatccacctaatacactcagcttccagcacaccacgcaagcaacttaattaaaatggtcatccagggcaagcggctggccgccagctccatccagctcctggcctccagcctggacgccaagaagctctgctacgagtacgacgagcgccaggccccgggcgtcacccagatcaccgaggaagccccgaccgagcagcccccgctctccaccccgcccagcctgccgcagacccccaacatcagccccatcagcgcctcgaagatcgtcatcgacgacgtcgccctctcccgcgtccagatcgtccaggccctggtcgcccgcaagctcaagaccgccatcgcccagctcccgacctccaagagcatcaaggagctgtcgggcggccgctcctccctgcagaacgagctcgtcggcgacatccacaacgagttctcgtccatccccgacgccccggagcagatcctcctccgcgacttcggcgacgccaaccccaccgtccagctgggcaagacctcctcggccgccgtcgccaagctgatctcgtccaagatgcccagcgacttcaacgccaacgccatccgcgcccacctcgccaacaagtggggcctcggccccctgcgccagaccgccgtcctcctgtacgccatcgcctccgagcctccctcgcgcctggccagctcctcggccgccgaggagtactgggacaacgtcagctcgatgtacgccgagtcgtgcggcatcaccctgcgcccccgccaggacaccatgaacgaggacgccatggcctcgtcggccatcgaccccgccgtcgtcgccgagttctccaagggccaccgcaggctgggcgtccagcagttccaggccctggccgagtacctccagatcgacctgtccggctcccaggccagccagtccgacgccctcgtcgccgagctgcagcagaaggtcgacctgtggaccgccgagatgaccccggagttcctggccggcatctcgccgatgctggacgtcaagaagtcgcgcaggtacggctcctggtggaacatggcccgccaggacgtcctggccttctaccgcaggccctcctacagcgagttcgtcgacgacgccctggccttcaaggtcttcctcaaccgcctgtgcaaccgcgccgacgaggccctcctgaacatggtccgctcgctctcctgcgacgcctacttcaagcagggctccctgccgggctaccacgccgccagccgcctcctggagcaggccatcaccagcaccgtcgccgactgcccgaaggcccgcctcatcctgccggccgtcggcccgcacaccaccatcaccaaggacggcaccatcgagtacgccgaggcccccaggcagggcgtctcgggccccaccgcctacatccagtcgctgcgccagggcgccagcttcatcggcctgaagtcggccgacgtcgacacccagtccaacctcaccgacgccctcctggacgccatgtgcctcgccctgcacaacggcatctccttcgtcggcaagaccttcctggtcaccggcgccggccagggcagcatcggcgccggcgtcgtccgcctcctgctcgagggcggcgcccgcgtcctcgtcaccacctcccgcgagcccgccaccaccagccgctacttccagcagatgtacgacaaccacggcgccaagttctcggagctgcgcgtcgtcccctgcaacctcgcctccgcccaggactgcgagggcctcatccgccacgtctacgacccgcgcggcctcaactgggacctcgacgccatcctgcccttcgccgccgcctccgactactccaccgagatgcacgacatccgcggccagtccgagctgggccaccgcctcatgctggtcaacgtcttccgcgtcctgggccacatcgtccactgcaagcgcgacgccggcgtcgactgccacccgacccaggtcctgctccccctctcgccgaaccacggcatcttcggcggcgacggcatgtacccggagtccaagctcgccctggagagcctcttccaccgcatccgcagcgagtcgtggtccgaccagctgtccatctgcggcgtccgcatcggctggacccgcagcaccggcctcatgaccgcccacgacatcatcgccgagacggtcgaggagcacggcatccgcaccttcagcgtcgccgagatggccctcaacatcgccatgctgctcacccccgacttcgtcgcccactgcgaggacggccccctggacgccgacttcaccggctcgctcggcaccctgggctcgatcccgggcttcctcgcccagctgcaccagaaggtccagctggccgccgaggtcatccgcgccgtccaggccgaggacgagcacgagcgcttcctctccccgggcaccaagcccaccctgcaggcccccgtcgcccccatgcacccccgctcgtccctccgcgtcggctacccccgcctgccggactacgagcaggagatccgccccctcagcccgcgcctggagcgcctgcaggaccccgccaacgccgtcgtcgtcgtcggctactccgagctgggcccctggggctcggcccgcctgcgctgggagatcgagagccagggccagtggacctccgccggctacgtcgagctggcctggctgatgaacctcatccgccacgtcaacgacgagagctacgtcggctgggtcgacacccagaccggcaagccggtccgcgacggcgagatccaggccctctacggcgaccacatcgacaaccacaccggcatccgccccatccagagcacctcgtacaacccggagcgcatggaggtcctccaggaagtcgccgtcgaggaagacctgccggagttcgaggtcagccagctcaccgccgacgccatgcgcctgcgccacggcgccaacgtcagcatccgcccctccggcaaccccgacgcctgccacgtcaagctcaagaggggcgccgtcatcctggtccccaagaccgtcccgttcgtctggggcagctgcgccggcgagctgccgaagggctggacccccgccaagtacggcatccccgagaacctcatccaccaggtcgacccggtcaccctgtacaccatctgctgcgtcgccgaggccttctactccgccggcatcacccaccccctggaggtcttccgccacatccacctgtccgagctcggcaacttcatcggctcgtccatgggcggccccaccaagacccgccagctgtaccgcgacgtctacttcgaccacgagatcccctcggacgtcctccaggacacctacctcaacacccccgccgcctgggtcaacatgctgctcctgggctgcaccggccccatcaagacccccgtcggcgcctgcgccaccggcgtcgagagcatcgactccggctacgagagcatcatggccggcaagaccaagatgtgcctggtcggcggctacgacgacctgcaggaagaggcctcgtacggcttcgcccagctcaaggccaccgtcaacgtcgaggaagagatcgcctgcggccgccagccctcggagatgagccgcccgatggccgagagccgcgccggcttcgtcgaggcccacggctgcggcgtccagctcctgtgccgcggcgacatcgccctgcagatgggcctccccatctacgccgtcatcgcctcctcggccatggccgccgacaagatcggctcctcggtccccgccccgggccagggcatcctctccttcagccgcgagcgcgcccgcagctcgatgatctccgtcacctcccgcccgtcctcgcgctcctccaccagctccgaggtcagcgacaagtccagcctgacctcgatcacctcgatctccaaccccgcccccagggcccagcgcgcccgctcgaccaccgacatggccccgctccgcgccgccctcgccacctggggcctgaccatcgacgacctggacgtcgccagcctgcacggcacctccacccgcggcaacgacctcaacgagcccgaggtcatcgagacgcagatgcgccacctgggccgcaccccgggcaggcccctgtgggccatctgccagaagtccgtcaccggccaccccaaggcccccgccgccgcctggatgctcaacggctgcctgcaggtcctggactcgggcctggtccccggcaaccgcaacctggacaccctggacgaggccctgcgctcggcctcccacctgtgcttccccacccgcaccgtccagctccgcgaggtcaaggccttcctcctgacctccttcggcttcggccagaagggcggccaggtcgtcggcgtcgcccccaagtacttcttcgccaccctgcccaggcccgaggtcgagggctactaccgcaaggtccgcgtccgcaccgaggccggcgaccgcgcctacgccgccgccgtcatgagccaggccgtcgtcaagatccagacccagaacccctacgacgagccggacgccccgcgcatcttcctggaccccctggcccgcatctcccaggaccccagcaccggccagtaccgcttccgctcggacgccacccccgccctcgacgacgacgccctgcccccgcccggcgagccgaccgagctcgtcaagggcatctcgtcggcctggatcgaggagaaggtccgcccccacatgtcccctggcggcaccgtcggcgtcgacctggtccccctggcctccttcgacgcctacaagaacgccatcttcgtcgagcgcaactacaccgtccgcgagcgcgactgggccgagaagtccgccgacgtccgcgccgcctacgcctcccgctggtgcgccaaggaagccgtcttcaagtgcctgcagacgcacagccagggcgccggcgccgccatgaaggagatcgagatcgagcacggtggcaacggcgccccgaaggtcaagctgaggggcgccgcccagaccgccgcccgccagcgcggcctcgagggcgtccagctgtccatcagctacggcgacgacgccgtcatcgccgtcgccctgggcctgatgtcgggcgcctcgtaattaattaaggcaggcaggagttggagtatgagggtagccgctgatggctattcttcccacgtttttgtgtgtttcctcttcatttttttttctcttgccgcaacatgacggctcctgtctctgaagggaacccctgaaattcagggttatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtcaacccaaagtaataagtctgtagtaattggtctcgccctgaattccaaactataaatcaaccactttccctcctcccccccgcccccacttggtcgattcttcgttttctctctaccttctttctattcggttttcttcttcttttattttccctctcccatcaatcaaattcatatttgaaaaaaattaacaatggagtccacacccacgaaacaaaaagctattttttctgcctcgctccttctgttcgccgaacgcgggtttgacgccactacgatgccgatgatcgctgaaaatgctaaggtcggcgcaggaacgatttaccgatactttaagaataaggagagtctggtcaacgagctgttccagcagcacgttaatgaatttttgcaatgtatcgagagtggcttggcgaacgaaagggacggttatcgcgatgggttccatcatatcttcgagggaatggtcacattcacaaagaaccatccgcgcgccttgggatttatcaagacacattcccaaggtacattcctaaccgaagagtcacgccttgcataccaaaaacttgttgagttcgtctgcaccttctttcgagagggacagaaacagggcgtaattcgaaacttgcccgagaatgccctgatcgccatcctattcggatcgtttatggaggtctatgagatgatcgaaaacgattatctctctctaacggatgagttgcttacgggggtagaggaatcgctctgggctgctctctcccgacaatcggctagccctcccaagaagaagcgcaaggtcagcacggccccccccacggacgtctccctcggcgacgagctccacctggacggcgaggacgtcgccatggcccacgccgacgccctcgacgacttcgacctcgacatgctgggcgacggcgacagccccggccccggctttaccccccacgactccgccccctacggcgccctggacatggccgacttcgagtttgagcagatgttcaccgacgccctgggcattgacgagtacggcggctgaggccggccgcgatacccatcatcaacacctgatgttctggggtccctcgtgaggtttctccaggtgggcaccaccatgcgctcacttctacgacgaaacgatcaatgttgctatgcatgagcactcgactatgaatcgaggcacgttaattgagaggctgggaataagggttccatcagaacttctctgggaatgcaaaacaaaagggaacaaaaaaactagatagaagtgaattcatgacttcgacaaccaaatcatcttgtctccgtctgcatacgtgaagcttgtgacgattattctcgcgatgccacgacaaaggttgtgcgaccgtatcttgtccactgtcgtccagtctgcctattccccctccagtgctgccatgtgtcgtaccttgaggtaggtagtctacctaggccagggagctgttagtgcccggctactgggtaatttgtagcgctggagcgattcggtcacaggcgtcaagagtgctgtagcaatgtccgacgccattgatcctgatatcaaataccacctgggcaggtctgggtatgtgaggtcttgtcggatgtgtcgagttcttctccaacgtagtgttcattcgcgctcatggcgcgcctctccttagctctgtacagtgaccggtgactctttctggcatgcggagagacggacggacgcagagagaagggctgagtaataagcgccactgcgccagacagctctggcggctctgaggtgcagtggatgattattaatcagggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtctgtttaaacggatccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgtgacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 54 - DNA, 20 bp, Artificial Sequence
  Synthetic Polynucleotide
tgcgacaagagcatgatccg

SEQ ID NO: 55 - DNA, 21 bp, Artificial Sequence
  Synthetic Polynucleotide
ggcacttctcgatgttgttcg

SEQ ID NO: 56 - DNA, 49 bp, Artificial Sequence
  Synthetic Polynucleotide
catactccaactcctgcctgccttaattaattacttgcgcggggtgtag

SEQ ID NO: 57 - DNA, 33 bp, Artificial Sequence
  Synthetic Polynucleotide
ctagtccctcacaccatggccgtcaagcacctc

SEQ ID NO: 58 - DNA, 24 bp, Artificial Sequence
  Synthetic Polynucleotide
atcacctcggaggtcgccgagacg

SEQ ID NO: 59 - DNA, 24 bp, Artificial Sequence
  Synthetic Polynucleotide
atcacctcggaggtcgccgagacg

SEQ ID NO: 60 - DNA, 24 bp, Artificial Sequence
  Synthetic Polynucleotide
acaacctctcgatggtcagcttcc

SEQ ID NO: 61 - DNA, 24 bp, Artificial Sequence
  Synthetic Polynucleotide
acaacctctcgatggtcagcttcc

SEQ ID NO: 62 - DNA, 24 bp, Artificial Sequence
  Synthetic Polynucleotide
tggtcaagctcgtcaacaagtggc

SEQ ID NO: 63 - DNA, 24 bp, Artificial Sequence
  Synthetic Polynucleotide
gttgcggatccagttcaggtgctt

SEQ ID NO: 64 - DNA, 24 bp, Artificial Sequence
  Synthetic Polynucleotide
gctggaagcagtacccgttcacca

SEQ ID NO: 65 - DNA, 24 bp, Artificial Sequence
  Synthetic Polynucleotide
tcgcgggtctggaagatgaggcag

SEQ ID NO: 66 - DNA, 20 bp, Artificial Sequence
  Synthetic Polynucleotide
tgcaccttctcgttccagac

SEQ ID NO: 67 - DNA, 20 bp, Artificial Sequence
  Synthetic Polynucleotide
gatgatcaggccgaagaggg

SEQ ID NO: 68 - DNA, 20 bp, Artificial Sequence
  Synthetic Polynucleotide
ccgagctcgacttctccatc

SEQ ID NO: 69 - DNA, 20 bp, Artificial Sequence
  Synthetic Polynucleotide
tagtcctcgagcgagtcgaa

SEQ ID NO: 70 - DNA, 20 bp, Artificial Sequence
  Synthetic Polynucleotide
acctacgccatcctgtccaa

SEQ ID NO: 71 - DNA, 20 bp, Artificial Sequence
  Synthetic Polynucleotide
aagctgtgcttcttgagcga

SEQ ID NO: 72 - DNA, 20 bp, Artificial Sequence
  Synthetic Polynucleotide
acctcgtaccacatccccat

SEQ ID NO: 73 - DNA, 20 bp, Artificial Sequence
  Synthetic Polynucleotide
gagacgtccgacttgaggac

SEQ ID NO: 74 - DNA, 22 bp, Artificial Sequence
  Synthetic Polynucleotide
ccttcaaggtcttcctcaaccg

SEQ ID NO: 75 - DNA, 24 bp, Artificial Sequence
  Synthetic Polynucleotide
gttgtcgtacatctgctggaagta

SEQ ID NO: 76 - DNA, 24 bp, Artificial Sequence
  Synthetic Polynucleotide
agttgatgttgtagttgacgacct

SEQ ID NO: 77 - DNA, 24 bp, Artificial Sequence
  Synthetic Polynucleotide
gacctcctacaccttcagctactc

SEQ ID NO: 78 - DNA, 24 bp, Artificial Sequence
  Synthetic Polynucleotide
aacccttcccgacaaccgctccac

SEQ ID NO: 79 - DNA, 24 bp, Artificial Sequence
  Synthetic Polynucleotide
gctgtctcggatctggaccaagtg

SEQ ID NO: 80 - DNA, 24 bp, Artificial Sequence
  Synthetic Polynucleotide
ttaccttacaagagctcgatctgc

SEQ ID NO: 81 - DNA, 24 bp, Artificial Sequence
  Synthetic Polynucleotide
aagtcacgctcgacgtacagatcg

SEQ ID NO: 82 - DNA, 20 bp, Artificial Sequence
  Synthetic Polynucleotide
aacctcgagacgctcttcta

SEQ ID NO: 83 - DNA, 18 bp, Artificial Sequence
  Synthetic Polynucleotide
atccacttgcttcacgct

SEQ ID NO: 84 - DNA, 18 bp, Artificial Sequence
  Synthetic Polynucleotide
gacgcccagcatttcatc

SEQ ID NO: 85 - DNA, 20 bp, Artificial Sequence
  Synthetic Polynucleotide
agcgtgacccactcaggtaa

SEQ ID NO: 86 - DNA, 11806 bp, Artificial Sequence
  Synthetic Polynucleotide
gtttaaaccccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtatttaaatctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctggcgcgccgacgtgtatcataagttaagtaggtactgtacgttagtttgtttaccttcttcagccagtagtccgactttgctctcaagtgctcatccaaccctttggccttccaaatcttgataccgagagcctcaggcaggttggtatattctacctcaaatacctgccagatgttgaccaatgccgttgtcgttactccgtactacgcgaaccaatgccaacatgattctcctttcagtcacgagacgattcgagtgtcatggtagcagtactttggtagtaaagagtcactcaattgaacatgtcgtagctcattcgtaaccaagtcatgataaccctgaatttcaggggttcccttcagagacaggagccgtcatgttgcggcaagagaaaaaaaaatgaagaggaaacacacaaaaacgtgggaagaatagccatcagcggctaccctcatactccaactcctgcctgccttaattaattagtggcggtggcgcgggaggggcgggatgctctgctcgttgcggaagaagttgttggggtcgacgagggtcttgaccttgaccaggcggtcgaagttcttgccgaagtacttctcgccccagatgcgggcctgggtgtagttgttcgggttcttggggtcgttgatgccgatgtcgaggtcgcggtagttgaggtaggccaggcgcgggttcttgctgacgtagggggtcatgaagttgtagatgttgcggatccagttcaggtgcttctcgttgtcttcctgcttctcccaggagcagatgtaccagagctcgtacaggatgccggcgcggtgggggaaggggatggccgactcggagatctcgtccatgatgccaccgtacgggtagagggcgtacatgccggcgccgatgtcttcctcgtagagcttctccaggatctggacgaagacggactccgggatgggcttcttgacgtagtccagcttgatcttgaaggcgccgttctggccggcggagcggtccagcaggatctccttgttgaagttgtcggtgtcgtagttgacgacgcccgagtagaagatgatggtgtcgatccagctgagctggcggcagtcggtcttcttgatgcccagctccgggaaggacttgttcatgaggtcgaccagggagtcgacgccgccgaggaagacggacgagaagtaggtgtggatggcggtcttgttcttgccctggttgtcggtgatgttgcgggtgatgaagtgggtcatgagcaggaggtccttgtcgtacttgtaggcgatgttctgccacttgttgacgagcttgaccagctcgtggatctccatgatcttcttgacggagaacatggtcgacttggggacggcgaccaggcggatcttccaggcgacgatgatgccgaagctctcggcgccgccgcccctgagggcccagaacaggtcctcgcccatggacttgcggtcgaggaccttgccgtggacgttgaccaggtgggcgtcgatgatgttgtcggcggcgaggccgtagttgcgcatcagggggccgtagccgccgccaccgaagtggccgccagcgcagacggtggggcagtagccggcggccagggacaggttctcgttcttctcgttgacccagtagtagacctcgccgagggtggcgccggcctcgacccaggcggtctggctgtggacgtcgatcttgatggagcgcatgttgcgcaggtcgacgatgacgaaggggacctgcgagatgtagctcatgccctccgagtcgtggccgccgctgcgggtgcggatctggaggccgaccttcttcgagcacaggatggtgccctggatgtgggagacgtgcgacggggtgacgatgacgagcggcttgggggtggtgtcgctggtgaagcgcaggttgtggatggtgctgttgaggacggacatgtacagggggttgttctgggtgtagacgagcttcaggttggtggcgttgttcgggatgtactgcgagaagcacttgaggaagttctcgcgggggttcatggtgtgagggactaggtaggcttttgtagtgttgcagggagttggaggggatgtgcgacttctggtttgcttctcttaggttgagtatgaaagttgaggacgactcggggtcaagacgtcctgagagagagcgggagcatgctggcccccgacgccttcttaagcaaaccagctcatggcgcgtcagactggattcggaagatcgaccgggaacgagtaagggccagtggttggcacctctcgggcagcagcagcagcagcagcagcagcaatgtgccatggcatctgcgcgatcgggcatcgttgaccgctgttcccgcaggcgatgtaccatgttatccgcgcctgcctgcttgcgagtggtgccatggcaaatgctggaagcgggtccctcgctacagagtaaatccacggctgcaggagacgcgcagttggtcatccctggggcccctgcgccacgcggcactgccttacccctctgcacacgcgtgactaacccccactactgagtaccccgcttgtcaaaaggtcgcttccatacttatcgccagcctgacattatcgcgtctgcactggaaacctaagcgggtaaagcatcagagcatcaaatccaaggctctttctcctatctctgtaaatgagaggacaagttgatttcggaatcccgagtagaacggcagacagccaggcatactatcattacgcagctccggggaaagatccgacaaccagagccagtctctttctgccgttctgatgattccatcttcccctcagctccttcaccgcccagcgtctgctacgtgtccggcccggctttgcctgcctcgtcctgcagccaacgggactgcgcgaccgagccgccgactctgcaagtaatggtacctaacgaccgccccaagctggtagctctgtcctggtttcgccgcgtaagtctcggcgctagccttgattatgctgtctcggatctggaccaagtgtttcgatattccatcccatgacctacatgcgccggcgaatgccctccgtccccttccttcacaacctcgaattcctcccaatgtggatatctgtcgcctttctaagaaagggcgtggaacacgcgcgattagtatagaatatggatcgacctaagttgtctccgcacatgtctcaacagtctagcgacaagaagaacctcgcccacccgtcgattacgagcgtgtgcagcctgagtgtgtgtgagttggagttaacggcgccgaaatctgaaggagggaagagacttttcaacacgtctgttctctactgactttttttgtttttaccacatcgcactaggaaagctagcggtgttttgatggtgccaatactgcgtaactgcgtaatgttgcatattgcgtagcagcgtgaagcaagtggatgtatgtacggactaatccgtatgcactgcatctcgcagcagatggcacctccccagagacagccgggaaacaagcttttttttccttggcgtccttggcttgcatggcttcattggcgggtgttatgttttccccaggggtgcagcaatggcacgccgagcaaaaaaggaaccgcggacctggcacaagccccaaactccatcgacgcaggacggcatcgcattgcgttgcgcctcctctccaacgacgtcttaagggaaaagaaaagaaaaacaaaaggataatggcaggcctccagcaagcaagcaagagcgcttccggccgttcaagggtccaatccggttcaagccttgcgttatcgtcccagagggcggcccttgttgtaagccggccttgtgttcgcgcccttcgatgtttgacattgcttttccgtctgggtactttccagtcggttgttggagacttcctcgtcattgtatggggtcacatgttctctgcgcactacactacggagtaagtgctaataaattacatctcgaccccgtgcttggcaacaacctcgaggaacctgtcctgcttgccttatttccgtcggttgggtagacggcttgttcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 87 - DNA, 11804 bp, Artificial Sequence
  Synthetic Polynucleotide
gtttaaaccccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtatttaaatctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctggcgcgccatgtaggtcatgggatggaatatcgaaacacttggtccagatccgagacagcataatcaaggctagcgccgagacttacgcggcgaaaccaggacagagctaccagcttggggcggtcgttaggtaccattacttgcagagtcggcggctcggtcgcgcagtcccgttggctgcaggacgaggcaggcaaagccgggccggacacgtagcagacgctgggcggtgaaggagctgaggggaagatggaatcatcagaacggcagaaagagactggctctggttgtcggatctttccccggagctgcgtaatgatagtatgcctggctgtctgccgttctactcgggattccgaaatcaacttgtcctctcatttacagagataggagaaagagccttggatttgatgctctgatgctttacccgcttaggtttccagtgcagacgcgataatgtcaggctggcgataagtatggaagcgaccttttgacaagcggggtactcagtagtgggggttagtcacgcgtgtgcagaggggtaaggcagtgccgcgtggcgcaggggccccagggatgaccaactgcgcgtctcctgcagccgtggatttactctgtagcgagggacccgcttccagcatttgccatggcaccactcgcaagcaggcaggcgcggataacatggtacatcgcctgcgggaacagcggtcaacgatgcccgatcgcgcagatgccatggcacattgctgctgctgctgctgctgctgctgcccgagaggtgccaaccactggcccttactcgttcccggtcgatcttccgaatccagtctgacgcgccatgagctggtttgcttaagaaggcgtcgggggccagcatgctcccgctctctctcaggacgtcttgaccccgagtcgtcctcaactttcatactcaacctaagagaagcaaaccagaagtcgcacatcccctccaactccctgcaacactacaaaagcctacctagtccctcacaccatgaacccccgcgagaacttcctcaagtgcttctcgcagtacatcccgaacaacgccaccaacctgaagctcgtctacacccagaacaaccccctgtacatgtccgtcctcaacagcaccatccacaacctgcgcttcaccagcgacaccacccccaagccgctcgtcatcgtcaccccgtcgcacgtctcccacatccagggcaccatcctgtgctcgaagaaggtcggcctccagatccgcacccgcagcggcggccacgactcggagggcatgagctacatctcgcaggtccccttcgtcatcgtcgacctgcgcaacatgcgctccatcaagatcgacgtccacagccagaccgcctgggtcgaggccggcgccaccctcggcgaggtctactactgggtcaacgagaagaacgagaacctgtccctggccgccggctactgccccaccgtctgcgctggcggccacttcggtggcggcggctacggccccctgatgcgcaactacggcctcgccgccgacaacatcatcgacgcccacctggtcaacgtccacggcaaggtcctcgaccgcaagtccatgggcgaggacctgttctgggccctcaggggcggcggcgccgagagcttcggcatcatcgtcgcctggaagatccgcctggtcgccgtccccaagtcgaccatgttctccgtcaagaagatcatggagatccacgagctggtcaagctcgtcaacaagtggcagaacatcgcctacaagtacgacaaggacctcctgctcatgacccacttcatcacccgcaacatcaccgacaaccagggcaagaacaagaccgccatccacacctacttctcgtccgtcttcctcggcggcgtcgactccctggtcgacctcatgaacaagtccttcccggagctgggcatcaagaagaccgactgccgccagctcagctggatcgacaccatcatcttctactcgggcgtcgtcaactacgacaccgacaacttcaacaaggagatcctgctggaccgctccgccggccagaacggcgccttcaagatcaagctggactacgtcaagaagcccatcccggagtccgtcttcgtccagatcctggagaagctctacgaggaagacatcggcgccggcatgtacgccctctacccgtacggtggcatcatggacgagatctccgagtcggccatccccttcccccaccgcgccggcatcctgtacgagctctggtacatctgctcctgggagaagcaggaagacaacgagaagcacctgaactggatccgcaacatctacaacttcatgaccccctacgtcagcaagaacccgcgcctggcctacctcaactaccgcgacctcgacatcggcatcaacgaccccaagaacccgaacaactacacccaggcccgcatctggggcgagaagtacttcggcaagaacttcgaccgcctggtcaaggtcaagaccctcgtcgaccccaacaacttcttccgcaacgagcagagcatcccgcccctcccgcgccaccgccactaattaattaaggcaggcaggagttggagtatgagggtagccgctgatggctattcttcccacgtttttgtgtgtttcctcttcatttttttttctcttgccgcaacatgacggctcctgtctctgaagggaacccctgaaattcagggttatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtcgccggcgaatgccctccgtccccttccttcacaacctcgaattcctcccaatgtggatatctgtcgcctttctaagaaagggcgtggaacacgcgcgattagtatagaatatggatcgacctaagttgtctccgcacatgtctcaacagtctagcgacaagaagaacctcgcccacccgtcgattacgagcgtgtgcagcctgagtgtgtgtgagttggagttaacggcgccgaaatctgaaggagggaagagacttttcaacacgtctgttctctactgactttttttgtttttaccacatcgcactaggaaagctagcggtgttttgatggtgccaatactgcgtaactgcgtaatgttgcatattgcgtagcagcgtgaagcaagtggatgtatgtacggactaatccgtatgcactgcatctcgcagcagatggcacctccccagagacagccgggaaacaagcttttttttccttggcgtccttggcttgcatggcttcattggcgggtgttatgttttccccaggggtgcagcaatggcacgccgagcaaaaaaggaaccgcggacctggcacaagccccaaactccatcgacgcaggacggcatcgcattgcgttgcgcctcctctccaacgacgtcttaagggaaaagaaaagaaaaacaaaaggataatggcaggcctccagcaagcaagcaagagcgcttccggccgttcaagggtccaatccggttcaagccttgcgttatcgtcccagagggcggcccttgttgtaagccggccttgtgttcgcgcccttcgatgtttgacattgcttttccgtctgggtactttccagtcggttgttggagacttcctcgtcattgtatggggtcacatgttctctgcgcactacactacggagtaagtgctaataaattacatctcgaccccgtgcttggcaacaacctcgaggaacctgtcctgcttgccttatttccgtcggttgggtagacggcttgttcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacg

SEQ ID NO: 88 - DNA, 972 bp, Artificial Sequence
  Synthetic Polynucleotide
atgtcggccggctcggaccagatcgagggctcgccccaccacgagagcgacaactcgatcgccaccaagatcctgaacttcggccacacctgctggaagctccagcgcccgtacgtcgtcaagggcatgatctccatcgcctgcggcctgttcggccgcgagctcttcaacaaccgccacctgttctcgtggggcctcatgtggaaggccttcttcgccctggtcccgatcctctccttcaacttcttcgccgccatcatgaaccagatctacgacgtcgacatcgaccgcatcaacaagccggacctgccgctcgtctcgggcgagatgtccatcgagacggcctggatcctcagcatcatcgtcgccctgaccggcctcatcgtcaccatcaagctgaagtcggccccgctcttcgtcttcatctacatcttcggcatcttcgccggcttcgcctacagcgtcccgcccatccgctggaagcagtacccgttcaccaacttcctgatcaccatctcgtcccacgtcggcctcgccttcacctcctactcggccaccaccagcgccctgggcctccccttcgtctggcgcccggccttctcgttcatcatcgccttcatgaccgtcatgggcatgaccatcgccttcgccaaggacatctcggacatcgagggcgacgccaagtacggcgtctccaccgtcgccaccaagctgggcgcccgcaacatgaccttcgtcgtcagcggcgtcctcctgctcaactacctcgtctcgatctccatcggcatcatctggccccaggtcttcaagtccaacatcatgatcctcagccacgccatcctggccttctgcctcatcttccagacccgcgagctggccctcgccaactacgcctccgccccgagccgccagttcttcgagttcatctggctcctctactacgccgagtacttcgtctacgtcttcatctga

SEQ ID NO: 89 - protein, 323 aa, Cannabis sativa
MSAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 90 - protein, 517 aa, Cannabis sativa
MNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 91 - DNA, 1554 bp, Artificial Sequence
  Synthetic Polynucleotide
atgaacccccgcgagaacttcctcaagtgcttctcgcagtacatcccgaacaacgccaccaacctgaagctcgtctacacccagaacaaccccctgtacatgtccgtcctcaacagcaccatccacaacctgcgcttcaccagcgacaccacccccaagccgctcgtcatcgtcaccccgtcgcacgtctcccacatccagggcaccatcctgtgctcgaagaaggtcggcctccagatccgcacccgcagcggcggccacgactcggagggcatgagctacatctcgcaggtccccttcgtcatcgtcgacctgcgcaacatgcgctccatcaagatcgacgtccacagccagaccgcctgggtcgaggccggcgccaccctcggcgaggtctactactgggtcaacgagaagaacgagaacctgtccctggccgccggctactgccccaccgtctgcgctggcggccacttcggtggcggcggctacggccccctgatgcgcaactacggcctcgccgccgacaacatcatcgacgcccacctggtcaacgtccacggcaaggtcctcgaccgcaagtccatgggcgaggacctgttctgggccctcaggggcggcggcgccgagagcttcggcatcatcgtcgcctggaagatccgcctggtcgccgtccccaagtcgaccatgttctccgtcaagaagatcatggagatccacgagctggtcaagctcgtcaacaagtggcagaacatcgcctacaagtacgacaaggacctcctgctcatgacccacttcatcacccgcaacatcaccgacaaccagggcaagaacaagaccgccatccacacctacttctcgtccgtcttcctcggcggcgtcgactccctggtcgacctcatgaacaagtccttcccggagctgggcatcaagaagaccgactgccgccagctcagctggatcgacaccatcatcttctactcgggcgtcgtcaactacgacaccgacaacttcaacaaggagatcctgctggaccgctccgccggccagaacggcgccttcaagatcaagctggactacgtcaagaagcccatcccggagtccgtcttcgtccagatcctggagaagctctacgaggaagacatcggcgccggcatgtacgccctctacccgtacggtggcatcatggacgagatctccgagtcggccatccccttcccccaccgcgccggcatcctgtacgagctctggtacatctgctcctgggagaagcaggaagacaacgagaagcacctgaactggatccgcaacatctacaacttcatgaccccctacgtcagcaagaacccgcgcctggcctacctcaactaccgcgacctcgacatcggcatcaacgaccccaagaacccgaacaactacacccaggcccgcatctggggcgagaagtacttcggcaagaacttcgaccgcctggtcaaggtcaagaccctcgtcgaccccaacaacttcttccgcaacgagcagagcatcccgcccctcccgcgccaccgccactaa